(19) United States
(12) Patent Application Publication
(10) Pub. No.: US 2002/0127605 A1
(43) Pub. Date: Sep. 12, 2002

(54) Bisubstituted carbocyclic cyclophilin binding compounds and their use

(76) Inventors: Gregory S. Hamilton, Catonsville, MD (US); Sergei Belyakov, Baltimore, MD (US); Mark Vaal, Baltimore, MD (US); Ling Wei, Lutherville, MD (US); Yong-Qian Wu, Columbia, MD (US); Joseph P. Steiner, Mt. Airy, MD (US)

Correspondence Address: Michael J. Bell, HOWREY SIMON ARNOLD & WHITE, LLP, Box No. 34, 1299 Pennsylvania Avenue, N.W., Washington, DC, 20004-2402, US

(21) Appl. No.: 09/994,927

(22) Filed: Nov. 28, 2001

Related U.S. Application Data
(60) Provisional application No. 60/253,074, filed on Nov. 28, 2000.
(60) Provisional application No. 60/291,966, filed on May 21, 2001.

(51) Int. Cl. G01N 33/53; C07C 311/50; C07C 311/15; C07C 275/28; C07C 281/06

(52) U.S. Cl. 435/7.1; 564/18; 564/34; 564/48; 564/39; 564/148; 564/82; 564/84

(57) ABSTRACT

The present invention relates to novel, non-peptidic small organic compounds having an affinity for cyclophilin (CyP)-type immunophilin proteins. In the compounds of this invention, at least two carbo- or heterocyclic groups are attached to a central saturated, partially saturated, or aromatic 5-6 membered carbocyclic ring by a combination of straight or branched linker chains. The invention further relates to pharmaceutical compositions comprising one or more of the said compounds, and to the uses of these compounds and compositions for binding CyP-type proteins, inhibiting their peptidyl-prolyl isomerase activity, and for research, development, and therapeutic applications in a variety of medical disorders, such as neurological disorders, hair loss disorders, ischemic disorders, and disorders caused by viral or protozoan infection.

A claim is hereby made for the benefit under 35 U.S.C 119(e) of U.S. provisional application Ser. No. 60/253,074 filed Nov. 28, 2000, and of U.S. provisional application Ser. No. 60/291,966 filed May 21, 2001, both of which are incorporated herein by reference in their entirety.

The present invention relates to novel, non-peptidic small organic compounds having an affinity for cyclophilin (CyP)-type immunophilin proteins. The invention further relates to the uses of these compounds for binding CyP-type proteins, inhibiting their peptidyl-prolyl isomerase activity, and for research, development, and therapeutic applications in a variety of medical conditions.

Immunophilins are a group of proteins which are functionally characterized by their ability to bind certain immunosuppressive drugs. Two structurally and pharmacologically distinct classes of immunophilins are the FK506 binding proteins (FKBPs) and the cyclophilin (CyP) proteins. Although the prototypical members of these two protein families, FKBP12 and cyclophilin A, are both involved in the cellular mechanisms which mediate immunosuppression, they display selective affinities for very different types of immunosuppressants: members of the FKBP family bind to the macrolide antibiotics FK506 and rapamycin, whereas members of the CyP family bind to the cyclic undecapeptide Cyclosporin A (CsA).

Common to all immunosuppressant drugs is their ability to interfere with the intracellular signalling cascades of cells of the immune system. In the case of FK506 and CsA, binding of these drugs to their respective receptor proteins FKBP12 and cyclophilin A results in the cross-linking of the intracellular phosphatase calcineurin to the drug-receptor complex. The resulting inactivation of calcineurin eventually leads to the accumulation of phosphorylated calcineurin substrates, including the signaling protein NFAT (nuclear factor of activated T-cells). NFAT plays an important role in the regulation and transcriptional activation of genes involved in the T-cell activation prong of the immune response. Absent calcineurin activity, the T-cell activation cascade is interrupted because NFAT, in its phosphorylated state, cannot translocate to the cell nucleus.

Apart from its effects on the immune system, CsA has been shown to possess biological activity in the central nervous system. In rodent models of cerebral stroke, systemic treatment with CsA either before or following occlusion of the medial cerebral artery causes a reduction of infarct size [T. Yoshimoto and B. K. Siesjo, Brain Res., 839, pp. 283-91 (1999)]. CsA also protects against the decrease of acetyl choline receptors observed in the hippocampal formation after transient global forebrain ischemia [Y. Kondo et al., Neurochem Res., 24, pp. 9-13 (1999)], and has demonstrable neuroprotective effects in animal models of insulin-induced hypoglycemic coma [H. Friberg et al., J Neurosci., 18, pp. 5151-9 (1998)], traumatic brain injury [P. G. Sullivan et al., Exp Neurol., February 2000; 161, 631-7 (2000)], and experimental dopamine neuron degeneration [K. Matsuura et al., Exp. Neurol., 146, 526-351 (1997)]. In order for CsA to exert a protective effect after neural insult, it must be available at the site of injury. However, due to the blood-brain barrier, CsA shows only very limited penetration into the brain when administered systemically, and its best beneficial effects are seen if the blood-brain barrier is compromised [H. Uchino et al., Brain Res., 812, pp. 216-26 (1998); P. G. Sullivan et al., Exp. Neurol., 161, pp. 631-7 (2000)].

While the present invention is not bound by any particular theory, it appears that at least some of the effects of CsA on cells of the nervous system occur independently of calcineurin inhibition. Some of the inventors have previously shown that non-immunosuppressive peptidic analogues of CsA, which lack a calcineurin-binding domain, display neurotrophic activity in neural cell culture which is equal to that of CsA [J. P. Steiner et al., Nat. Med., 3, pp. 421-8 (1997)].

A number of types of mammalian cyclophilins have been identified and cloned, cyclophilins A, B, C, D, and cyclophilin-40 [Snyder and Sabatini, Nat. Med. 1:32-37 (1995); Friedman et al., Proc. Natl. Acad. Sci., 90:6815-6819 (1993)]. Cyclophilin B possesses an N-terminal signal sequence that directs translocation into the endoplasmic reticulum of the cell. The 23 kD cyclophilin C is found in the cytosol of the cell. Cyclophilin D, at 18 kD, appears to target its actions in the mitochondria, and cyclophilin-40 is a component of the inactivated form of a glucocorticoid receptor. Cyclophilin A is a 19 kD protein, which is abundantly expressed in a wide variety of cells. Like the other cyclophilins, cyclophilin A not only binds the immunosuppressive agent CsA, but it also possesses peptidyl-prolyl cis-trans isomerase (PPIase) and protein folding or “chaperone” activities. PPIase activity catalyzes the conversion of proline residues in a protein from the cis to the trans conformation [Fischer, et al., Biomed. Biochem. Acta 43:1101-1112 (1984)].

Since cyclophilin A was first identified as the receptor for CsA, the effects of the CsA:cyclophilin interaction have been well documented. Cyclosporin A binds to cyclophilin A with a dissociation constant in the range of 10⁻⁸ mol/L, a value representing a relatively high degree of affinity [Handschumacher et al., Science 226:544 (1984)]. Knowledge about the interaction between drug and protein spawned a number of drug discovery efforts. Initially, the focus was on identifying novel immunosuppressive drugs that would mimic the effects of CsA without displaying its dose-limiting side-effects.

The field, however, lacks appreciation of the usefulness of cyclophilin-binding compounds for treating disease states, injuries and other abnormal conditions involving the central nervous system and other parts of the body. For therapeutic application in disorders of the central nervous system, for example, cyclophilin-binding compounds would need to penetrate from the bloodstream into the brain to bind to cyclophilin and exert biological effects. Cyclosporin A, however, generally displays poor penetration into the central nervous system after systemic administration, and therefore possess only low therapeutic potential for CNS applications if the blood-brain barrier is intact. See Uchino et al.; Sullivan et al., supra. Therefore, there exists need for safe and effective compositions and methodologies for treating disease states, injuries and other abnormal conditions involving the central nervous system and other organs by use of cyclophilin-binding compounds. These needs have gone unresolved until the development of the present inventions. Researchers have also noted a functional association of cyclophilin A with the Gag protein of the HIV virus [Thali et al., Nature 372:363-365(1994)]. This has taken drug development approaches in a new direction (See, for example, U.S. Pat. No. 5,767,069). Many researchers now seek to develop drugs that target the interaction between cyclophilin A and Gag in order to disrupt the HIV life cycle [Stemberg, BioWorld Today 7:1 (1996)].

### SUMMARY OF THE INVENTION

The focus of the present invention is on non-peptidic small molecule compounds which interact with, have an affinity for, or bind to cyclophilin proteins. By studying the binding interaction of cyclophilin A and CsA, the inventors have designed and characterized a number of novel small molecule organic compounds which interact with cyclophilins, on the basis of which the inventors were able to develop and utilize screening procedures for rapidly identifying a class of similarly active compounds. These compounds have been specifically tested to show that they effect the growth and regeneration of cells of the nervous system, and protect such cells from otherwise lethal chemical injury. The compounds can be used in a number of ways, including therapeutic and research and development applications for various medical conditions, including neurological disorders.

The invention thus provides compounds that bind to CyP proteins. The compounds of this invention preferably do not suppress the immune system and preferably do not possess a biological activity involving binding to a FKBP, i.e., the compounds inhibit the peptidyl prolyl isomerase activity of FKBP with an IC₅₀ of greater than 500 nM. A number of methods for determining the binding to CyPs and ways for exploiting the binding through in vitro and in vivo methods and uses are presented. Preferred compounds function to promote or affect neuronal cell growth or growth of nervous system cells, regenerate damaged or diseased neurons, or protect neurons or neuronal cells from death or degeneration following damage. Furthermore, aspects of this invention can be used in methods to identify and isolate additional CyP binding compounds or additional uses of the compounds.

The invention also provides a number of uses for these compounds, including uses that comprise the step of allowing the compound to contact an immunophilin protein. A variety of permutations of this method can be devised. In particular, the compounds can be used to affect the growth or resistance to noxious stimuli of neuronal cells, either in culture or in an animal. Thus, the compounds can be administered to cells or animals to affect a number of conditions associated with the decline, damage, or degeneration of nervous system cells or their physiological function.

In one aspect, the invention provides compounds of Formula I as shown and described below:

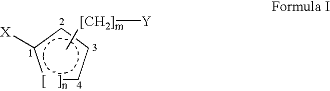

where n is 1 or 2, forming a central 5-6 membered carbocyclic ring which is optionally saturated, partially saturated, or aromatic;

m is 0-3;

—(CH₂)ₘ—Y is attached to said central carbocyclic ring at position 2, 3, or 4;

X and Y are the same or different, and may independently be:

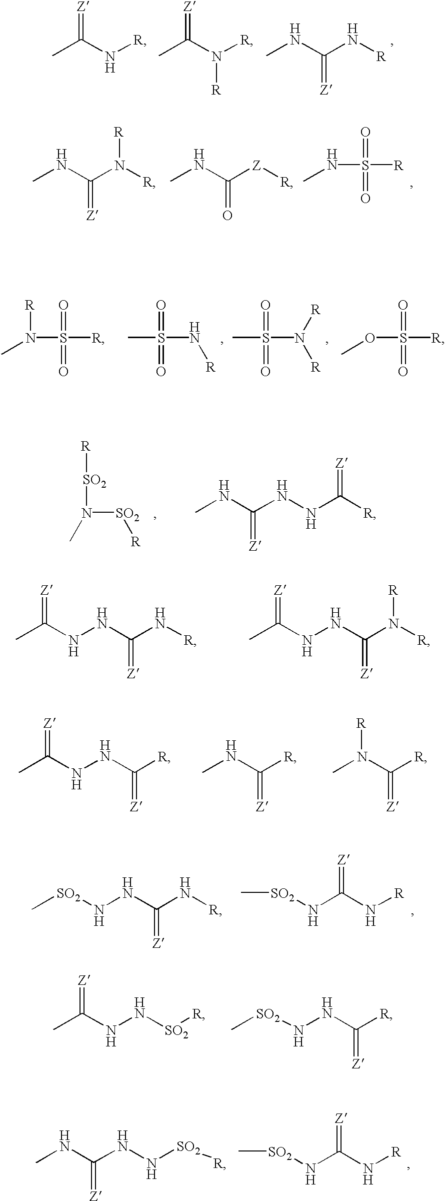

or a combination thereof,

or C₁-C₆ straight or branched chain alkyl, alkenyl, or alkynyl which is substituted at one or several positions with Q, and which further may optionally be substituted at one or several positions by hydroxyl, mercaptyl, or carbonyl oxygen;

and where Y may further be: Q,

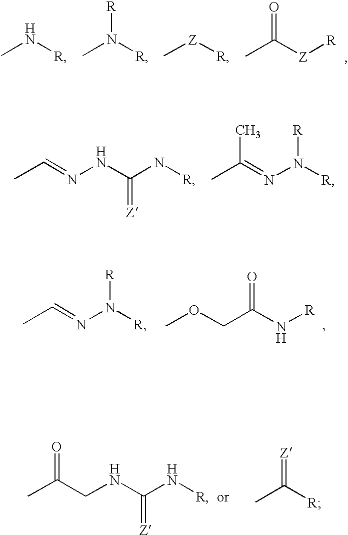

wherein Z′ is O, S, N(CN), CH(NO₂), or N(NO₂);

Z is O or S; and

R may independently be:

Q,

or C₁-C₆ straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;

and wherein Q, which is optionally saturated, partially saturated, or aromatic, is a mono-, bi-, or tricyclic, carbo- or heterocyclic ring, which is optionally and independently substituted in one or several positions with a substituent selected from the group consisting of halo; hydroxyl; mercaptyl; nitro; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl; and wherein the individual ring sizes are 5-6 members, and wherein each heterocyclic ring contains 1-6 heteroatoms independently selected from the group consisting of O, N, and S in any chemically stable order and oxidation state;

provided that:

when R is Q, or Q-substituted C₁-C₆ alkyl or alkenyl, or Q-substituted C₁-C₆ alkyl or alkenyl which is additionally substituted with one or more hydroxyl- or oxo-groups, and

n is 2, and

m is 0, and

Y is attached to said central carbocyclic ring at position 3;

then X and Y are not both

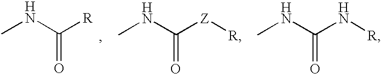

or a combination thereof;

and further provided that:

when R is Q, or Q-substituted C₁-C₆ alkyl or alkenyl, or Q-substituted C₁-C₆ alkyl or alkenyl which is additionally substituted with one or more hydroxyl- or oxo-groups, and

n is 2, and

m is 0, and

Y is attached to said central carbocyclic ring at position 3,

and said carbocyclic ring is aromatic;

then X and Y are not both:

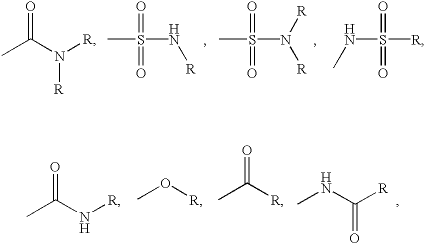

or a combination thereof.

Preferred embodiments of the present invention are exemplified by the following compounds of formulae II-VII. Compounds of formula II have the following general structure:

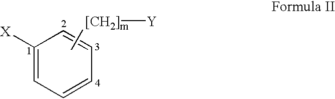

wherein m, X, Y, R, Z, and Z′ are as defined in formula I, the substituent —[CH₂]ₘ—Y is attached at position 2, or 3; and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; mercaptyl; nitro; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state;

provided that:

when R is Q, or Q-substituted C₁-C₆ alkyl or alkenyl, or Q-substituted C₁-C₆ alkyl or alkenyl which is additionally substituted with one or more hydroxyl- or oxo-groups, and

m is 0, and

Y is attached at position 3;

then X and Y are not both

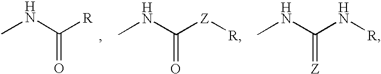

or a combination thereof;

and further provided that:

when R is Q, or Q-substituted C₁-C₆ alkyl or alkenyl, or Q-substituted C₁-C₆ alkyl or alkenyl which is additionally substituted with one or more hydroxyl- or oxo-groups, and

m is 0, and

Y is attached at position 3,

then X and Y are not both:

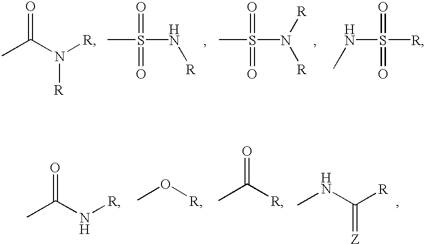

or a combination thereof.

Compounds of formula IIa have the following general structure:

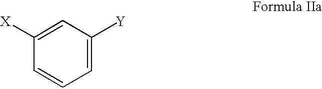

where X and Y are the same or different, and may independently be:

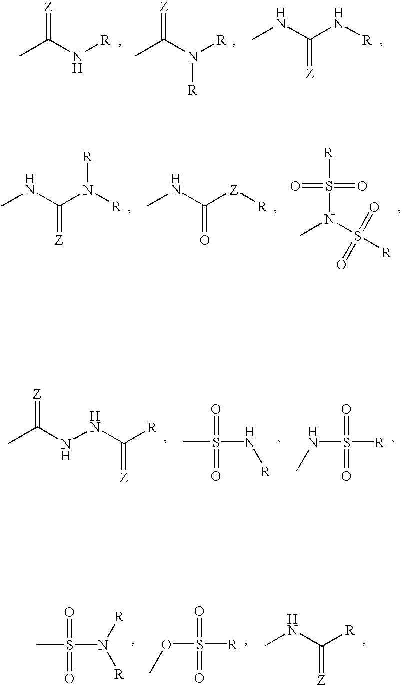

or a combination thereof, or C1-C6 straight or branched chain lower alkyl, alkenyl, or alkynyl which is substituted at one or several positions with Q, and which further may optionally be substituted at one or several positions by hydroxyl, mercaptyl, or carbonyl oxygen;

and where Y may further be: Q,

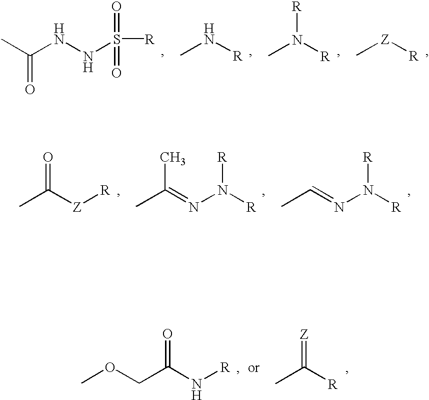

wherein Z is O or S, and

R may independently be:

Q,

or C1-C6 straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;

and wherein Q is a mono-, bi- or tricyclic carbo- or heterocyclic ring which is optionally saturated, partially saturated, or aromatic, and which may optionally be substituted in one or several positions with halo, hydroxyl, mercaptyl, nitro, cyano, trifluoromethyl, C1-C6 straight or branched chain alkyl or -alkenyl, C1-C4 alkoxy or -alkenyloxy, phenoxy, benzyloxy, amino, or acetyl, and wherein the individual ring sizes are 5-6 members, and wherein each heterocyclic ring contains 1-6 heteroatoms selected from the group consisting of O, N, S, or a combination thereof;

provided that:

when R is Q, or Q-substituted C1-C6 straight or branched chain alkyl or alkenyl, or Q-substituted C1-C6 straight or branched chain alkyl or alkenyl which is additionally substituted with one or more hydroxyl- or oxo-groups,

then X and Y are not both

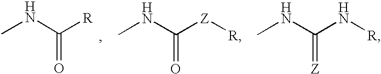

or a combination thereof;

and further provided that:

when R is Q, or Q-substituted C1-C6 straight or branched chain alkyl or alkenyl, or Q-substituted C1-C6 straight or branched chain alkyl or alkenyl which is additionally substituted with one or more hydroxyl- or oxo-groups,

then X and Y are not both:

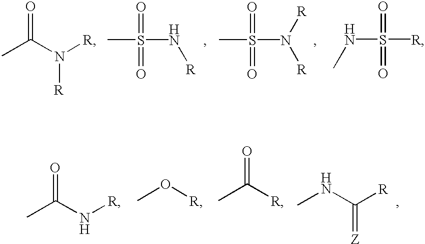

or a combination thereof.

Compounds of formula III are of the following general structure:

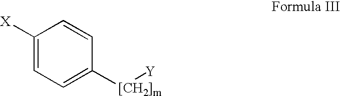

wherein m is 0-3;

X and Y are the same or different, and may independently be:

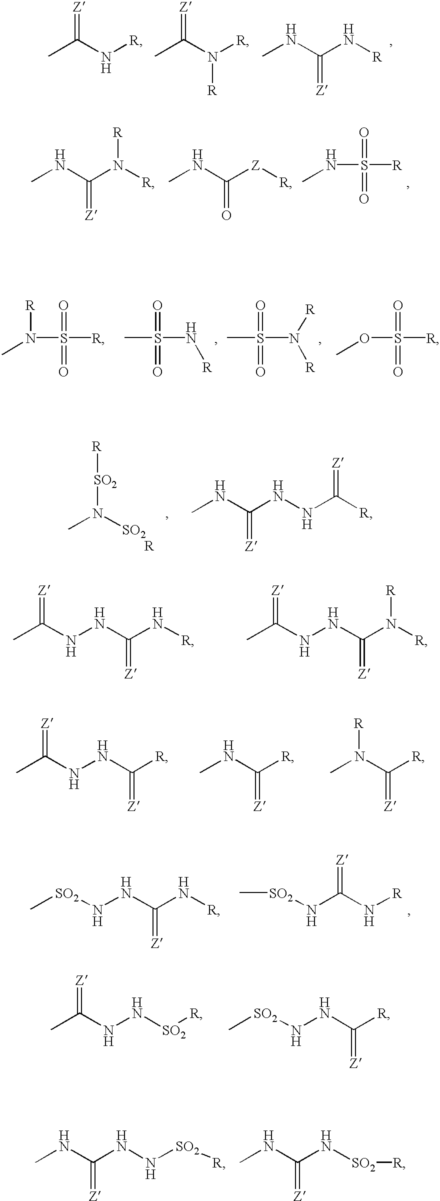

or a combination thereof,

or C₁-C₆ straight or branched chain alkyl, alkenyl, or alkynyl; said alkyl, alkenyl or alkynyl being substituted at one or several positions with Q, and optionally substituted at one or several positions by hydroxyl, mercaptyl, or carbonyl oxygen;

and where Y may further be:

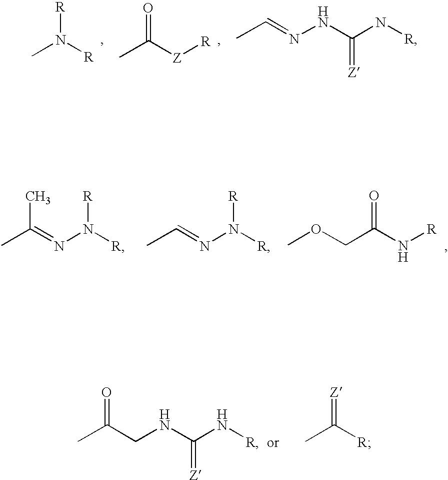

wherein Z′ is O, S, N(CN), CH(NO₂), or N(NO₂);

Z is O or S; and

R may independently be:

or C₁-C₆ straight or branched chain lower alky, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with N, NH, S, SO, or SO₂;

and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; hydroxyl; mercaptyl; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state.

Compounds of formula IV are or the following general structure

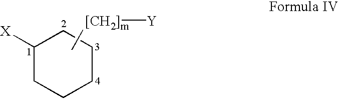

wherein Y is attached at position 2, 3, or 4;

m is 0-3;

the substituent —[CH₂]ₘ—Y is attached at position 2, 3, or 4;

X and Y are the same or different, and may independently be:

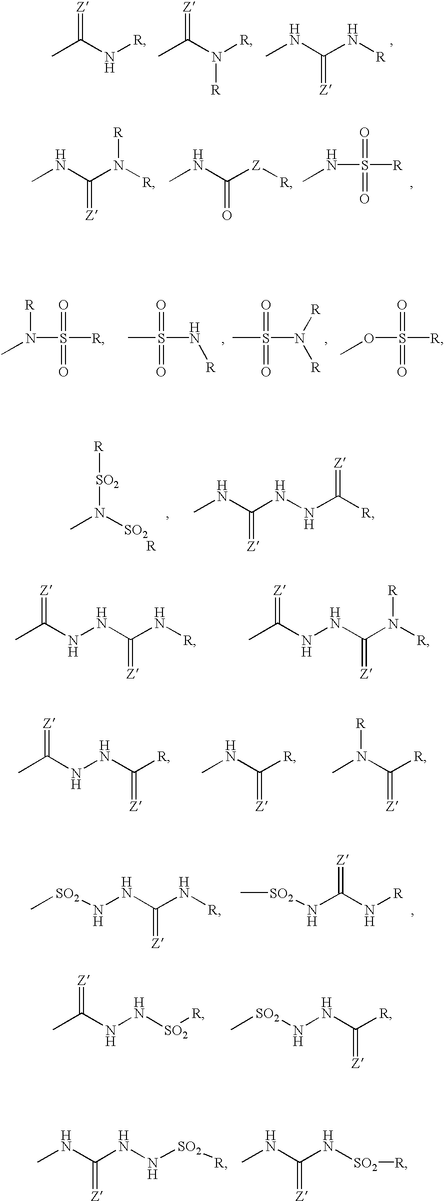

or a combination thereof,

or C₁-C₆ straight or branched chain alkyl, alkenyl, or alkynyl; said alkyl, alkenyl or alkynyl being substituted at one or several positions with Q, and optionally substituted at one or several positions by hydroxyl, mercaptyl, or carbonyl oxygen;

and where Y may further be: Q,

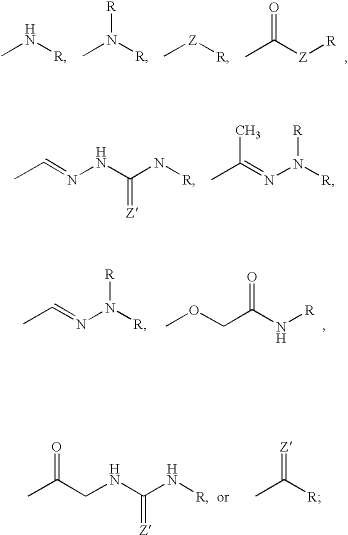

wherein Z′ is O, S, N(CN), CH(NO₂), or N(NO₂);

Z is O or S; and

R may independently be:

Q,

or C₁-C₆ straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;

and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; hydroxyl; mercaptyl; nitro; trifluoromethyl; atninocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state;

provided that:

when R is Q, or Q-substituted C₁-C₆ alkyl or alkenyl, or Q-substituted C₁-C₆ alkyl or alkenyl which is additionally substituted with one or more hydroxyl- or oxo-groups, and

m is 0, and

Y is attached at position 3;

then X and Y are not both

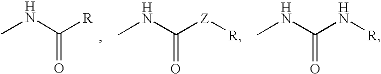

or a combination thereof.

Compounds of formula IVa are of the following general structure:

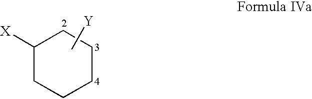

wherein Y is attached at position 2, 3, or 4;

where X and Y are the same or different, and may independently be:

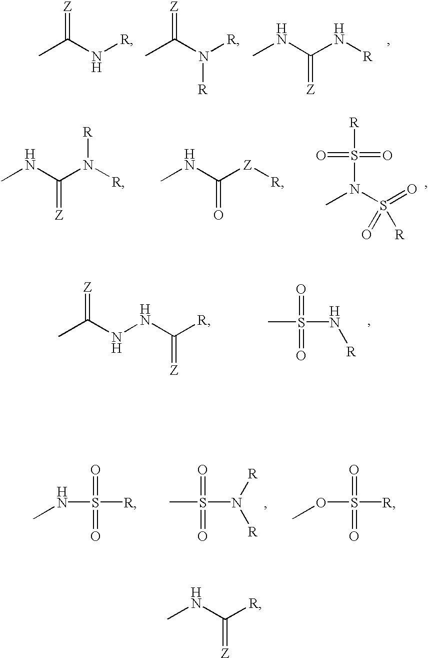

or a combination thereof,

or C1-C6 straight or branched chain lower alkyl, alkenyl, or alkynyl which is substituted at one or several positions with Q, and which further may optionally be substituted at one or several positions by hydroxyl, mercaptyl, or carbonyl oxygen;

and where Y may further be: Q,

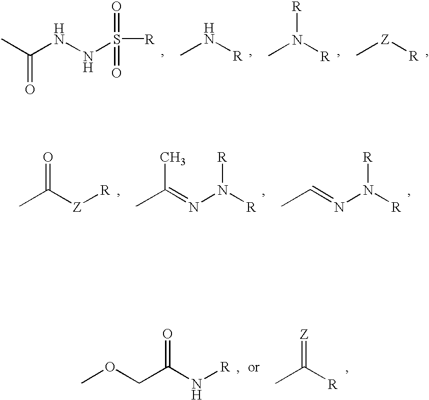

wherein Z is O or S, and

R may independently be:

Q,

or C1-C6 straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;

and wherein Q is a mono-, bi- or tricyclic carbo- or heterocyclic ring which is optionally saturated, partially saturated, or aromatic, and which may optionally be substituted in one or several positions with halo, hydroxyl, mercaptyl, nitro, cyano, trifluoromethyl, C1-C6 straight or branched chain alkyl or -alkenyl, C1-C4 alkoxy or -alkenyloxy, phenoxy, benzyloxy, amino, or acetyl, and wherein the individual ring sizes are 5-6 members, and wherein each heterocyclic ring contains 1-6 heteroatoms selected from the group consisting of O, N, S, or a combination thereof;

provided that:

when R is Q, or Q-substituted C1-C6 straight or branched chain alkyl or alkenyl, or Q-substituted C1-C6 straight or branched chain alkyl or alkenyl which is additionally substituted with one or more hydroxyl- or oxo-groups,

and Y is attached at position 3,

then X and Y are not both

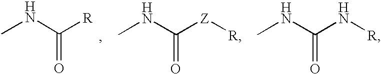

or a combination thereof.

Compounds of formula V are of the following general structure:

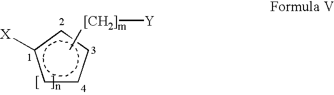

wherein n is 1, forming a central 5-membered carbocyclic ring which is saturated or partially saturated;

m is 0-3;

the substituent —[CH₂]ₘ—Y is attached to said central carbocyclic ring at position 2, 3, or 4;

X and Y are the same or different, and may independently be:

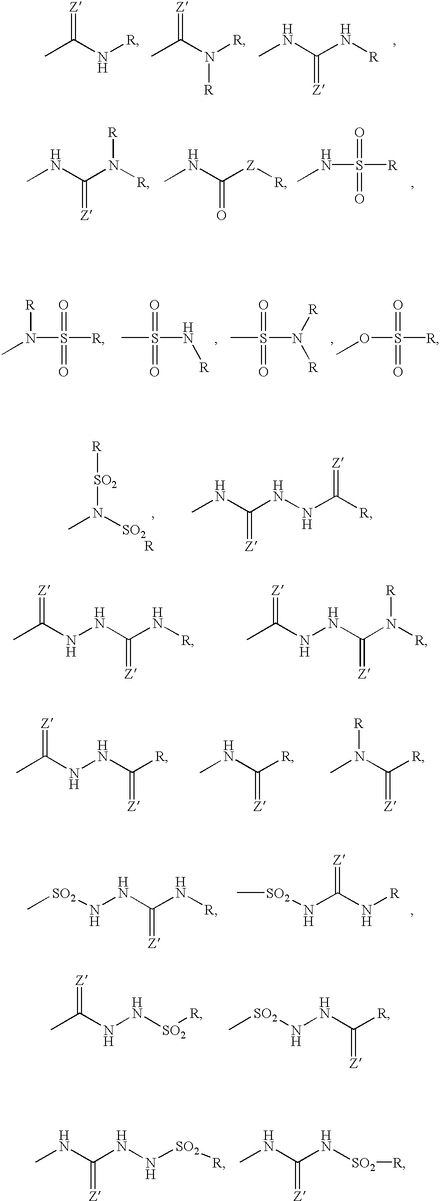

or a combination thereof,

or C₁-C₆ straight or branched chain alkyl, alkenyl, or alkynyl; said alkyl, alkenyl or alkynyl being substituted at one or several positions with Q, and optionally substituted at one or several positions by hydroxyl, mercaptyl, or carbonyl oxygen;

and where Y may further be: Q,

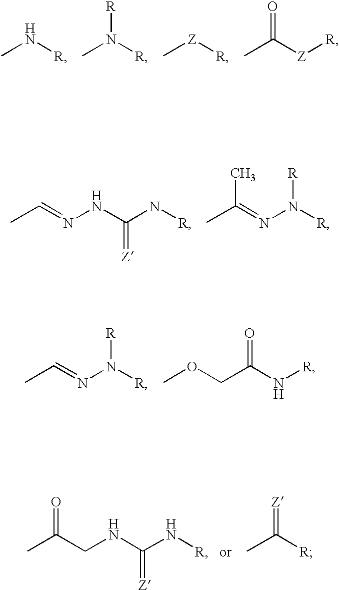

wherein Z′ is O, S, N(CN), CH(NO₂), or N(NO₂);

Z is O or S; and

R may independently be:

Q,

or C₁-C₆ straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;

and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; hydroxyl; mercaptyl; nitro; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state;

Compounds of formula Va are of the following general structure:

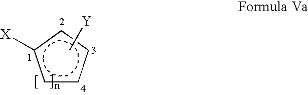

where n is 1, forming a central 5-membered carbocyclic ring which is saturated or partially saturated;

Y is attached to said central carbocyclic ring at position 2, 3, or 4;

X and Y are the same or different, and may independently be:

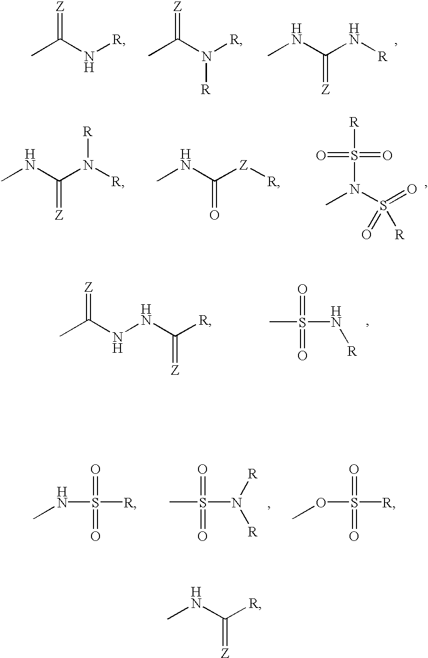

or a combination thereof,

or C1-C6 straight or branched chain lower alkyl, alkenyl, or alkynyl which is substituted at one or several positions with Q, and which further may optionally be substituted at one or several positions by hydroxyl, mercaptyl, or carbonyl oxygen;

and where Y may further be: Q,

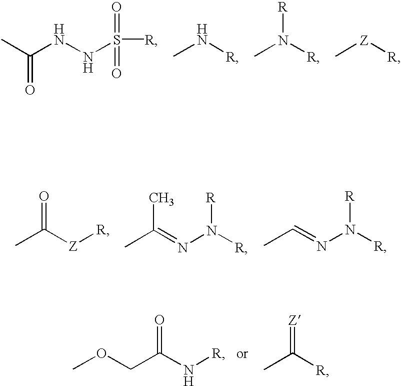

wherein Z is O or S,and

R may independently be:

or C1-C6 straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;

and wherein Q is a mono-, bi- or tricyclic carbo- or heterocyclic ring which is optionally saturated, partially saturated, or aromatic, and which may optionally be substituted in one or several positions with halo, hydroxyl, mercaptyl, nitro, cyano, trifluoromethyl, C1-C6 straight or branched chain alkyl or -alkenyl, C1-C4 alkoxy or -alkenyloxy, phenoxy, benzyloxy, amino, or acetyl, and wherein the individual ring sizes are 5-6 members, and wherein each heterocyclic ring contains 1-6 heteroatoms selected from the group consisting of O, N, S, or a combination thereof.

Compounds of formula VI are of the following general structure:

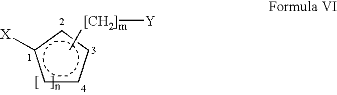

wherein n is 2, forming a central 6 membered carbocyclic ring which is saturated or partially saturated;

m is 0-3;

the substituent —[CH₂]ₘ—Y is attached to said central carbocyclic ring at position 2, 3, or 4;

X and Y are the same or different, and may independently be:

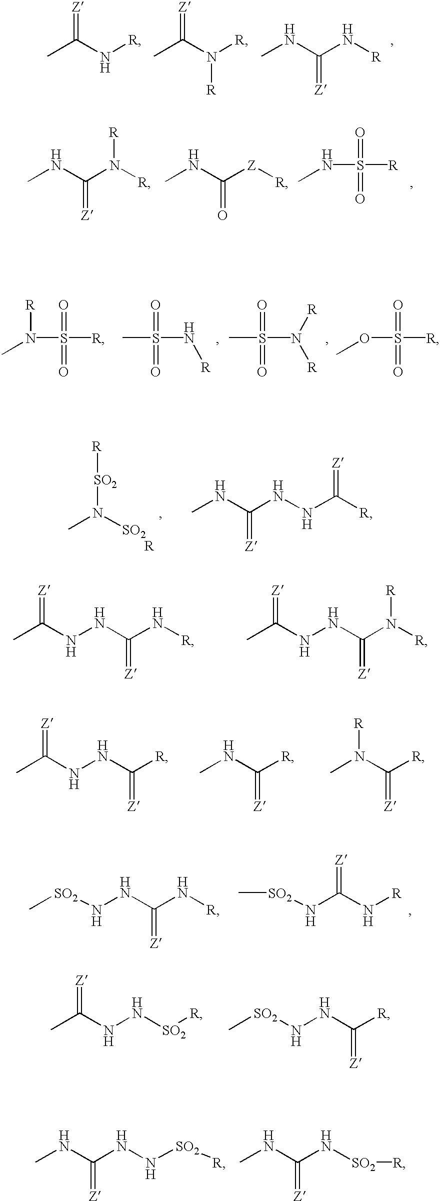

or a combination thereof,

or C₁-C₆ straight or branched chain alkyl, alkenyl, or alkynyl; said alkyl, alkenyl or alkynyl being substituted at one or several positions with Q, and optionally substituted at one or several positions by hydroxyl, mercaptyl, or carbonyl oxygen;

and where Y may further be: Q,

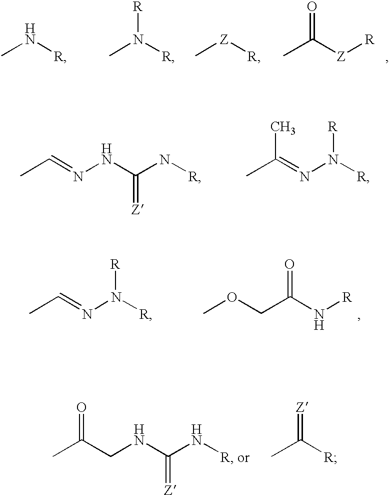

wherein Z′ is O, S, N(CN), CH(NO₂), or N(NO₂);

Z is O or S; and

R may independently be:

Q,

or C₁-C₆ straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;

and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; hydroxyl; mercaptyl; nitro; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state;

provided that:

when R is Q, or Q-substituted C₁-C₆ alkyl or alkenyl, or Q-substituted C₁-C₆ alkyl or alkenyl which is additionally substituted with one or more hydroxyl- or oxo-groups, and

m is 0, and

Y is attached to said central carbocyclic ring at position 3;

then X and Y are not both

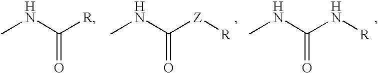

or a combination thereof;

and further provided that:

when R is Q, or methyl monosubstituted with Q, and

m is 0, and

Y is attached to said 6-membered carbocyclic ring at position 2, and said carbocyclic ring is partially saturated,

then X and Y are not both —(CO)—NH—R.

In a preferred embodiment of formula I of this invention, the central carbocyclic ring is a phenyl ring substituted with X and —(CH₂)ₘ—Y at positions 1 and 3.

In another preferred embodiment, the central carbocyclic ring is a cyclohexyl ring substituted with X and —(CH₂)ₘ—Y at positions 1 and 2.

Another preferred embodiment of this invention provides compounds wherein the central carbocyclic ring is a cyclopentane or cyclopentene ring substituted with X and —(CH₂)ₘ—Y at positions 1 and 3.

Further preferred embodiments of this invention provide compounds wherein X and Y are the same, and wherein each R is Q, and wherein each Q may be the same or different.

Yet another preferred embodiment of this invention provides compounds of the general formula VII, as described below:

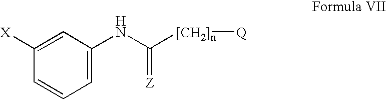

and pharmaceutically acceptable derivatives thereof;

wherein Z is O or S;

n is 2-6;

X is selected from the group consisting of

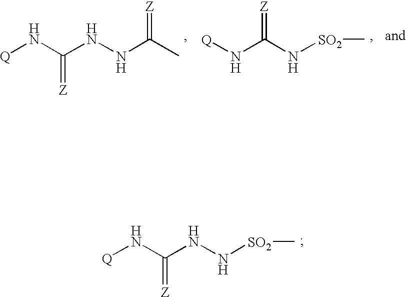

and Q is a 5-6-membered carbo- or heterocyclic ring, which is optionally saturated, partially saturated, or aromatic, and wherein each of one or several heteroatoms, if present, is independently selected from the group consisting of O, N, and S, and wherein Q is optionally substituted at one or several positions with halo or trifluoromethyl.

In this specification, the generic terms “alkyl”, “alkenyl” or “alkynyl” include both straight-chain and branched-chain saturated or unsaturated groups. “Aryl” in terms such as “arylaminocarbonyl” typically means as phenyl, naphthyl, pyrrolyl, pyrrolidinyl, pyridinyl, pyrimidinyl, purinyl, furyl, imidazolyl, quinolinyl, oxazolyl, thiazolyl, pyrazolyl, and thienyl. “Alkyloxy” or “alkoxy” refer to groups such as, for example, methoxy or ethoxy; “alkoxycarbonyl” refers to groups such as, for example, methyl ester or butyl ester; “C₁-C₄ alkylcarbamoyl” and “di(C₁-C₄)alkylcarbamoyl” refer to saturated or unsaturated carbon chains attached via an amide linkage, such as, for example, ethylcarbamoyl or diethylcarbamoyl; “arylaminocarbonyl” refers to groups such as phenylaminocarbonyl (C₆H₅)—NH—CO—; “C₁-C₄ alkanoyl” refers to groups such as, for example, formyl or acetyl; “C₆ alkylamino” refers to groups such as, for example, hexylamino.

All compounds of this invention, can be selected for use from Formulae I-VII. Starting with a particular compound, any of the individual variable groups R, X, Y, Q, Z, Z′, and values for n and m can be selected while one or more of the other variable groups can be modified. For example, in Formula I, the “n” can be set at 2 to select subgroups of related compounds which share a central 6-membered cyclohexane, cyclohexene, or phenyl ring. Any of the subgroups thus obtained can be further divided into additional subgroups of compounds defined by the allowed combinations of X and Y, and by requiring that X and Y are either similar, or different from each other, and by requiring that R be Q, or that R be Q-substituted alkyl, alkenyl, or alkynyl, and that all Q-substituents be the same, or different from each other. This process can be repeated using any one, or a combination of, the variable groups. In this way, one skilled in the art can select and use groups of related compounds or even individual compounds, all within the invention. Many examples are shown below; however, they are merely representative of the scope of changes and modifications possible. One skilled in the art can devise many separate compounds from the description of Formula I alone.

Compounds of Formulae I-VII may be prepared or formulated as a salt or derivative for some uses, including pharmaceutical and tissue or cell culture uses. As used herein, the CyP-binding compounds of this invention are defined to include pharmaceutically acceptable derivatives. A “pharmaceutically acceptable derivative” denotes any pharmaceutically acceptable salt, ester, thioester, or salt of such ester or thioester, of a compound of this invention or any other compound which, upon administration to an animal or human patient, is capable of providing (directly or indirectly) a compound of this invention, or a metabolite or residue thereof, characterized by the ability to bind to a CyP and/or its usefulness in treating or preventing a medical disorder. Examples of medical disorders within the scope of this aspect of the invention are given below. The compounds of the invention can also be part of a composition comprising one or more compounds of Formulae I-VII.

The compounds of the invention can be produced as a mixture of isomers or racemic mixtures or as optically pure compounds. Methods for separating stereoisomers known in the art can also be used to enrich mixtures for one or more compounds. The compositions of the invention may similarly contain mixtures of stereoisomers, mixtures of one or more stereoisomers, or be enriched for one or more stereoisomers. All of these forms are specifically included in this invention and are intended to be included in the claims.

Preferably, compounds of Formulae I-VII selectively bind to a CyP as detected, for example, by a measurable inhibition of the peptidyl-prolyl cis-trans isomerase enzyme activity (PPIase) of CyP. “Selectively bind to a CyP” means the compounds do not possess a significant binding affinity toward a FKBP and/or do not possess a biological activity associated with binding to a FKBP. For example, the IC₅₀ towards FKBP is at or above 500 nM. The skilled artisan is familiar with ways to detect rotamase inhibition in CyP and FKBP. In addition, a number of ways for detecting binding to a CyP are described below.

As is readily apparent from Formulae I-VII, a common substitution pattern exists, wherein at least two carbo- or heterocyclic groups are attached to a central carbocyclic ring by a combination of straight or branched linker chains. This common pattern differs from the approaches previously taken to identify other immunophilin binding compounds or drugs. For example, Holt et al. (Bioorg. Med. Chem. Letters, 4: 315-320 (1994)) discuss a pipecolate, or 1-(1,2-dioxo) 2-carboxylate piperidine containing base structure for binding to FKBP. Similarly, earlier work by the inventors established the relevance of a 1-(1,2-dioxo) 2-carboxylate pyrrolidine containing structure for binding to FKBP (Steiner et al., PNAS 94:2019-2024 (1997)). Presumably, these structures mimic the natural substrate for the peptidyl-prolyl-isomerase (PPIase) activity, a proline-containing fragment of a protein. In a protein, the amino acid proline corresponds to a 1,2-substituted pyrrolidine structure. Prior work has generally incorporated that structure. However, Formulae I-VII do not correspond to a 1,2-substituted pyrrolidine structure. Yet, as demonstrated here, compounds of this formula possess important bioactive and biochemical functions.

The body of work related to analogues of cyclosporin A, FK-506, and rapamycin further distances the compounds of this invention from prior work. (See, for example, U.S. Pat. Nos. 5,767,069, 5,284,826, 4,703,033, and 5,122,511.) These analogues typically possess a cyclic peptide structure.

In another aspect, the invention relates to methods for binding non-peptidic compounds to cyclophilin-type immunophilins. While the present invention is not bound by this theory, it is hypothesized that binding results in an “immunophilin:drug” complex, which is considered to be the active agent in the in vivo immunosuppressive and neurotrophic activities of PPIase inhibitors (Hamilton and Steiner, J. of Med. Chem. 41:5119-5143 (1998); Gold, Mol. Neurobiol. 15:285-306 (1997)). Whether or not the complex acts for any or all the therapeutic actions of these PPIase inhibitors, focusing on the immunophilin:drug interaction has led to the discovery a number of new drug compounds. Accordingly, methods of using compounds, such as those of Formulae I-VII, to create an immunophilin:compound complex, or a CyP:compound complex, provide an important aspect of this invention. This aspect can be exploited, for example, in methods where the compound, or a mixture comprising one or more of the compounds of the invention, or a pharmaceutical composition comprising one or more of the compounds of the invention, is administered to cells in culture or to an animal.

While the immunophilin:compound complex has beneficial effects in vivo and in vitro in cultured cells, numerous other uses for binding the compounds to an immunophilin exist. For example, further in vitro binding experiments can be used to identify and purify cellular components that interact with the immunophilin complex in a cell-free environment, as would be the case where an affinity chromatography column or matrix bearing the compound is reacted with a CyP, and cellular or tissue extracts containing a CyP are passed over the column or matrix.

Thus, the invention also provides methods for forming immunophilin:compound or CyP:compound complexes as well as the complexes themselves. To form these complexes, the compounds can contact an immunophilin or CyP protein in vivo, in cell or tissue culture, or in a cell-free preparation. In preferred embodiments, the compound contacts a human CyP protein, such as one or more of CyP A, B, C, D, or −40. The CyP protein can be native to the cell or organism, produced via recombinant DNA, produced by other manipulations involving introduced genetic material, or produced by synthetic means. Furthermore, chimeric proteins possessing immunophilin domains that function to bind immunophilin ligands can also be used to form a protein:compound complex. The formation of the CyP:compound, immunophilin:compound, or protein:compound complex need not be irreversible.

The binding of a compound to a CyP can be detected in a number of ways, including PPIase inhibition assay, affinity chromatography, in vivo neuroprotection or neuroregeneration activity assay, in vitro neurotrophic activity assay, in vitro mitochondrial swelling assay, in vivo ischemia/reperfusion injury model assays, or by any of the activities in neuronal cells or cells of the nervous system described below, in the examples, or in the cited references.

The invention also provides compositions comprising at least one compound of Formulae I-VII. The compositions may comprise one or more pharmaceutically acceptable carriers, excipients, or diluents. These compositions, or the compounds themselves or mixtures of them, can be administered to an animal. Administration can be one method to allow the compound to contact a CyP within the animal. As one skilled in the art would recognize, various routes of administration are possible. Exemplary routes are specifically described in the detailed description below.

The compounds of Formulae I-VII, or compositions comprising them, can function to regenerate nerve cells, promote neurite outgrowth, and protect nerves from otherwise damaging treatments or conditions. Thus, the compounds and compositions of this invention are useful in the diagnosis, cure, mitigation, treatment, or prevention of neurological disorders in animals, including humans, and in animals (including humans) exposed to neurodegenerative agents or having damaged nervous system cells. Such neurological disorders, when present in an animal, including humans, can be neurodegenerative disorders, neuropathic disorders, neurovascular disorders, traumatic injury of the brain, spinal cord, or peripheral nervous system, demyelinating disease of the central or peripheral nervous system, metabolic or hereditary metabolic disorder of the central or peripheral nervous system, or toxin-induced- or nutritionally related disorder of the central or peripheral nervous system. When present in a human, a neurodegenerative disorder can be, for example, Parkinson's disease, Alzheimer's disease, amyotrophic lateral sclerosis (ALS), Huntington's disease, cerebellar ataxia, or multisystem atrophy including, for example, olivopontocerebellar degeneration, striatonigral degeneration, progressive supranuclear palsy, Shy-Drager syndrome, spinocerebellar degeneration and corticobasal degeneration. A demyelinating disease can be, for example, multiple sclerosis, Guillain-Barré syndrome, or chronic inflammatory demyelinating polyradiculoneuropathy. A neurovascular disorder can be global cerebral ischemia, spinal cord ischemia, ischemic stroke, cardiogenic cerebral embolism, hemorrhagic stroke, lacunar infarction, multiple infarct syndromes including multiple infarct dementia, or any disorder resulting in ischemia or ischemia/reperfusion injury of the central nervous system. Traumatic injury of the central or peripheral nervous system can be, for example, concussion, contusion, diffuse axonal injury, edema, and hematoma associated with craniocerebral or spinal trauma, or axonal or nerve sheath damage associated with laceration, compression, stretch, or avulsion of peripheral nerves or plexi, and further includes damage to central nervous tissue or peripheral or visceral nervous tissue caused during surgery, such as damage to the major pelvic ganglion and/or cavernous nerve caused during prostate surgery. A neuropathic disorder can be, for example, diabetic neuropathy, uremic neuropathy, neuropathy related to therapy with drugs such as phenytoin, suramin, taxol, thalidomide, vincristine or vinblastine; or neuropathy/encephalopathy associated with infectious disease, such as, for example, encephalopathy related to HIV, rubella virus, Epstein-Barr virus, herpes simplex virus, toxoplasmosis, prion infection. A metabolic disorder of the central nervous system can be, for example, status epilepticus, hypoglycemic coma, or Wilson's disease.

The following detailed description should not be taken as a limitation on the scope of the invention, and all embodiments and examples given are merely illustrative of the invention. Additional aspects of the invention can be devised by reference to this disclosure as a whole in combination with the references cited and listed throughout and at the end of the specification and the knowledge of one skilled in the art. All of the references cited and listed can be relied on, in their entirety, to allow one to make and use these additional aspects of the invention.

### DETAILED DESCRIPTION OF THE INVENTION

One skilled in the art can refer to general reference texts for detailed descriptions of known techniques discussed herein or equivalent techniques. These texts include Current Protocols in Molecular Biology [Ausubel, et al., eds., John Wiley & Sons, N.Y., and supplements through May 2001], Current Protocols in Immunology [Coligan, et al., eds., John Wiley and Sons, N.Y., and supplements through May 2001], and Current Protocols in Pharmacology [Enna et al., eds., John Wiley & Sons, N.Y., and supplements through May 2001) for example, each of which are specifically incorporated by reference in their entirety. These texts can also be referred to in making or using an aspect of the invention.

As noted above, cyclosporin A was the first compound identified to bind a CyP. Based on the cyclic structure of cyclosporin A, a number of large, usually cyclic peptides were developed as immunosuppressive compounds that bind CyP. Now, unexpectedly, the inventors have found a non-peptidic class of CyP binding compounds with activity in neuronal cells.

The following compounds are representative of those tested:

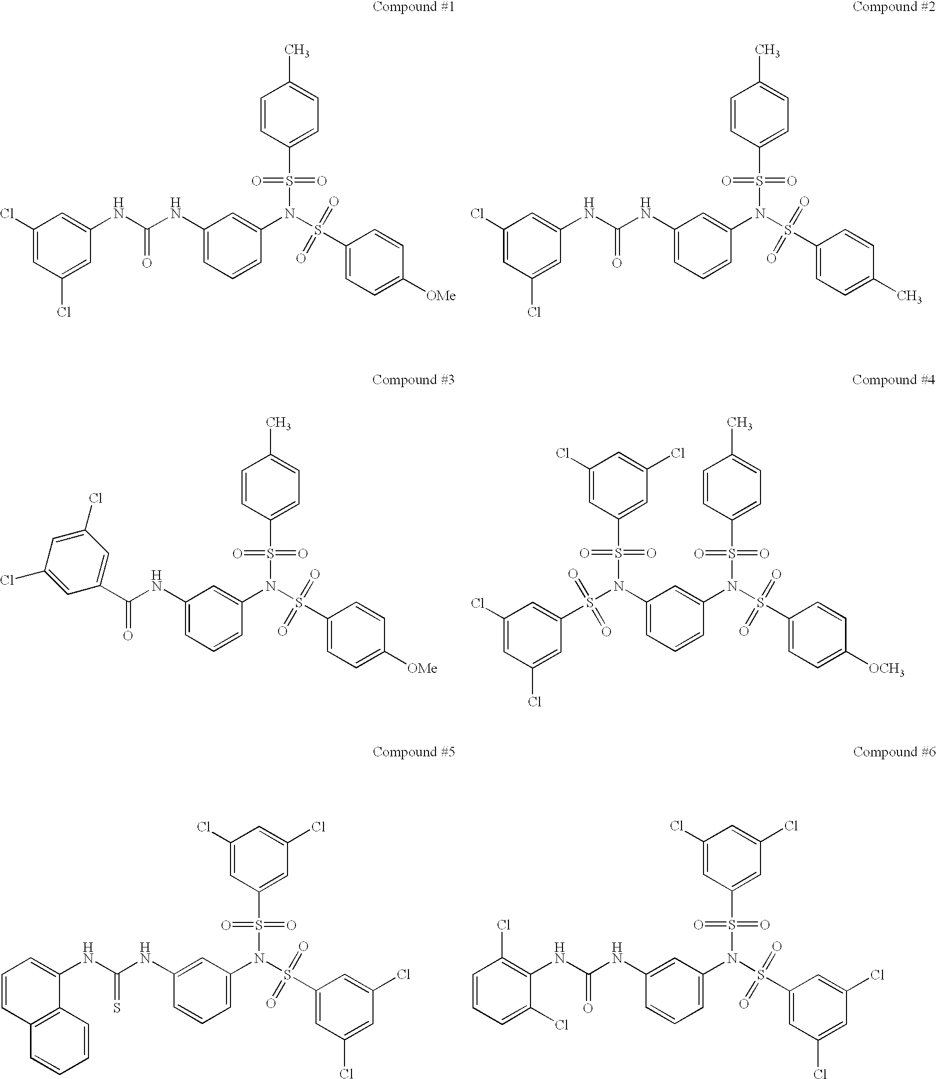

Preparation of Compounds of the Invention

The compounds of the invention can be prepared by a number of synthetic routes. The examples below detail Schemes I to XIX for the preparation of specific compounds. However, one skilled in the art can modify the steps, reactants, and reaction conditions in the examples and schemes to arrive at numerous examples of compounds of the invention. In addition, if particular stereoisomers or mixtures are desired, the starting materials and/or reactants in the preparatory scheme can be selected and used accordingly. Alternatively or in addition, particular intermediates can be purified or enriched by chromatographic or enzymatic methods, or by manipulating reaction conditions or selective crystallization, to generate particular final products or mixtures. One skilled in the art is familiar with numerous methods to selectively produce or enrich for desired stereoisomers or mixtures. All of the compounds of the examples, including the intermediates, are specifically included in the compounds of the invention and can be used in the methods of the invention.

The compounds of the invention may be prepared as salts or derivatives.

Various salts and derivatives are known in the art and a non-limiting list of possible choices includes acid salts: acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, mesylate, dimesylate, pamoate, pectinate, persulfate, 3-phenylpropionate, phosphates, picrate, pivalate, propionate, succinate, sulfates, tartrate, thiocyanate, tosylate, and undecanoate. Base salts may include: amine salts, ammonium salts, alkali metal salts such as sodium and potassium salts, alkaline earth metal salts such as calcium and magnesium salts, salts with organic bases such as dicyclohexylamine salts, N-methyl-D-glucosamine, and salts with amino acids, for example arginine or lysine. Nitrogen-containing groups of the compound can be quaternized with agents as: alkyl halides, for example methyl, ethyl, propyl, and butyl chlorides, bromides, or iodides; dialkyl sulfates, for example dimethyl, diethyl, dibutyl and diamyl sulfates, long chain halides, for example decyl, dodecly, lauryl, myristyl, or stearyl chlorides, bromides, or iodides; and aralkyl halides, for example benzyl and phenethyl bromides, chlorides, or iodides. The skilled artisan is familiar with methods for producing and testing any suitable salt or derivative. (See, for example, Remington's Pharmaceutical Sciences, Mack Publishing Co., Easton, Pa., 18^{th} Edition, specifically incorporated herein by reference.)

Activity in Neuronal or Nervous System Cells

In general, activity in the nervous system for a particular compound can be identified by assaying for the ability to promote neurite outgrowth, protect neurons from damage by chemical treatments, promote the growth of neurons or neuronal cells, recover lost or damaged motor, functional or cognitive ability associated with nervous tissue or organs of the nervous system, or regenerate neurons. These activities can be useful in treating, diagnosing, or prognosing a number of human disease conditions, including, but not limited to, the neurological conditions described above, as well as disorders of the retina and optic nerve, vestibulocochlear disorders, and erectile dysfunction related to nerve damage caused during prostate surgery.

A number of animal model assays and cell culture assays have been developed and can be relied on for their clinical relevance to disease treatments, including the human diseases noted above. Each of the following references can be used as a source for these assays, and all of them are specifically incorporated herein by reference in their entirety for that purpose: Steiner, et al., PNAS 94: 2019-2024 (1997); Hamilton, et al., Bioorgan. Med.Chem.Lett. 7:1785-1790 (1997); McMahon, et al, Curr. Opin. Neurobiol. 5:616-624 (1995); Gash, et al., Nature 380:252-255 (1996); Gerlach, et al., Eur. J. Pharmacol.-Mol. Pharmacol. 208:273-286 (1991); Apfel, et al., Brain Res. 634:7-12 (1994); Wang, et al., J. Pharmacol. Exp. Therap. 282:1084-1093 (1997); Gold, et al., Exp. Neurol. 147:269-278 (1997); Hoffer et al., J. Neural Transm. [Suppl.] 49:1-10 (1997); Lyons, et al., PNAS 91:3191-3195 (1994); Yoshimoto and Siesjö, Brain Res., 839, pp. 283-91 (1999); Kondo et al., Neurochem Res., 24, pp. 9-13 (1999); Friberg et al., J Neurosci., 18, pp. 5151-9 (1998); Sullivan et al., Exp Neurol., February 2000; 161, 631-7 (2000).

Preferred methods for detecting neuronal activity include a neuroprotective assay, for example an organotypic slice culture of the spinal cord, in which a compound is tested for the ability to protect against treatment causing glutamate neurotoxicity. Sensory neuronal cultures of the dorsal root ganglia (DRG) can also be assayed for neurite outgrowth, an assay for neurotrophic activity. Cultured cells are treated with a compound of the invention and later assayed for the presence of new neurite fibers. The compounds can also be tested for their ability to inhibit the mitochondrial permeability transition by measuring large amplitude mitochondrial swelling of freshly isolated rat liver mitochondria in a spectrophotometric assay [Broekemeier, et al., J. Biol. Chem. 264: 7826-7830 (1989)].

The compounds of the present invention can also be assayed for their in vivo potency and efficacy using a common mouse model of a neurodegenerative disorder: Mice can be treated orally or subcutaneously, for example, with the compounds of the present invention, and subsequently be subjected to MPTP-treatment. MPTP (N-methyl-4-phenyl-1,2,3,6-tetrahydropyridine) is a systemically available neurotoxin that selectively destroys the dopaminergic neurons of the ventral midbrain as well as their forebrain projections. One skilled in the art is familiar with methods for assessing the integrity of the midbrain-forebrain projection in MPTP-lesioned mice that were treated with the compounds of this invention, and a relative preservation of the nerve fibres, nerve terminals, or of the dopaminergic cell bodies in the ventral midbrain, would be indicative of the relative neuroprotective efficacy of the compounds of this invention. In the assays here exemplified, immunohistochemistry can aid in the visualization and quantitation of neurites, terminals and cell bodies.

The compounds of the invention can also be used to promote the establishment or maintenance of tissue or cell cultures. Similar to the use for promoting neuronal cell growth, the compounds can be added to primary, transformed, or established cell cultures. Particularly in the case of neuronal cells, the compounds can induce growth in culture and extend the culture lifetime of cells.

Binding to CyP and Other Uses

A recognized method for assessing the affinity of the compound to cyclophilin is the rotamase inhibition assay. For this purpose, the following references are specifically incorporated by reference and can be relied on to make assays of rotamase inhibition: Fischer, et al., Biomed. Biochem. Acta 43:1101-1112 (1984); Kofron, et al., Biochem. 30:6127-6134 (1991); Kofron et al., J. Am. Chem. Soc. 114:2670-2675 (1992); Harrison et al., Biochem. 29:3813-3816 (1990); Lang et al., Nature 329:268-270 (1987); Mucke et al., Biochem. 31:7848-7854 (1992); Schonbrunner et al., J. Biol. Chem.266:3630-3635 (1991); Hsu et al., J. Am. Chem. Soc. 112:6745-6747 (1990); and Justice et al., Biochem. Biophys. Res. Commun. 171:445-450 (1990).

Additional uses for the compounds, which may or may not relate to CyP binding, are also included in the methods of the invention. For example, the compounds are useful to promote hair growth, and to prevent or retard hair loss. In murine models which mimic human premature hair follicle regression or human chemotherapy-induced hair loss, topical application of CsA was found to induce and maintain hair growth, and topical or systemic administration of CsA was found to protect from hair loss induced by cancer chemotherapeutic agents (see, e.g., Maurer, et al. Am. J. Pathol. 150(4):1433-41 (1997); Paus, et al., Am. J. Pathol. 144, 719-34 (1994)). It has been speculated that initiation of hair growth by CsA is unrelated to immunosuppression (Iwabuchi, et al., J. Dermatol. Sci. 9, 64-69 (1995)). The compounds of the invention are useful in preventing or retarding hair loss in patients undergoing therapy with doxorubicin, carboplatin, cisplatin, cyclophosphamide, dactinomycin, etoposide, hexamethamelamine, ifosfamide, taxol, vincristine, bleomycin, 5-fluorouracil, and other agents useful in the therapy of cancer. The compounds of the invention are further useful in promoting hair growth in patients suffering from hair loss caused by one or a combination of the aforementioned chemotherapeutic agents. The compounds of the invention are further useful in the prevention of hair loss, and in the promotion of hair growth, in patients undergoing radiation therapy, and in patients suffering from alopecia areata, androgenetic alopecia/male pattern baldness, anagen effluvium, trichotillomania, traction alopecia, telogen effluvium, and hair loss induced by drugs such as, for example, methotrexate, nonsteroidal anti-inflammatory drugs, or beta blockers.

For these purposes, the compounds may be administered as part of pharmaceutical or cosmetic compositions, singly, in combination with other compounds of the invention, in combination with other hair growth-promoting or hair-loss preventing agents, or in combination with one or several other active agents such as, for example, antibiotic agents, antidandruff agents, and anti-inflammatory agents.

The compounds of the invention are also useful to treat or affect mitochondrial disorders, metabolic disorders, diabetes, or vision loss. The mitochondrion is increasingly being recognized as an important mediator of cell death in hypoxia, ischemia, and chemical toxicity. Disruption of the mitochondrial transmembrane potential is observed before other features of apoptosis (e.g. generation of reactive oxygen species or internucleosomal DNA fragmentation (“laddering”)) become detectable. This applies to many different models of apoptosis induction, such as, for example, NGF-deprivation of cultured sympathetic neurons, dexamethasone-induced lymphocyte apoptosis, programmed lymphocyte death, activation-induced programmed cell death of T cell hybridomas, and tumor necrosis factor-induced death of lymphoma cells. [Marchetti, P., et al., J. Exp. Med. 184, 1996, 1155-1160]. Breakdown of mitochondrial transmembrane potential in proapoptotic cells has been attributed to the formation of an unspecific high conductance channel—the mitochondrial permeability transition pore—which leads to an increased permeability of the inner mitochondrial membrane to small molecular weight solutes. The ensuing release of intramitochondrial ions, influx of solutes, uncoupling of oxidative phosphorylation, and loss of metabolic intermediates accompanies large amplitude mitochondrial swelling and a depletion of cellular energy stores [see, e.g., Lemasters, J. J. et al., Mol. Cell. Biochem. 174 (1997) 159-165]. Importantly, CsA and non-immunosuppressive peptidic CsA analogues have been described to potently block pore conductance and inhibit the onset of the mitochondrial permeability transition [Broekemeier, K. M., et al., J. Biol. Chem. 264 (1989) 7826-7830; Zamzami, M., et al., FEBS Lett. 384 (1996) 53-7]. The mitochondrial permeability transition pore forms under calcium overload conditions such as occur in ischemia/reperfusion injury, and it has been found that administration of CsA and/or non-immunosuppressive peptidic CsA analogues, by blocking the permeability transition pore, leads to significant protection in experimental models of cerebral stroke [Matsumoto, S., et al., J. Cereb. Blood Flow Metab. 19 (1999) 736-41], cardiac ischemia [Griffiths, E. J. and Halestrap, A. P., J. Mol. Cell Cardiol. 25 (1993) 1461-1469], and hepatic ischemia/reperfusion injury [Leducq, N., et al., Biochem. J. 336 (1998) 501-6]. The compounds of the invention are useful in blocking the mitochondrial permeability transition pore; inhibiting breakdown of mitochondrial metabolism in cells which undergo oxidative stress, calcium overload, excitotoxic or hypoglycemic injury both in vitro and in vivo; inhibiting mitochondrial swelling; inhibiting, both in vivo and in vitro, breakdown of energy metabolism and cell death of mammalian cells following either physiological induction of programmed cell death through signal molecules such as, for example, tumor necrosis factor, or following physiological stress related to hypoxia, hypoglycemia, excitotoxic insult, or calcium overload. The inventive compounds are useful in preventing or delaying cell death in large scale/commercial scale cell culture. The compounds of the invention are further useful in the diagnosis, cure, mitigation, treatment, or prevention of ischemic injury or ischemia/reperfusion injury, such as mesenteric infarction, bowel ischemia, hepatic infarction or ischemia/reperfusion injury, renal infarction, splenic infarction, or cardiac ischemia or ischemia/reperfusion injury related, for example, to angina pectoris, congestive heart failure, or myocardial infarction. Additional uses of the compounds of the invention include applications in the diagnosis, cure, mitigation, treatment, or prevention of Reye's syndrome; ophthalmic disorders such as glaucoma, ischemic or vascular retinopathies, or degeneration of the photoreceptor cell layer. The invention also provides a method of preventing or reducing tissue damage of organs used in organ transplantation surgery, comprising contacting said organs with a compound of Formulae I-VII.

CsA and its non-immunosuppressive peptidic analogues have also been found to potently inhibit the growth of pathogenic protozoan parasites, such as Cryptosporidium parvum, Plasmodium falciparum, Plasmodium vivax, Schistosoma spec., and Toxoplasma gondii [Perkins, et al., Antimicrob. Agents Chemother. 42: 843-848 (1998)]. Although antiprotozoan activity appears not to be correlated with immunosuppressive or PPIase inhibitory activity [Bell, et al., Biochem. Pharmacol. 48:495-503 (1994); Khattab, et al., Exp. Parasitol. 90:103-109 (1998)], the protozoan cyclophilin, complexed to CsA or its nonimmunosuppressive analogues, has been proposed to play an active role in mediating the antiparasitic effects of peptidic cyclophilin ligands [Berriman and Fairlamb, Biochem. J. 334:437-445 (1998)].

CyA and its non-immunosuppressive analogues also inhibit reproduction of filarial parasites in vivo with a potency unrelated to their immunosuppressive activity and their activity against Plasmodium [Zahner and Schultheiss, J. Helminthol. 61:282-90 (1987)], and have been shown to exert direct antihelmintic effects [McLauchlan, et al., Parasitology 121:661-70 (2000)].

The compounds of this invention are useful in the diagnosis, cure, mitigation, treatment, or prevention of infections with pathogenic protozoan or helmintic parasites in animals, including humans. In humans, the present compounds find application in the treatment of conditions such as, for example, malaria, river blindness, lymphatic filariasis, intestinal roundworm infection, tapeworm infection, pinworm infection, toxoplasmosis, leishmaniasis, trypanosomiasis, and bilharzia.

The compounds of this invention are also useful in affecting the viral replication process of the HIV-1 virus. The infectivity of the HIV-1 virus is believed to depend critically upon an interaction of the viral Gag polyprotein capsid complex with host Cyclophilin A. [Streblow et al. Virology 1998: 245, 197-202; Li et al. J. Med. Chem. 2000: 43,1770-9]. The compounds of this invention can function to inhibit or disrupt the interaction of human host CyPA with HIV-1 Gag proteins, to decrease or eliminate the infectivity of the HIV-1 virus, to treat or prevent infection of humans with the HIV-1 virus, and to treat or prevent acquired immune deficiency syndrome (AIDS) associated with HIV-1 infection. The compounds of this invention are further useful in the diagnosis, treatment, cure, mitigation or prevention of infections with strains of the human immunodeficiency virus other than HIV-1, and of infections caused by other pathogenic viruses, such as influenza viruses.

Pharmaceutical Formulations and Routes of Administration

The compounds of the invention have utility in pharmacological compositions for the treatment and prevention of various neurological, ischemic, and inflammatory disorders or for various in vitro and cell culture treatments. The compounds also have utility in pharmacological compositions for the treatment and prevention of HIV-infection, promotion of hair growth, immunosuppression, mitochondrial disorders, traumatic injury to nervous tissue, or conditions associated with retinal and optic nerve damage. The compounds of the invention may be prepared as a salt or derivative, as described above.

A compound of the invention can be administered to an animal or human patient by itself or in pharmaceutical compositions where it is mixed with suitable carriers or excipients, at doses to treat or ameliorate various conditions. The compounds according to the present invention preferably have sufficient stability, potency, selectivity, solubility and availability to be safe and effective in treating diseases, injuries and other abnormal conditions or insults to the central nervous system, the peripheral nerves, and other organs. A therapeutically effective dose refers to that amount of the compound sufficient to effect an activity in a nerve or neuronal cell, to produce a detectable change in a cell or organism, or to treat a disorder in a human or other mammal. The word “treat” in its various grammatical forms as used in relation to the present invention refers to preventing, curing, reversing, attenuating, alleviating, minimizing, suppressing, ameliorating or halting the deleterious effects of a disease state, disease progression, injury, wound, ischemia, disease causative agent (e.g., bacteria, protozoans, parasites, fungi, viruses, viroids and/or prions), surgical procedure or other abnormal or detrimental condition (all of which are collectively referred to as “disorders,” as will be appreciated by the person of skill in the art). A “therapeutically effective amount” of a compound according to the invention is an amount that can achieve effective treatment, and such amounts can be determined in accordance with the present teachings by one skilled in the art.

The methods of the present invention comprise (i.) administration of a compound of Formulae I-VII, where the compound is itself therapeutically active in the treatment of the targeted medical condition, or (ii.) administration of a prodrug of a compound of Formulae I-VII, wherein such prodrug is any compound which is capable of undergoing metabolic conversion to a compound of Formulae I-VII following administration, or (iii.) administration of a compound of Formulae I-VII where the compound is capable of undergoing metabolic conversion to a metabolite following administration, and where the metabolite is therapeutically active in the treatment of the targeted medical condition, or (iv.) administration of a metabolite of a compound of Formulae I-VII, where the metabolite is therapeutically active in the treatment of the targeted medical condition. Thus, the use of a compound of Formulae I-VII in the methods of the present invention explicitly includes not only the use of the compound itself, but also the modifications ii, iii, and iv discussed in this paragraph, and all such modifications are explicitly intended to be within the scope of the following claims.

Therapeutically effective doses may be administered alone or as adjunctive therapy in combination with other treatments. Techniques for the formulation and administration of the compounds of the instant application may be found in Remington's Pharmaceutical Sciences, Mack Publishing Co., Easton, Pa., 18^{th} edition (1990).

Suitable routes of administration may, for example, include oral, rectal, transmucosal, buccal, or intestinal administration; parenteral delivery, including intramuscular, subcutaneous, intramedullary injections, as well as intrathecal, direct intraventricular, intravenous, intraperitoneal, intranasal, or intraocular injections, and optionally in a depot or sustained release formulation. Furthermore, one may administer the agent of the present invention in a targeted drug delivery system, for example in a liposome coated with an antibody. The liposomes will be targeted to and taken up selectively by cells expressing the appropriate antigen.

The pharmaceutical compositions of the present invention may be manufactured in a manner that is itself known, e.g., by means of conventional mixing, dissolving, emulsifying, encapsulating, entrapping, or lyophilizing processes. Pharmaceutical compositions for use in accordance with the present invention thus may be formulated in conventional manner using one or more physiologically acceptable carriers comprising excipients and auxiliaries, which facilitate processing of the active compounds into preparations, which can thus be used pharmaceutically.

For injection, the agents of the invention may be formulated in aqueous solutions, preferably in physiologically compatible buffers, such as Hank's solution, Ringer's solution, or physiological saline buffer. For transmucosal or buccal administration, penetrants appropriate to the barrier to be permeated may be used in the formulation. Such penetrants are known in the art.

For oral administration, the compounds can be formulated readily by combining the active compounds with pharmaceutically acceptable carriers, well known to those in the art. Such carriers enable the compounds of the invention to be formulated as tablets, pills, capsules, liquids, quick-dissolving preparations, gels, syrups, slurries, suspensions and the like, for oral ingestion by a patient to be treated. Pharmaceutical preparations for oral use of the compounds of this invention can be obtained by employing a solid excipient, optionally grinding a resulting mixture, and processing the mixture of granules, after adding suitable auxiliaries, if desired, to obtain tablets. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

In general, the pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. If desired, disintegrating agents may be added, such as the cross-linked polyvinyl pyrrolidone, agar, or alginic acid or a salt thereof such as sodium alginate or a number of others disintegrants (see, for example, Remington's Pharmaceutical Sciences, Mack Publishing Co., Easton, Pa., 18^{th} edition (1990)).

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, pressurized air, or other suitable gas or mixture. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions. Alternatively, the active ingredient may be in powder form for reconstitution with a suitable vehicle, e.g., sterile pyrogen-free water, before use.

The compounds may also be formulated in rectal compositions such as suppositories, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compounds of the invention may further be formulated in pharmaceutical or cosmetic compositions for topical application to the skin in the form of an aqueous, alcoholic, aqueous/alcoholic or oily solution, or of a dispersion of the lotion or serum type, of an emulsion having a liquid or semi-liquid consistency of the milk type, obtained by dispersion of a fatty phase in an aqueous phase (O/W) or vice versa (W/O), or of a suspension or of an emulsion with a soft consistency of the aqueous or anhydrous gel, foam or cream type, or, alternatively, of microcapsules or microparticles, or of a vesicular dispersion of ionic and/or nonionic type, or may further be administered in the form of an aerosol composition comprising a pressurized propellent agent. The compounds of the invention can also be formulated into various compositions for hair care and, in particular, shampoos, hair-setting lotions, treating lotions, styling creams or gels, dye compositions (in particular oxidation dyes), optionally in the form of color-enhancing shampoos, hair-restructuring lotions, permanent-wave compositions, and the like. Pharmaceutical or cosmetic compositions comprising compounds of the invention can also contain additives and adjuvants which are conventional in the cosmetics field, such as gelling agents, preservatives, antioxidants, solvents, fragrances, fillers, screening agents, odor absorbers and colorants. The amounts of these different additives and adjuvants are those typically employed in the cosmetics field and range, for example, from 0.01% to 20% of the total weight of the composition, preferably 0.1% to 10%, and more preferably 0.5% to 5%. In addition to one or several compounds of the invention, compositions for topical application may further contain additional agents already known in the art to promote hair growth or to prevent or retard hair loss, such as, without limitation, tocopherol nicotinate, benzyl nicotinate or 2,4-diamino-6-piperidinopyrimidine 3-oxide, or may contain other active agents such as antibacterial agents, antiparasitic agents, antifungal agents, antiviral agents, anti-inflammatory agents, antipruriginous agents, anaesthetic agents, keratolytic agents, antiseborrhoeic agents, antidandruff agents, or antiacne agents, all of which are well-known in the cosmetic and pharmaceutical arts. The cosmetic or pharmaceutical compositions according to the invention can be topically applied onto the alopecic areas of the scalp and skin of an individual and optionally maintained in contact for a number of hours and optionally rinsed. It is possible, for example, to apply the composition containing an effective amount of at least one compound of the invention in the evening, to retain the composition in contact overnight and optionally to shampoo in the morning. These applications can be repeated daily for one or a number of months, depending on the particular individuals involved.

Liposomes and emulsions are well known examples of delivery vehicles or carriers for hydrophobic drugs. Certain organic solvents such as dimethylsulfoxide also may be employed. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for stabilization may be employed.

Pharmaceutical compositions suitable for use in the present invention include compositions wherein the active ingredients are contained in an effective amount to achieve their intended purpose, to effect a therapeutic benefit, or to effect a detectable change in the function of a cell, tissue, or organ. More specifically, a therapeutically effective amount means an amount effective to prevent the development of or to alleviate the existing symptoms of the subject being treated. Determining the effective amount is well within the capability of those skilled in the art, especially in light of the detailed disclosure provided herein.

Toxicity and therapeutic efficacy of the compounds or compositions can be determined by standard pharmaceutical, pharmacological, and toxicological procedures in cell cultures or experimental animals. For example, numerous methods for determining the LD₅₀ (the dose lethal to 50% of the population) and the ED₅₀ (the dose therapeutically effective in 50% of the population) exist. The dose ratio between toxic and therapeutic effects is the therapeutic index, which can be expressed as the ratio between LD₅₀ and ED₅₀. Compounds and compositions exhibiting high therapeutic indices are preferred. The data obtained from cell culture assays or animal studies can be used in formulating a range of dosages for use in humans. [See, for example, Fingl et al., in The Phannacological Basis of Therapeutics, Ch. 1 p. 1 (1975)].

The compounds of the present invention may be administered by a single dose, multiple discrete doses or continuous infusion. Because the compounds preferably are non-peptidic, easily diffusible and relatively stable, they can be well-suited to continuous infusion.

Dose levels on the order of about 0.1 mg to about 10,000 mg of the active ingredient are useful in the treatment of the above conditions, with preferred levels being about 0.1 mg to about 1,000 mg. The specific dose level, and thus the therapeutically-effective amount, for any particular patient will vary depending upon a variety of factors, including the activity of the specific compound employed and its bioavailability at the site of drug action; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models also are helpful. The considerations for determining the proper dose levels are available to the skilled person.

Certain compounds can administered in lyophilized form. In this case, 1 to 1000 mg of a compound of the present invention may be lyophilized in individual vials, together with a carrier and a buffer, such as mannitol and sodium phospshate. The compound may be reconstituted in the vials with bacteriostatic water before administration.

In treating neurological disorders resulting from global or focal ischemia, for example, the compounds of the present invention are preferably administered orally, rectally, parenterally or topically at least 1 to 6 times daily, and may follow an initial bolus dose of higher concentration.

For the compounds, methods, and uses of the present invention, any administration regimen regulating the timing and sequence of drug delivery can be used and repeated as necessary to effect treatment. Such regimen may include pretreatment and/or co-administration with additional therapeutic agents.

### ILLUSTRATIVE EXAMPLES

### Synthetic Routes to Production of Exemplary Compounds of the Invention

### Example 1

Compounds containing N-sulfonyl linkages may be prepared according to the general method of Scheme I.

Preparation of [(3,5-dichlorophenyl)amino]-N-(3-{[(4-methoxylihenyl)sulfonyl][(4-methylphenyl)sulfonyl]amino}phenyl)formamide (Compound 1):

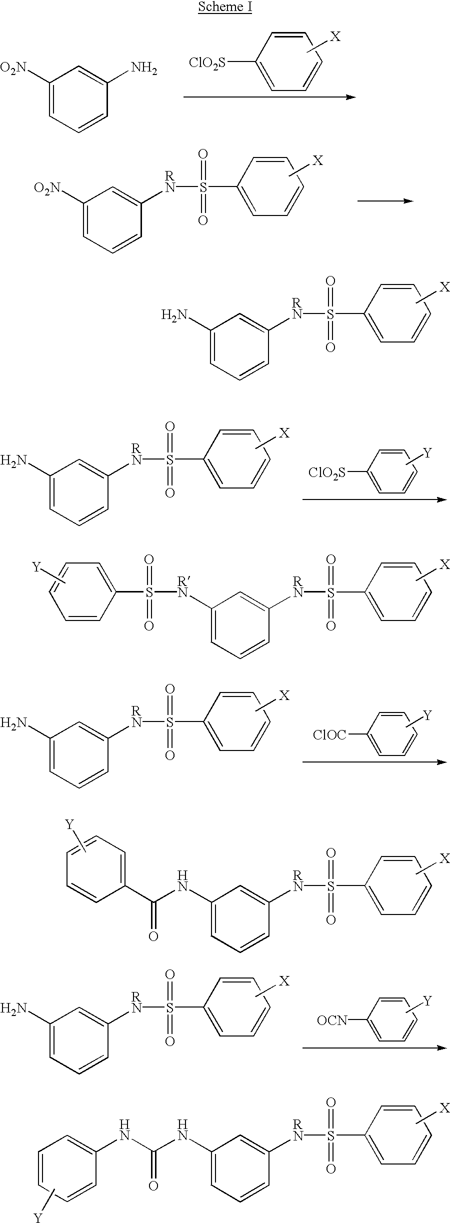

N-(3-{[(4-methoxyphenyl)sulfonyl][4-methylphenyl)sulfonyl]-3-nitroaniline. A solution of 3-nitroaniline (2 mmol), triethylamine (2 mmol), 4-methyphenyl sulfonyl chloride (2 mmol), and 4-methoxyphenylsulfonyl chloride (2 mmol) in dimethylacetamide (5 ml) was stirred at room temperature overnight. The reaction mixture was poured into ice-water, filtered, and the solid collected was recrystallized to obtain the title compound.

N-(3-{[(4-methoxyphenyl)sulfonyl][4-methylphenyl)sulfonyl]-1,3-diaminobenze and conversion to [(3,5-dichlorophenyl)amino]-N-(3-{[(4-methoxyphenyl) sulfonyl][(4-methylphenyl)sulfonyl]amino}phenyl)formamide. A mixture of the nitro compound (700 mg) and indium metal (3 g) in 15 mL of ethanol+4.5 mL of saturated ammonium chloride was refluxed for 4 hours. The reaction mixture was filtered through Celite and concentrated, and the crude amine was dissolved in 10 mL of dimethylacetamide and treated with triethylamine (2 mmol) and 3,5-dichlorophenylisocyanate (0.5 mmol). The reaction mixture was stirred at room temperature overnight and then poured into ice water. The crude solid collected upon filtration was dissolved in acetonitrile and chromatographed by HPLC, using a gradient elution from 5% water/95% acetonitrile with 0.1% TFA, to 100% acetonitrile with 0.1% TFA, to obtain Compound 1 as a white solid, ¹H NMR (DMSO-d₆, 400 MHz) δ 2.46(s, 3H); 3.90(s, 3H); 6.57(d, 1H);

7.20(m, 3H); 7.32(m, 2H); 7.5(d, 3H); 7.53(d, 2H); 7.73(m, 4H); 9.04(s, 1H); 9.11(s, 1H). Anal: Calcd for: C, 52.26; H, 3.74; N, 6.77; S, 10.33. Found: C, 52.01; H, 3.83; N, 6.78; S, 10.31.

### Example 2

Preparation of (3,5-Dichlorophenyl)-N-(3-{[(4-methoxyphenyl)sulfonyl][(4-methylphenyl)sulfonyl]amino}phenyl)formamide (Compound 3).

A mixture of N-(3-{[(4-methoxyphenyl)sulfonyl][4-methylphenyl)sulfonyl]-1,3-diaminobenze, prepared as described above, 170 mg, in 4 mL of dimethylacetamide, was treated with triethylamine (1 mmol) and 3,5-dichloro-benzoyl chloride (0.5 mmol). The reaction mixture was stirred at room temperature overnight and then poured into ice water. The crude solid collected upon filtration was dissolved in acetonitrile and chromatographed by HPLC, using a gradient elution from 5% water/95% acetonitrile with 0.1% TFA, to 100% acetonitrile with 0.1% TFA, to obtain compound 3 as a yellow solid, ¹H NMR (DMSO-d₆, 400 MHz) δ 2.48(s, 3H); 3.92(s, 3H); 6.66(d, 1H); 7.18(d, 2H); 7.34(t, 1H); 7.48(d, 2H); 7.67(t, 1H); 7.8(m, 5H); 8.05(m, 3H); 10.53(s, 1H). Anal: Calcd for: C, 53.56; H, 3.66; N, 4.63; S, 10.59. Found: C, 53.71; H, 3.84; N, 4.60; S, 10.40.

### Example 3

Preparation of (3-{bis[(3,5-dichlorophenyl)sulfonyl]amino}phenyl)[(4-methoxy-phenyl)sulfonyl][(4-methylphenyl)sulfonyl]amine (Compound 4). A mixture of N-(3-{[(4-methoxyphenyl)sulfonyl][4-methylphenyl)sulfonyl]-1,3-diaminobenze, prepared as described above, 170 mg, in 4 mL of dimethylacetamide, was treated with triethylamine (1 mmol) and 3,5-dichlorosulfonyl chloride (0.5 mmol).). The reaction mixture was stirred at room temperature overnight and then poured into ice water. The crude solid collected upon filtration was dissolved in acetonitrile and chromatographed by HPLC, using a gradient elution from 5% water/95% acetonitrile with 0.1% TFA, to 100% acetonitrile with 0.1% TFA, to obtain Compound 4 as a white solid, ¹H NMR (DMSO-d₆, 400 MHz) δ 2.45(s, 3H); 3.91(s, 3H); 6.8(t, 1H); 7.15(d, 2H); 7.26(d, 1H); 7.4(d, 1H); 7.43(d, 2H); 7.58(t, 1H); 7.75(q, 4H); 7.85(d, 4H); 8.11(t, 2H). Anal.: Calc'd for: C, 45.42; H, 3; N, 3.21; S, 14.70. Found: C, 45.91; H, 3.18; N, 3.28; S, 14.70. Compounds 2 and 5-10 were prepared in the same manner:

### Example 4

[(3,5-dichlorophenyl)amino]-N-(3-{bis[(4-methylphenyl)sulfonyl]amino}phenyl)formamide (Compound 2). ¹H NMR (DMSO-d₆, 400 MHz) δ 2.46(s, 6H); 6.57(d, 1H); 7.19(t, 1H); 7.33(m, 2H); 7.48(d, 5H); 7.54(d, 2H); 7.70(d, 4H); 9.05(s, 1H); 9.12(s, 1H). Anal: Calcd for: C, 53.64; H, 3.83; N, 6.95; S, 10.61. Found: C, 53.70; H, 4.04; N, 6.97; S, 10.43.

### Example 5

bis[(3,5-dichlorophenyl)sulfonyl](3-{[(naphthylamino) thioxomethyl]amino}phenyl)amine (Compound 5). ¹H NMR (DMSO-d₆, 400 MHz) δ 6.85(d, 1H); 7.41(t, 1H); 7.53(m, 3H); 7.63(d, 2H); 7.85(s, 1H); 7.87(d, 4H) 7.95(d, 1H); 8.01(s, 1H); 8.08(d, 1H); 8.13(t, 2H); 9.97(s, 1H); 9.99(s, 1H). Anal.: Calc'd. for: C, 48.96; H, 2.69; N, 5.91; S, 13.52; Cl, 19.93. Found: C, 49.29; H, 2.86; N, 5.92; S, 13.31; Cl, 19.75.

### Example 6

N-(3-{bis[(3,5-dichlorophenyl)sulfonyl]amino}phenyl)[(2,6-dichlorophenyl)amino]formamide (Compound 6). ¹H NMR (DMSO-d₆, 400 MHz) δ 6.68(d, 1H); 7.34(m, 2H); 7.49(m, 3H); 7.61(d, 1H); 7.84(d, 4H); 8.13(t, 2H); 8.24(s, 1H); 9.26(s, 1H). Anal.: Calc'd for: C, 42.90; H, 2.60; N, 5.48; S, 8.36. Found: C, 43.05; H, 2.47; N, 5.96; S, 8.54.

### Example 7

N-(3-{bis[(3,5-dichlorophenyl)sulfonyl]amino}phenyl)[(3,5-dichlorophenyl)amino]formamide (Compound 7). ¹H NMR (DMSO-d₆, 400 MHz) δ 6.84(t, 1H); 7.19(t, 1H); 7.43(d, 2H); 7.54(s, 1H); 7.60(d, 2H); 7.84(d, 4H); 8.22(t, 2H); 9.14(s, 1H); 9.21(s, 1H). Anal.: Calc'd for: C, 43.41; H, 2.89; N, 5.24; S, 7.99. Found: C, 43.60; H, 2.94; N, 5.44; S, 8.07.

### Example 8

(3,5-Dichlorophenyl)-N-{3-[bis(2-naphthylsulfonyl)amino]phenyl}formamide (Compound 8). ¹H NMR (DMSO-d₆, 400 MHz) δ 6.77(d, 1H); 7.40(t, 1H); 7.73(m, 3H); 7.81(t, 2H); 7.91(m, 6H); 8.15(t, 4H); 8.25(d, 2H); 8.50(s, 2H); 10.56(s, 1H). Anal: Calcd for: C, 59.58; H, 3.71; N, 3.97; S, 9.09; Cl, 10.05. Found: C, 59.29; H, 3.69; N, 4.07; S, 9.26; Cl, 10.41.

### Example 9

N-(3-{bis[(3,5-dichlorophenyl)sulfonyl]amino}phenyl)(3,5-dichlorophenyl)formamide (Compound 8a). ¹H NMR (DMSO-d₆, 400 MHz) δ 6.98(d, 1H); 15 7.53(t, 1H); 7.70(s, 1H); 7.82(s, 4H); 7.92(s, 1H); 7.96(s, 2H); 8.25(s, 2H); 10.64(s, 1H). Anal: Calcd for: C, 43.30; H, 2.24; N, 3.88; S, 8.89; Cl, 29.49. Found: C, 43.35; H, 2.36; N, 3.96; S, 8.92; Cl, 28.34.

### Example 10

(3-{Bis[(3,5-dichlorophenyl)sulfonyl]amino}phenyl)bis(2-naphthylsulfonyl)amine (Compound 9). ¹H NMR (DMSO-d₆, 400 MHz) δ 7.03(t, 1H); 7.34(d, 1H); 7.45(d, 1H); 7.59(t, 1H); 7.69(t, 2H); 7.78(t, 2H); 7.83(d, 4H); 7.88(d, 2H);

8.12(m, 6H); 8.20(d, 2H); 8.51(s, 2H). Anal: Calcd for: C, 50.34; H, 2.67; N, 3.09; S, 14.15. Found: C, 50.45; H, 2.87; N, 3.17; S, 14.12.

### Example 11

(3-{Bis[(3,5-dichlorolphenyl)sulfonyl]amino}phenyl)bis[(4-methoxyphenyl)sulfonyl]amine (Compound 10). ¹H NMR (DMSO-d₆, 400 MHz) δ 3.89(s, 6H); 6.65(t, 1H); 7.17(d, 4H); 7.27(d, 1H); 7.50(d, 1H); 7.60(t, 1H); 7.68(d, 4H); 7.78(d, 4H); 8.23(t, 2H). Anal: Calcd for: C, 44.35; H, 2.79; N, 3.23; S, 14.80. Found: C, 44.63; H, 2.96; N, 3.36; S, 14.66.

Compounds 11-16, and 27-29 were prepared according to the following Scheme II.

### Example 12

Preparation of {[3,5-bis(trifluoromethyl)phenyl]amino}({2-[({[3,5-bis(trifluoromethyl)phenyl]amino}thioxomethyl)amino]cyclohexyl}amino)methane-1-thione (Compound 12).

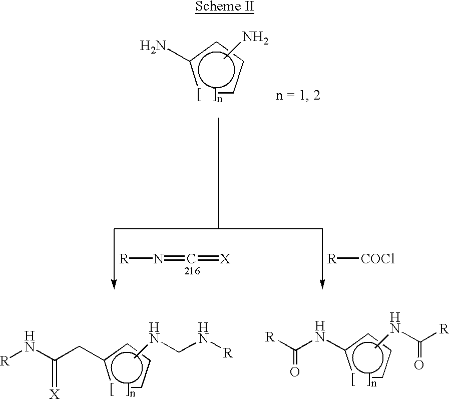

A mixture of trans-1,2-diaminocyclohexane (0.25 g; 2.2 mmol) and 3,5-trifluoro-methyl-phenylisothiocyanate (1.25 g; 4.6 mmol) in methylene chloride was stirred overnight. The formed precipitate was collected by filtration, washed with methylene chloride, and dried under vacuum to provide Compound 12 as an analytically pure white solid, ¹H NMR (DMSO-d₆, 400 MHz) δ 1.42-1.84(m, 8H); 4.65-4.75(m, 2H); 7.73(s, 2H); 7.93-8.07(m, 2H); 8.30(s, 4H); 10.14(s, 2H). Anal: Calcd for: C, 43.91; H, 3.07; N, 8.53; S, 9.77. Found: C, 44.01; H, 3.21; N, 8.49; S, 10.

### Example 13

(Naphthylamino)[(2-{[(naphthylamino)thioxomethyl]amino}cyclohexyl)amino]methane-1-thione (Compound 11). ¹H NMR (DMSO-d₆, 400 MHz) δ 1.40-1.82(m, 8H); 4.69-4.79(m, 2H); 7.41-7.57(m, 8H); 7.59-7.69(m, 2H); 7.80(d, 2H, J=8.2); 7.89-7.99(m, 4H); 9.67(s, 2H). Anal: Calcd for: C, 68.15; H, 6.10; N, 10.60; S, 12.13. Found: C, 68.46; H, 6.44; N, 10.21; S, 11.94.

### Example 14

[(4-iodophenyl)amino]{[2-({[(4-iodophenyl)amino]thioxomethyl}amino)cyclohexyl]amino}methane-1-thione (Compound 13). ¹H NMR (DMSO-d₆, 400 MHz) δ 1.38-1.80(m, 8H); 4.59-4.69(m, 2H); 7.38(d, 4H, J=8.4); 7.61(d, 4H, J=8.6); 7.65(d, 2H, J=8.2); 9.69(s, 2H). Anal: Calcd for: C, 37.75; H, 3.48; N, 8.80; S, 10.08; I, 39.88. Found: C, 37.55; H, 3.53; N, 8.65; S, 10.13; I, 39.97.

### Example 15

[(3,4-dichlorophenyl)amino]{[2-({[(3,4-dichlorophenyl)amino]thioxomethyl}amino)cyclohexyl]amino}methane-1-thione (Compound 14). ¹H NMR (DMSO-d₆, 400 MHz) δ 1.38-1.80(m, 8H); 4.60-4.70(m, 2H); 7.40(dd, 2H, J=2.4, 8.8); 7.52(d, 2H, J=8.8); 7.79(d, 2H, J=8.4); 8.09(s, 2H); 9.82(s, 2H). Anal: Calcd for: C, 45.99; H, 3.86; N, 10.73; S, 12.28. Found: C, 46.09; H, 3.93; N, 10.64; S, 12.39.

### Example 16

[(3,5-dichlorophenyl)amino]{[2-({[(3,5-dichlorophenyl)amino]thioxomethyl}amino)cyclohexyl]amino}methane-1-thione (Compound 15). ¹H NMR (DMSO-d₆, 400 MHz) δ 1.46(m, 2H); 1.56(m, 2H); 1.69(m, 4H); 4.66(m, 2H); 7.26(t, 2H, J=2); 7.67(s, 4H); 7.88(d, 2H, J=8); 9.87(s, 2H). Anal: Calcd for: C, 45.99; H, 3.86; N, 10.73; S, 12.28. Found: C, 46.05; H, 3.91; N, 10.83; S, 12.19.

### Example 17

cis-[(3,5-dichlorophenyl)amino]-N-(2-{[(3,5-dichlorophenyl)amino]carbonylamino}cyclohexyl)formamide (Compound 15a). ¹H NMR (DMSO-d₆, 400 MHz) δ 1.41.46(m, 6H); 1.62(m, 2H); 3.85(m, 2H); 6.33(d, 2H, J=8); 7.07(t, 2H, J=2); 7.43(d, 4H, J=2); 8.83(s, 2H). Anal: Calcd for: C, 48.65; H, 4.16; N, 11.35; Cl, 28.72. Found: C, 48.49; H, 3.89; N, 11.13; Cl, 28.58.

### Example 18

cis-[(3,5-Dichlorophenyl)amino]-N-(4-{[(3,5-dichlorolphenyl)amino]carbonylamino}cyclohexyl)formamide (Compound 16). ¹H NMR (DMSO-d₆, 400 MHz) δ 1.27(t, 4H, J=9); 1.87(d, 4H, J=6); 3.33(m, 2H); 6.30(d, 2H, J=8); 7.06(s, 2H); 7.45 (s, 4H); 8.72(s, 2H). Anal: Calcd for: C, 49; H, 4.11; N, 11.43; Cl, 28.93. Found: C, 48.60; H, 4.22; N, 11.33; Cl, 28.66.

### Example 19

[(3,5-dichlorophenyl)amino]-N-(2-{[(3,5-dichlorophenyl)amino]carbonylamino}phenyl)formamide (Compound 27). ¹H NMR (DMSO-d₆, 400 MHz) δ 7.16(s, 4H); 7.54(s, 6H); 8.24(s, 2H); 9.52(s, 2H). Anal: Calcd for: C, 49.61; H, 2.91; N, 11.57. Found: C, 49.37; H, 3.01; N, 11.41.

### Example 20

[(3,5-dichlorophenyl)amino]{[2-({[(3,5-dichlorophenyl)amino]thioxomethyl}amino)phenyl]amino}methane-1-thione (Compound 28). ¹H NMR (DMSO-d₆, 400 MHz) δ 7.31(m, 6H); 7.45(m,3H); 7.59(s, 5H). Anal: Calcd for: C, 46.53; H, 2.73; N, 10.85; S, 12.42. Found: C, 46.69; H, 2.82; N, 10.86; S, 12.46.

### Example 21

(4-Iodophenyl)-N-{2-[(4-iodophenyl)carbonylamino]phenyl}formamide (Compound 29). ¹H NMR (DMSO-d₆, 400 MHz) δ 7.28-7.30 (m, 2H); 7.62-7.65 (m, 2H); 7.71 (d, 4H, J=8.4); 7.91 (d, 4H, J=8.4); 10.05 (s, 2H). Anal: Calcd for: C, 42.28; H, 2.48; N, 4.93; I, 44.67. Found: C, 42.43; H, 2.52; N, 4.85; I, 44.70.

### Example 22

Compound 26 was prepared in a similar manner, by the reaction of 3-phenoxyaniline with 3,5-trifluoromethylphenylisocyanate: {[3,5-bis(trifluoromethyl)phenyl]amino}-N-(3-phenoxyphenyl)formamide (Compound 26). A mixture of 3-phenoxyaniline (1 mmol) and 3,5-trifluoromethyl-phenylisocyanate (1 mmol) in methylene chloride (8 mL) was stirred overnight. The reaction mixture was filtered to remove solids, and evaporation of the solvent provided the desired compound in pure form as a white solid, ¹H NMR (Acetone-d₆, 400 MHz) δ 6.69 (dt, 1H, J=2.0,7.0 Hz); 7.06 (d, 2H, J=8.5 Hz); 7.16 (t, 1H, J=7.5 Hz); 7.28-7.34 (m, 3H); 7.41 (t, 2H, J=7.5 Hz); 7.62(s, 1H); 8.19 (s, 2H); 8.48 (br s, 1H); 8.74 (br s, 1H). Anal: Calcd for: C, 57.28; H, 3.20; N, 6.36. Found: C, 57.34; H, 3.07; N, 6.43.

The synthesis of Compounds 17-19 was conducted according to the following Scheme III.

### Example 23

Synthesis of (1R,3S)-N-(3,5-dichlorophenyl)[3-({[(3,5-dichlorophenyl)amino]thioxomethyl}amino)cyclopentyl]formamide (Compound 17).

4-tert-Butoxycarbonylamino-cyclopentanecarboxylic acid. 4-Amino-cyclopent-2-enecarboxylic acid (1.02 g; 8 mmol), tert-butoxycarbonyl anhydride (8 mmol), and triethylamine (8 mmol) were stirred together overnight in 25 mL of tetrahydrofuran. The reaction mixture was poured into water, the pH was adjusted to 3.5, and the product was extracted into ethyl acetate. The ethyl acetate phase was dried and concentrated, and the crude product was dissolved in methylene chloride (25 mL), treated with a catalytic amount of 10% palladium on carbon, and hydrogenated overnight at atmospheric pressure. The reaction mixture was filtered through Celite and concentrated to provide the desired product.

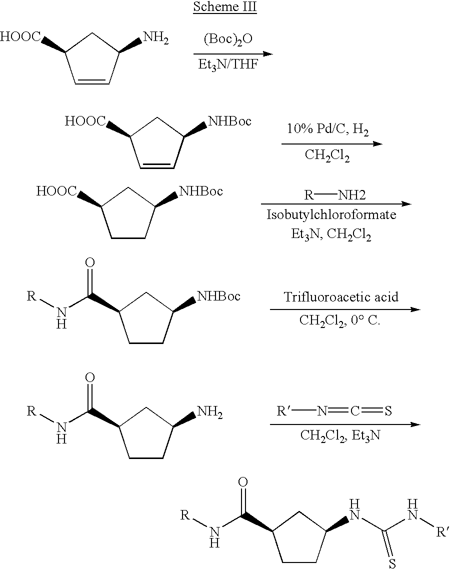

[3-(3,5-Dichlorophenylcarbamoyl)cyclopentyl]carbamic acid tert-butyl ester. A solution of 4-tert-Butoxycarbonylamino-cyclopentanecarboxylic acid (1 mmol) and triethylamine (5 mmol) in 10 mL of methylene chloride was treated with isobutylchloroformate (1.2 mmol) and stirred for 5 min at 0° C. A solution of 3,5-dichloroaniline (1 mmol) in 5 mL of methlene chloride was added, and the resulting mixture was stirred at 0° C. for 2 hours. It was concentrated and the crude residue was purified on a silica gel column, eluting with 25% ethyl acetate in hexane, to obtain the product, ¹H NMR (CDCl3, 400 MHz) δ 7.54 (m, 3H); 5.34 (m, 1H); 4.05 (m, 1H); 2.98 (m, 1H); 2.15 (m, 1H); 1.75-1.86 (m, 4H); 1.52 (s, 9H).

(1R,3S)-N-(3,5-dichlorophenyl)[3-({[(3,5-dichlorophenyl)amino]thioxomethyl}amino)cvclopentyl]formamide (Compound 17). [3-(3,5-Dichloro-phenylcarbamoyl)cyclopentyl]carbamic acid tert-butyl ester (0.5 mmol) in 5 mL of methylene chloride was cooled to OoC and treated with 2.5 mL of trifluoroacetic acid. After stirring for two hours the solvent was removed in vacuo, and the residue was taken up in 10 mL of methylene chloride and treated with triethylamine (2 mmol) and 3,5-dichloroisothiocyanate (0.5 mmol). The mixture was stirred overnight at room temperature, concentrated in vacuo, and purified on a silica gel column to obtain Compound 17, ¹H NMR (CDCl3, 400 MHz) δ 8.21(s, NH, 1H), 7.77(s, NH, 1H), 7.53(s, NH, 1H), 7.35(s, 2H), 7.34(s, 1H), 7.15(s, 2H), 7.129s, 1H), 4.96(m, 1H), 2.90(m, 1H), 1.82-2.14(m, 6H). Anal: Calcd for: C, 47.32; H, 3.98; N, 8.69; S, 6.63; Cl, 30.06. Found: C, 47.06; H, 3.77; N, 8.26; S, 6.36; Cl, 30.01.

### Example 24

(1S,3R)-N-(3,5-dichlorophenyl)[4-({[(3,5-dichlorolphenyl)amino]thioxomethyl}amino)cyclopent-2-enyl]formamide (Compound 18). ¹H NMR (CDCl3, 400 MHz) δ 7.66(d, 1H, NH), 7.7.60(s, 1H, NH), 7.5(s, 1H, NH), 7.43(s, 2H), 7.33(s, 1H), 7.16(s, 2H), 7.139s, 1H), 6.14(m, 1H), 5.91(m, 1H), 5.30(t, 1H), 3.47(m, 1H), 2.38(m, 1H), 2.03(m, 1H) Anal: Calcd for: C, 48.18; H, 3.53; N, 8.87; S, 6.77; Cl, 29.94. Found: C, 48.33; H, 3.57; N, 8.40; S, 6.80; Cl, 29.33.

### Example 25

(1S,3R)-N-(3,5-dichlorophenyl)[3-({[(3,5-dichlorophenyl)amino]thioxomethyl}amino)cyclopentyl]formamide (Compound 19). ¹H NMR (CDCl3, 400 MHz) δ 8.21(d, NH, 1H), 7.77(s, 1H, NH), 7.38(s, 2H), 7.35(s, 1H), 7.35(s, 1H), 7.15(s, 2H), 7.13(s, 1H), 4.96(m, 1H), 2.90(m, 1H), 1.82-2.14(m, 6H). Anal.: Calcd for: C, 47.03; H, 3.97; N, 8.62; S, 6.57; Cl, 30.53. Found: C, 46.93; H, 3.75; N, 8.32; S, 6.41; Cl, 30.16.

### Example 26

The synthesis of N-(3,5-dichlorophenyl)-2-{3-[(3,5-dichlorolphenyl)carbonylamino]phenoxy}ethanamide (Compound 24) was conducted according to the following Scheme IV.

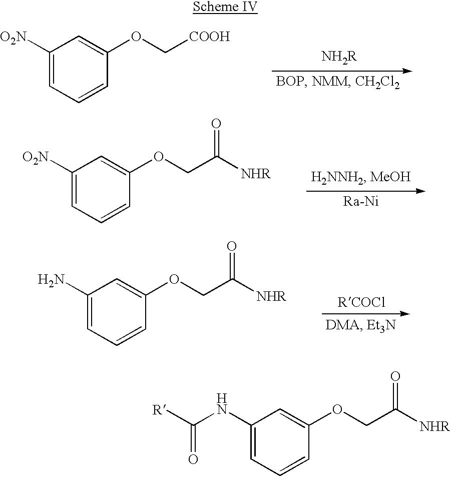

N-(3,5-Dichlorophenyl)-2-(3-nitrophenoxy)acetamide. A solution of (3-nitrophenoxy) acetic acid (900 mg), NMM (8.5 mmmol), BOP (5 mmol), and 3,5-dichloroaniline (5 mmol) in methylene chloride (40 mL) was stirred at room temperature overnight. The solvent was evaporated and the crude residue purified on a silica gel column (30% ethyl acetate/hexane) to obtain the product as a solid, M+=341 by mass spectrometry.

2-(3-Aminophenoxy)-N-(3,5-dichlorophenyl)acetamide. A mixture of hydrazine hydrate (20 mmol) and Raney-Nickel (catalytic) in 75 mL of methanol was heated to reflux and N-(3,5-Dichloro-phenyl)-2-(3-nitrophenoxy)acetamide (4 mmol) was added. The resulting mixture was refluxed for 30 min, cooled, filtered to remove solids, and concentrated. The product was carried directly into the next step.

N-(3,5-dichlorophenyl)-2-{3-[(3,5-dichlorophenyl)carbonylamino]phenoxy} ethanamide (Compound 24). A mixture of N-(3,5-dichlorophenyl)-2-{3-[(3,5-dichlorophenyl)carbonylamino]phenoxy} ethanamide (1 mmol) in dimethylacetamide (5 mL) was cooled to 0° C. and treated with triethylamine (0.5 mL) followed by 3,5-dichlorobenzoyl chloride (1 mmol). After stirring at 0° C. for 1 hour the mixture was poured into ice-water and allowed to stand overnight. The crystalized product was collected, washed with ether, and dried to obtain a yellow powder, ¹H NMR (DMSO-d₆+CD₂Cl₂, 400 MHz) δ 4.65(s, 2H); 6.78(d, 1H); 7.07(s, 1H); 7.28(t, 1H); 7.36(d, 1H); 7.65(s, 1H); 7.76(s, 2H); 7.84(s, 3H); 7.96(s, 2H). Anal: Calcd for: C, 52.10; H, 2.91; N, 5.79. Found: C, 52.37; H, 3.05; N, 5.82.

The synthesis of compounds 22 and 25 was conducted according to the following Scheme V.

### Example 27

Synthesis of 3-({[(3,5-dichlorophenyl)amino]thioxomethyl}amino)phenyl 2,3,4,5,6-pentafluorobenzenesulfonate (Compound 22).

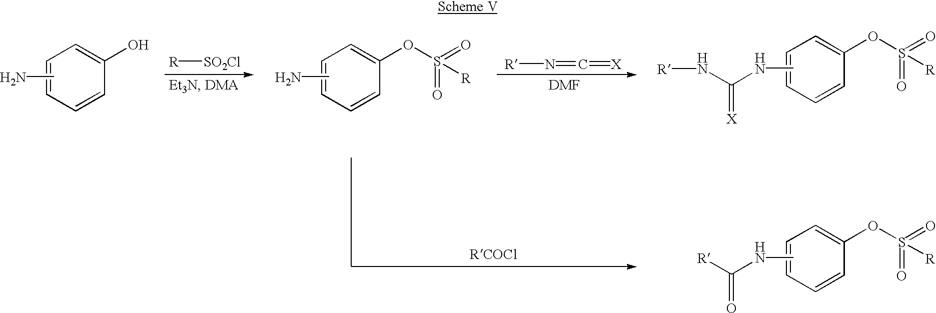

2,3,4,5,6-Pentafluorobenzenesulfonic acid 3-aminophenyl ester. Pentafluorobenzene-sulfonyl chloride (4 mmol) was added to a mixture of 3-aminophenol (4 mmol) and triethylamine (5 mmol) in dimethylacetamide (15 mL).

The mixture was stirred for four hours, concentrated, and purified on a silica gel column, eluting with chloroform to obtain the sulfonic ester as a white solid, ¹H NMR (DMSO-d₆, 400 MHz) δ 7.25 (t, 1H); 6.45 (d, 1H); 6.25 (s, 1H); 6.18 (d, 1H): 5.65 (br, 2H).

3-({[(3,5-dichlorophenyl)amino]thioxomethyl}amino)phenyl 2,3,4,5,6-pentafluorobenzenesulfonate (Compound 22). A mixture of 2,3,4,5,6-pentafluorobenzenesulfonic acid 3-aminophenyl ester (1.5 mmol) and 3,5-dichloroisothiocyanate (1.8 mmol) in dimethylformamide (15 mL) was stirred overnight. The mixture was poured into ice-water, and the solids which formed upon standing were collected by filtration and washed with ethyl acetate to obtain Compound 22 as a white solid, ¹H NMR (DMSO-d₆, 400 MHz) δ 7.11(d, 1H, J=7); 7.44(m, 6H); 10.15(s, 1H); 10.25(s, 1H). Anal: Calcd for: C, 42; H, 1.67; N, 5.16; S, 11.80; Cl, 13.05. Found: C, 42.08; H, 1.77; N, 5.22; S, 11.78; Cl, 13.10.

### Example 28

Synthesis of 3-[(3,5-dichlorophenyl)carbonylamino]phenyl 2,3,4,5,6-penta-fluorobenzenesulfonate (Compound 25). A mixture of 2,3,4,5,6-pentafluoroben-zenesulfonic acid 3-aminophenyl ester (0.74 mmol), 3,5-dichlorobenzoyl chloride (0.81 mmol), and triethylamine (0.88 mmol) in methylene chloride (10 mL) was stirred overnight. The formed precipitate was collected via filtration, washed with methylene chloride, and dried under vacuum to obtain Compound 25 as an analytically pure white solid, ¹H NMR (DMSO-d₆, 400 MHz) δ 7.03(dd, 1H, J=2,8); 7.47(t, 1H, J=8); 7.72(d, 1H, J=8); 7.76(t, 1H, J=2); 7.89(t, 1H, J=2); 7.95(d, 2H, J=2); 10.65(s, 1H). Anal: Calcd for: C, 44.55; H, 1.57; N, 2.73; S, 6.26; Cl, 13.84. Found: C, 44.34; H, 1.72; N, 2.78; S, 6.18; Cl, 13.99.

### Example 29

The synthesis of [(3,5-dichlorophenyl)amino]({3-[2,2-bis(4-chlorophenyl)vinyl]phenyl}amino methane-1-thione (Compound 20) was conducted according to the following Scheme VI.

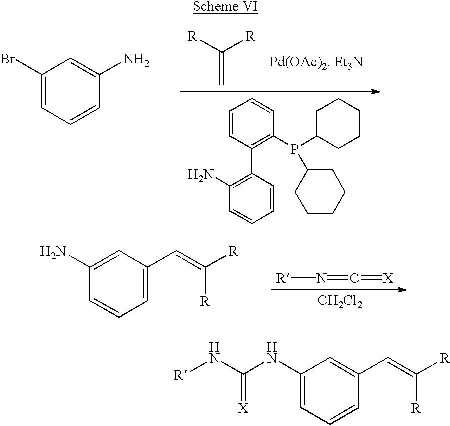

3-[2,2-Bis-(4-chlorophenyl)vinyl]phenylamine. A mixture of 3-bromoaniline (190 mg; 1.1 mmol), 1,1-(para-chloro)phenylethylene (250 mg; 1.0 mmol), palladium (II) acetate (22 mg; 0.1 mmol), and 2′-dicyclohexylphosphanyl-biphenyl-2-ylamine (122 mg; 0.5 mmol) in triethylamine (5 mL) was refluxed overnight. The solvent was evaporated and the crude residue was purified on a silica gel column, eluting with 50% ethyl acetate in hexane, to obtain the vinyl compound, ¹H NMR (DMSO-d₆, 400 MHz) d 7.34-7.14 (m, 8H); 6.98 (m, 1H); 6.87 (s, 1H); 6.48 (dd, 2H); 6.42 (s, 1H).

[(3,5-dichlorophenyl)amino]({3-[2,2-bis(4-chlorophenyl)vinyl]phenyl}amino methane-1-thione (Compound 20). A mixture of 3-[2,2-Bis-(4-chlorophenyl) vinyl]phenylamine (340 mg; 1.0 mmol) and 3,5-dichloroisothiocyanate (245 mg; 1.2 mmol) in methylene chloride (10 mL) was stirred overnight. The resulting precipitate was collected via filtration, washed with methylene chloride, and dried under vacuum to provide Compound 20 as a white solid, ¹H NMR (DMSO-d₆, 400 MHz) δ 6.78(d, 1H, J=7); 7.12(s, 1H); 7.14-7.20(m, 3H); 7.24(s, 1H); 7.30(t, 3H, J=8); 7.43(t, 4H, J=8); 7.55-7.58(m, 3H); 9.92(s, 1H); 10.01(s, 1H). Anal: Calcd for: C, 59.58; H, 3.33; N, 5.15; S, 5.89; Cl, 26.05. Found: C, 60.19; H, 3.95; N, 5.21; S, 5.74; Cl, 24.90.

### Example 30

The synthesis of ({3-[2-aza-2-(diphenylamino)vinyl]phenyl}amino)[(3,5-dichlorophenyl)amino]methane-1-thione (Compound 21) was conducted according to the following Scheme VII.

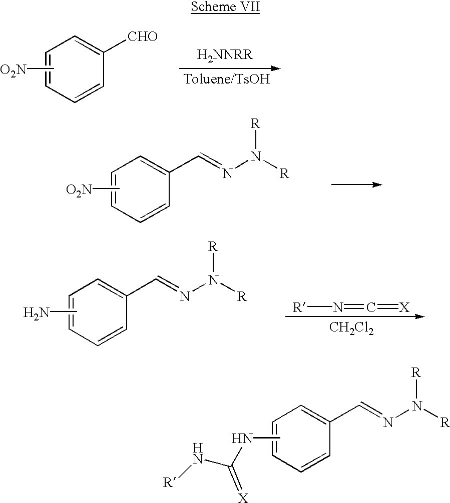

A mixture of 3-nitrobenzaldehyde (1 mmol), diphenylhydrazine (1 mmol), and p-toluenesulfonic acid (catalytic amount) in 20 mL of toluene was refluxed for 2 hours, using a Dean-Stark trap to remove the formed water. The solvent was removed under reduced pressure, and the crude material was taken up in 10 mL of methylene chloride, treated with a catalytic amount of 10% Pd/C, and hydrogenated at atmospheric pressure for 20 minutes. The mixture was filtered through Celite to remove the catalyst, and 3,5-dichloroisothiocyanate was added and the resulting mixture was stirred overnight at room temperature. Removal of the solvent and purification of the crude product on a silica gel column delivered Compound 21 as a white solid, ¹H NMR (CDCl₃, 400 MHz) δ 8.24(br, NH, 1H), 7.759br, NH, 1H), 7.59(s, 1H), 7.49(d, 1H), 7.36-7.44(m, 11H), 7.24(d, 1H), 7.19(s, 4H), 7.16(s, 2H), 7.08(s, 1H) Anal: Calcd for: C, 60.99; H, 4.21; N, 10.94; S, 6.26. Found: C, 60.99; H, 4.14; N, 10.65; S, 6.13.

### Example 31

The synthesis of 1-{3-[3,5-Bis(trifluoromethyl) benzyloxyl]phenyl}-5-(3,5-dichlorophenyl)-1,4-dioxo-2,3,5-triazapentane (Compound 23) was conducted according to the following Scheme VIII.

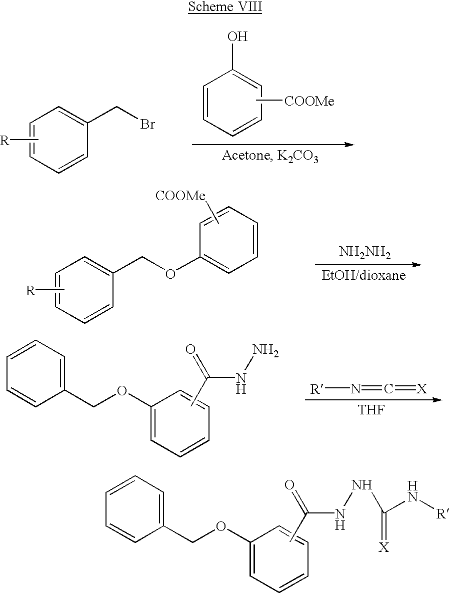

3-(3,5-Bis-trifluoromethylbenzyloxy)benzoic acid hydrazide. A mixture of 3,5-bis-trifluoromethylbenzyl bromide (2.3 g; 7.5 mmol), 3-hydroxybenzoic acid methyl ester (1.26 g; 8.2 mmol), and potassium carbonate (2.07g; 15 mmol) in 150 mL of acetone was stirred and treated with 18-crown-6 crown ether (80 mg). The resulting mixture was refluxed overnight, then cooled to room temperature and concentrated. The crude oily product was suspended in a mixture of ethanol (50 mL) and dioxane (75 mL), and hydrazine monohydrate (1.13 g; 22.5 mmol) was added. The mixture was stirred and refluxed overnight, cooled, and poured into 200 ml of water. After stirring for 5 min, a white precipitate formed; 30 mL of 1N NaOH was added and stirring was continued for another 5 min. The solids were collected, washed with water, and air-dried to obtain 1.88 g of the hydrazide.

1-{3-[3,5-Bis(trifluoromethyl)benzyloxyl]phenyl}-5-(3,5-dichlorophenyl)-1,4-dioxo-2,3,5-triazapentane (Compound 23). To a stirred solution of 3-(3,5-bis-trifluoromethylbenzyloxy)benzoic acid hydrazide (189 mg; 0.5 mmol) in tetrahydrofuran (30 mL) was added 3,5-dichloroisothiocyanate (102 mg; 0.5 mmol), and the resulting mixture was stirred and refluxed for 1 hour, then stirred at room temperature for 20 h. The solvent was removed in vacuo, and the solid product was triturated with ether/hexanes and air-dried to deliver 100 mg of Compound 23 as a white solid.

¹H NMR (DMSO-d₆, 400 MHz) (All signals have distinct satellites-amido-imidol tautomerism—thus, only major ones are listed) δ 10.94 (s, 1H); 10.54 (s+s, 2H); 8.22 (s, 2H); 8.12 (s, 1H); 7.75-7.30 (m, 3H); 7.60-7.50 (m, 2H); 7.41-7.34 (m, 2H); 5.42 (s, 2H). Anal: Calcd for: C, 47.72; H, 2.86. Found: C, 47.52; H, 2.51.

Compounds 30 and 31 were prepared according to the following Scheme IX.

### Example 32

1-{3-[(3-Benzyloxy)phenylcarboxamido]benzoyl}-2-(3,5-dichlorobenzoyl)hydrazine (Compound 30). ¹H NMR (DMSO-d₆, 400 MHz) δ 10.78 (s, 1H); 10.65 (s, 1H); 10.43 (s, 1H); 8.31 (s, 1H); 8.03 (d, J=8.0 Hz, 1H); 7.95 (s, 2H); 7.92 (s, 1H); 7.70-7.30 (m, 10H); 7.26 (d, J=8.5 Hz, 1H); 5.21 (s, 2H).

Anal.: Calcd. for: C, 60.88; H, 4.20; N, 7.61. Found: C, 60.52; H, 4.25; N, 7.33. Physical form: white solid.

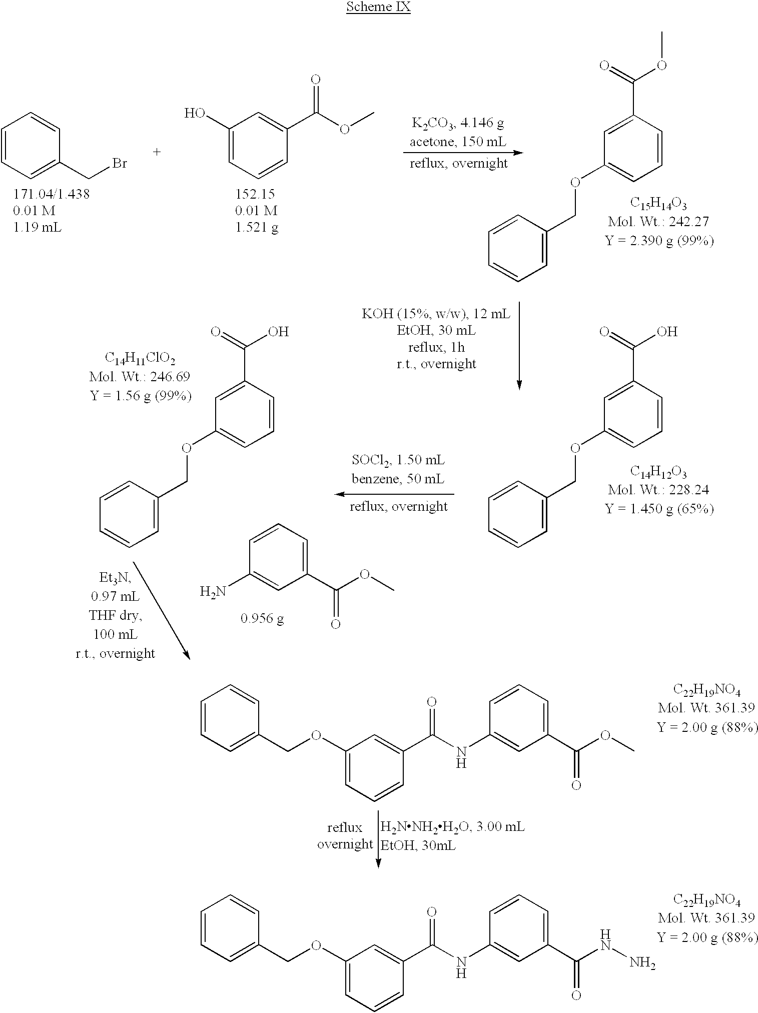

### Example 33

1-{3-[(3-Benzyloxy)phenylcarboxamido]benzoyl}-2-(3,4-dichlorobenzenesulfonyl)hydrazine (Compound 31).). ¹H NMR (DMSO-d₆, 400 MHz) δ 11.69 (s, 1H); 10.79 (s, 1H); 10.42 (s, 1H); 8.36-7.92 (m, 4H); 7.83 (d, J=8.5 Hz, 1H); 7.69-7.20 (m, 11H); 5.20 (s, 2H). Anal.: Calcd. for: C, 53.47; H, 4.15; N, 6.93. Found: C, 53.64; H, 3.65; N, 6.39. Physical form: white solid.

Compounds 32, 33, 37, 38, and 41 were prepared according to the following Schemes X and XI:

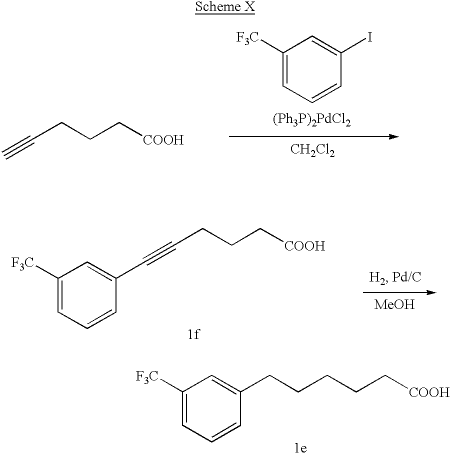

6-(3-Trifluoromethylphenyl)hex-5-ynoic acid if (Scheme X). To a solution of hexynoic acid (1.25 g, 11.1 mmol) and 3-iodobenzotrifluoride (3.33 g, 12.2 mmol) in 50 mL dichloromethane was added dichlorobis (triphenyiphoisphine) palladium(II) (0.39 g, 0.56 mmol), copper(I) iodide (0.11 g, 0.56 mmol) and triethylamine (2.25 g, 22.2 mmol) and the mixture refluxed 2d. The mixture was then concentrated and the product purified on silica with 2:1 hexane:EtOAc to give 1f as a yellow oil, 1.65 g (58%): ¹H NMR (CDCl3, 400 MHz): δ 1.74-1.83(m, 2H); 2.40(t, J=7.3 Hz, 2H); 2.50(t, J=7.1 Hz, 2H); 7.60(t, J=7.8 Hz, 1H); 7.68-7.75(m, 3H); 12.17(s, 1H). TLC: R_{f}=0.3 (1:2 EtOAc:Hexane).

6-(3-Trifluoromethylphenyl)hexanoic acid (1e, Scheme X). A solution of 1f (1.5 g, 5.8 mmol) in 15 mL methanol containing 0.25 g 10% palladium on carbon was hydrogenated at 50 psi hydrogen for 3 h. The mixture was then filtered through celite and concentrated to give 1e as a clear oil, 1.45 g (95%): ¹H NMR (CDCl3, 400 MHz): δ 1.22-1.36(m, 2H); 1.48-1.66(m, 4H); 2.20(t, J=7.3 Hz, 2H); 2.67(t, J=7.8 Hz, 2H); 7.49-7.57(m, 4H); 12.04(bs, 1H).

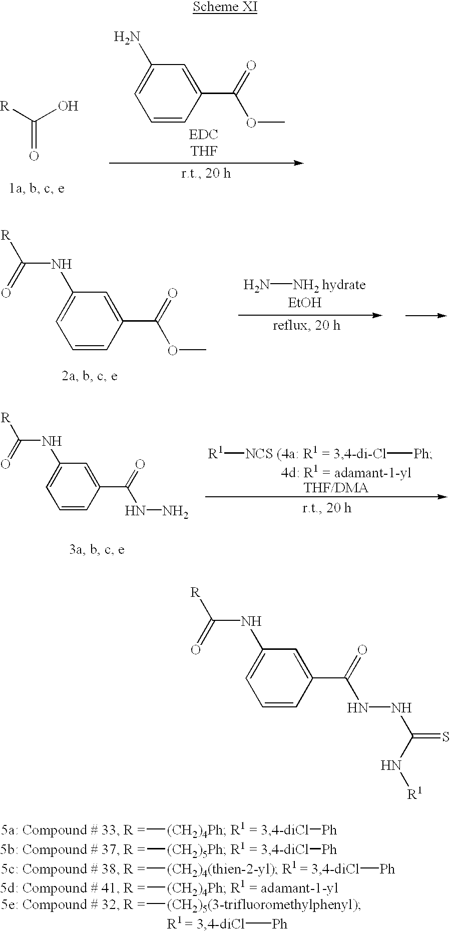

3-(Acylamino)benzoic acid methyl ester (2a-c,e, Scheme XI). A mixture of the corresponding acid 1a-c,e (5 mmol), methyl 3-aminobenzoate (0.72 g, 5 mmol), and 1-[3-(dimethylamino)propyl]-3-ethylcarbodiimide hyfrochloride (EDC, 1.08 g, 5.7 mmol) in dry THF (30 mL) was stirred at room temperature for 20 h. The whole was poured onto ice-water mixture (100 g) and left stand aside for 2 h. The product formed was extracted with methylene chloride (75 mL). Organic layer separated, dried (Na₂SO₄ ahyd.), and solvents evaporated in vacuum. Depending on the appearance of the product, it was used as is (solid) or further subjected to column chromatography (oil), using silica gel and EtOAc:hexanes 1:2 eluting system. Yields of the products vary from 58% to 75%.

3-(Acylamino)benzoic acid hydrazide (3a-c,e, Scheme XI). A solution of the corresponding ester 2a,b,c,e (5 mmol) and hydrazine hydrate (8 mL) in the mixture of propanol-2 or ethanol (100 mL) and water (1 mL) was stirred and refluxed for 20 h. Solvent evaporated in vacuum to produce yellowish-white solid, which was triturated with a minimum amount of ethanol (5-8 mL), filtered, and air-dried. Yields of the products vary from 48 to 65%.

### Example 34

1-{3-[(5-Phenyl)valeroylamino]benzoyl}-4-(3,4-dichlorophenyl)thiosemicarbazide (Compound 33; 5a in Scheme XI). To a stirred solution of hydrazide 3a (0.093 g, 0.3 mmol) in the mixture of THF (4 mL) and DMA (1 mL) was added a solution of isothiocyanate 4a (0.043 mL, 0.3 mmol) in THF (1 mL) in one portion. The whole was stirred for 18 h, poured onto ice-water. The residue formed was filtered and then recrystallized from aq. EtOH to give Compound 33. Yield 0.099 g (64%). Off-white soild; ¹H NMR (DMSO-d₆, 400 MHz), δ 10.56 (s, 1H); 10.08 (s, 1H); 9.99 (s, 1H); 9.90 (br.s, 1H); 8.10 (s, 1H); 7.84 (d, J=8.0 Hz, 1H); 7.65-7.50 (M, 3H); 7.41 (t, J=7.8 Hz); 7.30-7.11 (m, 5H); 2.64-2.57 (m, 2H); 2.38-2.31 (m, 2H); 1.65-1.58 (m, 4H). Anal: Calcd. for C₂₅H₂₄Cl₂N₄O₂S: C, 58.25; H, 4,69; N, 10.87. Found: C, 58.06; H, 4.75; 64. N, 10.97.

### Example 35

1-{3-[(7-Phenyl)heptanoylamino]benzoyl}-4-(3,4-dichlorophenyl)thiosemicarbazide (Compound 37; 5b in Scheme XI). To a stirred solution of hydrazide 3b (0.203 g, 0.6 mmol) in DMA (15 mL) was added isothiocyanate 4a (0.086 mL, 0.6 mmol) in one portion. The whole was stirred for 96 h, poured onto ice-water. The residue formed was filtered, washed with water, and air-dried to give Compound 37. Yield 0.251 g (77%). White solid; ¹H NMR (DMSO-d₆, 400 MHz), δ 10.56 (s, 1H); 10.06 (s, 1H); 9.99 (s, 1H); 9.91 (br.s, 1H); 8.10 (s, 1H); 7.84 (d, J=7.8 Hz, 1H); 7.67-7.48 (m, 3H); 7.41 (t, J=7.8 Hz, 1H); 7.29-7.09 (m, 5H); 2.56 (t, J=7.3 Hz, 2H); 2.31 (t, J=7.3 Hz, 2H); 1.64-1.51 (m, 4H); 1.44-1.25 (m, 4H). Anal: Calcd. for C₂₇H₂₈Cl₂N₄O₂S: C, 59.67; H, 5.19; N, 10.31. Found: C, 59.84; H, 5.10; 64. N, 10.35.

### Example 36

1-{[1-Aza-2-oxo-6-(thien-2-yl)]hexyl}-3-{[5-(3,4-dichlorophenyl)-1-oxo-2,3,5-triaza-4-thio]pentyl}benzene (Compound 38; 5c in Scheme XI). To a stirred solution of hydrazide 3c (0.190 g, 0.6 mmol) in DMA (15 mL) was added isothiocyanate 4a (0.086 mL, 0.6 mmol) in one portion. The whole was stirred for 96 h, poured onto ice-water. The residue formed was filtered, washed with water, and air-dried to give Compound 38. Yield 0.177 g (57%). White solid; ¹H NMR (DMSO-d₆, 400 MHz), δ 10.57 (s, 1H); 10.09 (s, 1H); 9.99 (s, 1H); 9.89 (br.s, 1H); 8.10 (s, 1H); 7.83 (s, 2H); 7.49-7.66 (m, 3H); 7.45-7.36 (m, 1H); 7.30 (s, 1H); 6.93 (s, 1H); 6.85 (s, 1H); 2.89-2.77 (m, 2H); 2.43-2.31 (m, 2H); 1.72-1.58 (m, 4H). Anal: Calcd. for C₂₃H₂₂Cl₂N₄O₂S₂: C, 52.97; H, 4.25; N, 10.74. Found: C, 53.40; H, 4.17; 64. N, 10.86.

### Example 37

1-[(6-Phenyl-1-aza-2-oxo)hexyl]-3-{[(adamant-1-yl)-1-oxo-2,3,5-triaza-4-thio]pentyl}-benzene (Compound 41; 5d in Scheme XI). To a stirred solution of hydrazide 3a (0.218 g, 0.7 mmol) in DMA (20 mL) was added isothiocyanate 4d (0.135 g, 0.7 mmol) in one portion. The whole was stirred for 72 h, solvent evaporated in vacuum, and oily residue was recrystallized from aq. EtOH to give Compound 41. Yield 0.105 g (30%). White soild; ¹H NMR (DMSO-d₆, 400 MHz), δ 10.29 (s, 1H); 10.07 (s, 1H); 9.16 (s, 1H); 8.05 (s, 1H); 7.81 (d, J=7.0 Hz, 2H); 7.53 (d, J=7.5 Hz, 1H); 7.39 (t, J=7.8 Hz, 1H); 7.32-7.10 (m, 5H); 2.64-2.56 (m, 2H); 2.39-2.30 (m, 2H); 2.22-2.13 (m, 4H); 2.08-1.89 (m, 6H); 1.67-1.56 (m, 9H). Anal: Calcd. for C₂₉H₃₆N₄O₂S: C, 69.02; H, 7.19; N, 11.10. Found: C, 69.08; H, 7.37; 64. N, 10.56.

### Example 38

1-{[1-Aza-2-oxo-7-(3-trifluoromethylphenyl)]heytyl}-3-{[5-(3 4-dichlorophenyl)-1-oxo-2,3,5-triaza-4-thio]pentyl}benzene (Compound 32; 5e in Scheme XI). To a stirred solution of hydrazide 3e (0.490 g, 1.25 mmol) in DMA (20 mL) was added isothiocyanate 4a (0.207 mL, 1.37 mmol) in one portion. The whole was stirred for 20 h, then poured onto ice-water, and precipitate formed was filtered, then recrystallized from aq. EtOH to give Compound 32. Yield 0.565 g (75%). White soild; ¹H NMR (DMSO-d₆, 400 MHz), δ 1.28-1.41(m, 2H); 1.55-1.72(m, 4H); 2.33(t, J=7.2 Hz, 2H); 2.69(t, J=7.6 Hz, 2H); 7.42(t, J=8 Hz, 1H); 7.47-7.68(m, 7H); 7.77-7.92(m, 2H); 8.11(s, 1H); 9.86-10.13(m, 3H); 10.57(s, 1H). Anal: Calcd. for C₂₇H₂₅Cl₂F₃N₄O₂S: C, 54.28; H, 4.22; N, 9.38. Found: C, 54.23; H, 4.31; 64. N, 9.32.

Compound 42 was prepared according to the following Scheme XII:

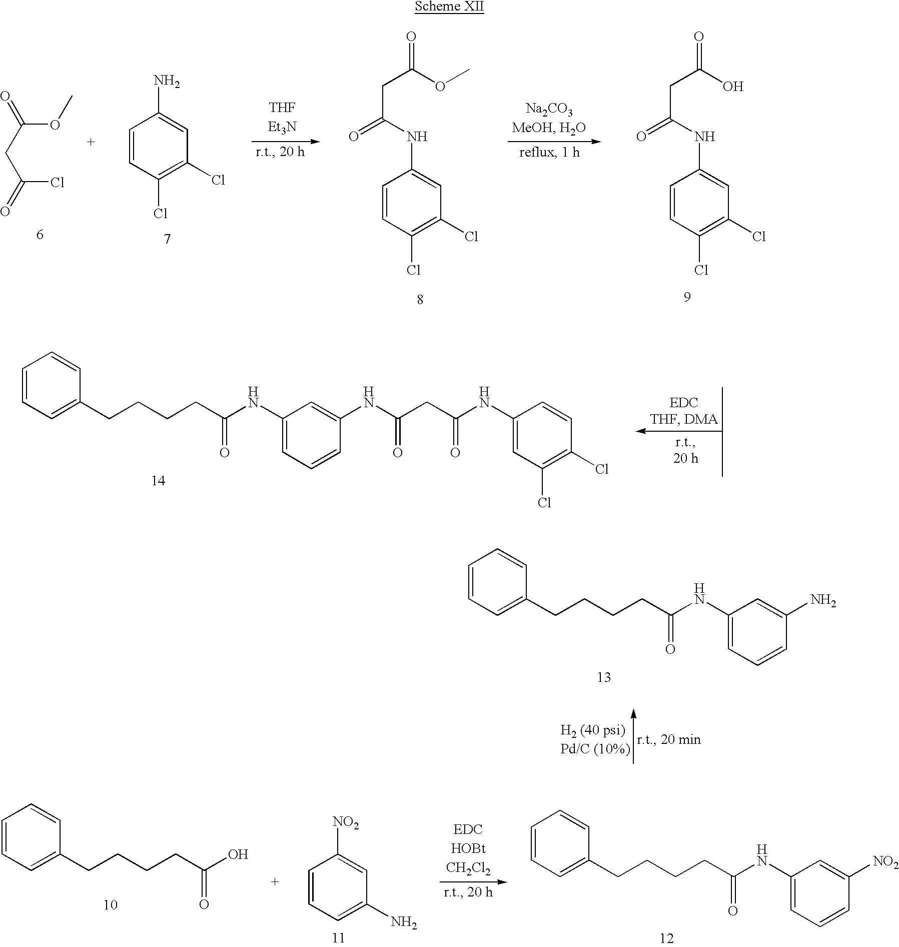

N-(3,4-Dichlorophenyl)malonamic acid methyl ester (8, Scheme XII). A solution of acid chloride 6 (2.14 mL, 20 mmol) in THF (10 mL) was added dropwise within min to a stirred solution of aniline 7 and triethylamine in THF (90 mL) at room temperature and stirring. The temperature gradually rose to 40° C.; precipitation of sticky solid observed. The mixture was stirred at room temperature for 20 h, and then poured onto ice-water. Upon standing for the next 2 days, an oil originally formed gradually crystallized and was then filtered off. Golden-brown prisms. Yield 3.97 g (76%).

N-(3,4-Dichlorophenyl)malonamic acid (9, Scheme XII). A solution of the ester 8 (2.00 g, 7.6 mmol) and Na₂CO₃ in the mixture of methanol (50 mL) and water (30 mL) was stirred and refluxed for 1 h, cooled to room temperature, and diluted with additional amount of water (50 mL). Ethyl acetate (50 mL) was added, and after extraction aqueous layer separated and acidified with HCl conc. to pH 1. The emulsion formed was extracted with ethyl acetate (2×50 mL)/Organic layer separated, dried with MgSO₄ anhyd., filtered, and solvent removed in vacuum to give an oil which crystallized upon standing overnight. Yellow prisms/Yield 1.59 g (84%).

5-Phenyllpentanoic acid (3-nitrophenyl)amide (12, Scheme XII). A solution of amine 11 (0.43 g, 3.09 mmol), acid 10 (0.50 g, 2.81 mmol), HOBt (0.57 g, 4.21 mmol), and EDC (0.81 g, 4.21 mmol) in methylene chloride (10 mL) was stirred at room temperature for 20 h. The whole was taken into ethyl acetate (75 mL), washed subsequently with HCl (1N solution, 25 mL), sodium hydroxide (1N solution, 35 mL), and brine (25 mL). Organic layer separated, dried with MgSO₄ anhyd., filtered, and solvent removed in vacuum to give a yellow solid. Yield 0.90 g (97.8%).

5-Phenylpentanoic acid (3-aminophenyl)amide (13, Scheme XII). Nitro-compound 12 (0.81 g, 2.68 mmol) and Pd/C (0.090 g) in ethanol (10 mL) were shaken in Parr apparatus in H₂ atmosphere at 40 psi. After 20 min, no presence of starting material was observed (TLC), and the suspension filtered through a short-path Celite plug. Ethanol was evaporated in vacuum to give white-gray solid/Yield 0.71 g(99%).

### Example 39

1-[(6-Phenyl-1-aza-2-oxo)hexyl]-3-{[5-(3,4-dichlorophenyl)-1,5-diaza-2,4-oxo]pentyl}-benzene (Compound 42; 14 in Scheme XII). A solution of an acid 9 (0.68 g, 2.5 mmol) and EDC (0.73 g, 3.8 mmol) in the mixture of THF (30 mL) and DMA (15 mL) was stirred for 20 min at room temperature. Amine 13 (0.68 g, 2.5 mmol) was added as a solid, and the stirring was continued for 20 h. Yellow solution formed was poured onto ice-water. Oil formed was solidified upon standing overnight. Tan-yellow microcrystals finally formed were filtered and air-dried. Yield 0.86 g (68%). ¹H NMR (DMSO-d₆, 400 MHz), δ 10.48 (s, 1H); 10.18 (s, 1H); 9.91 (s, 1H): 8.01 (s, 1H); 7.94 (br.s, 1H); 7.59 (d, J=8.6 Hz, 1H); 7.50 (d, J=8.8 Hz, 1H); 7.33-7.13 (m, 8H); 3.48 (s, 2H); 2.64-2.55 (m, 2H); 2.37-2.28 (m, 2H); 1.62-1.53 (m, 4H). Anal: Calcd. for C₂₆H₂₅Cl₂N₃O₃(0.55 H₂O): C, 61.44; H, 5.18; N, 8.27. Found: C, 61.40; H, 5.26; 64. N, 8.13.

Compound 40A was prepared according to the following Scheme XIII:

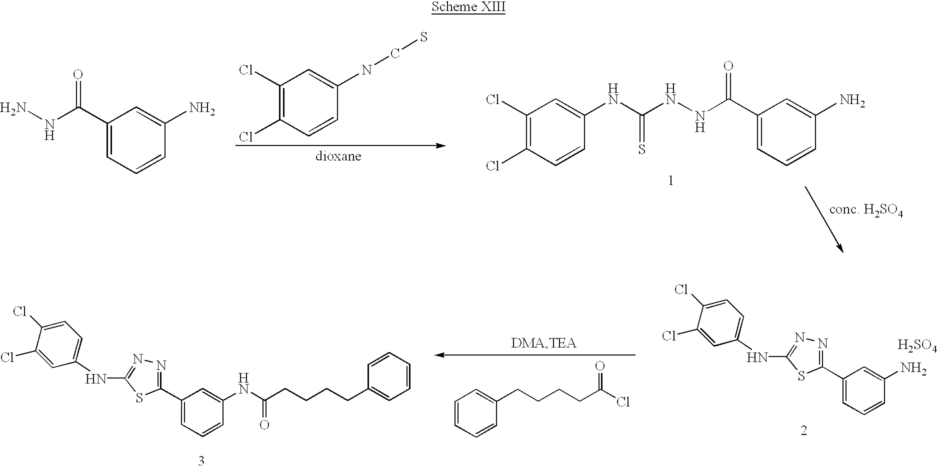

(3-aminophenyl)-N-({[3,4-dichlorophenyl)amino]thioxomethyl}amino)carboxamide (1, Scheme XIII). 1,2-Dichloro-4-isothiocyanato-benzene (4.7 g, 23.15 mmol) dissolved in hexanes (25 mL) was added dropwise to a stirring solution of 3-amino-benzoic acid hydrazide (3.5 g, 23.15 mmol) in dioxane (150 mL) and stirred overnight. The reaction mixture was filtered through celite and concentrated to half the volume. The solution was diluted with hexanes (50 mL) and allowed to crystallize overnight at 4° C. The solution was filtered to provide 6.0 g (72.9%) of desired product as a white solid. ¹H NMR (DMSO-d₆, 400 MHz) δ 5.31 (brs, 2H); 6.73-6.76 (m, 1H); 7.10-7.13 (m, 3H); 7.55-7.59 (m, 2H); 7.82 (brs, 1H); 9.86 (brs, 1H); 9.92 (brs, 1H); 10.35 (s, 1H).

[5-(3-Amino-phenyl)-[1,3,4]thiadiazol-2-yl]-(3,4-dichloro-phenyl)-amine•sulfate (2, Scheme XIII) (3-aminophenyl)-N-({[3,4-dichlorophenyl)amino]thioxomethyl}amino)carboxamide (2.0 g, 5.63 mmol) was suspended in concentrated H₂SO₄ and let stir for 1.5 hours. The mixture was poured over ice/water resulting in a yellow precipitate. The solution was filtered to provide 2.2 g (91.6%) of desired product as a yellow solid. ¹H NMR (DMSO-d₆, 400 MHz) δ 4.82 (brs, 2H); 7.08 (d, 1H); 7.38-7.41 (m, 2H); 7.50-7.55 (m, 2H); 7.60-7.63 (m, 1H); 8.15 (s, 1H); 10.90 (s, 1H).

### Example 40

5-Phenyl-pentanoic acid {3-[5-(3,4-dichloro-phenylamino)-[1,3,4]thiadiazol-2-yl]-phenyl}-amide (Compound 40A; 3 in Scheme XIII). 5-Phenyl-pentanoyl chloride (0.08 g, 0.39 mmol) dissolved in DMA (2 mL) was added dropwise to a stirring solution of [5-(3-Amino-phenyl)-[1,3,4]thiadiazol-2-yl]-(3,4-dichloro-phenyl)-amine•H₂SO₄ (0.1 g, 0.3 mmol) and triethyl amine (0.08 g, 0.76 mmol) dissolved in DMA (2 mL) and stirred overnight. The reaction mixture was washed with water. The organic phase was dried over MgSO₄ and concentrated to a yellow oil. The oil was dissolved in EtOAc to yield a white precipitate. The solution was filtered to provide 0.45 g (30.2%) of desired product as a white solid. ¹H NMR (DMSO-d₆, 400 MHz) δ 1.63 (m, 4H); 2.37 (m, 2H); 2.62 (m, 2H); 7.17-7.23 (m, 3H); 7.26-7.34 (m, 2H); 7.43-7.45 (m, 1H); 7.51-7.53 (m, 2H); 7.62 (d, 1H); 7.70 (d, 1H); 8.14 (s, 1H); 8.19 (s, 1H); 10.13 (s, 1H); 10.88 (s, 1H). TLC: R_{f}=0.5 (50% EtOAc/hexane) MS: (ES+): 498.

Compound 39 was prepared according to the following Scheme XIV:

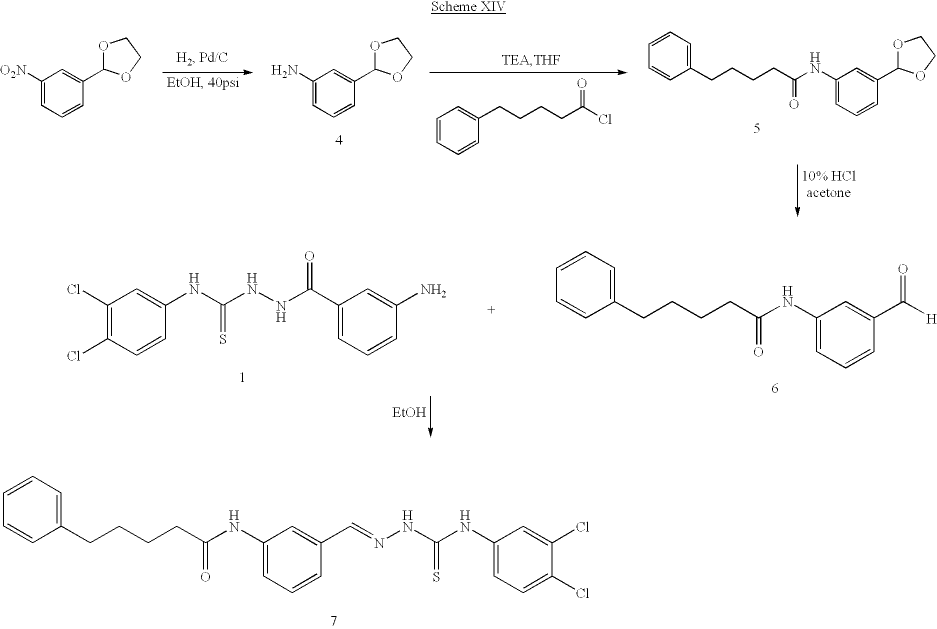

3-[1,3]Dioxolan-2-yl-phenylamine (4, Scheme XIV). (10%) Palladium on carbon (0.25 g) was suspended in a solution of 2-(3-Nitro-phenyl)-[1,3]dioxolane (2.5 g, 12.82 mmol) dissolved in ethanol (15 mL). The solution was hydrogenated on a Parr shaker at 40 psi for 1 hour. The solution was filtered through a celite plug and concentrated to provide 0.19 g (90.0%) of desired product as a clear oil. ¹H NMR (DMSO-d₆, 400 MHz) δ 3.88-4.01 (m, 4H); 5.11 (brs, 2H); 5.55 (s, 1H); 6.53 (s, 1H); 6.55 (s, 1H); 6.64 (s, 1H); 7.00 (t, 1H, J=8.0 Hz).

5-Phenyl-pentanoic acid (3-[1,3]dioxolan-2-yl-phenyl)-amide (5, Scheme XIV). 5-Phenyl-pentanoyl chloride (0.19 g, 1.00 mmol) dissolved in THF (1 mL) was added dropwise to a stirring solution of 3-[1,3]Dioxolan-2-yl-phenylamine (0.17 g, 1.00 mmol) and triethyl amine (0.15 g, 1.50 mmol) dissolved in THF (4 mL) and stirred overnight. The reaction mixture was washed with water. The organic phase was dried over MgSO₄ and concentrated to a yellow oil. The oil was purified on a radial chromatatron (30% EtOAc/hexane) to yield 0.3 g (92.3%) of desired product as a light yellow oil. ¹H NMR (DMSO-d₆, 400 MHz) δ 1.58-1.62 (m, 4H); 2.33 (m, 2H); 2.61 (m, 2H); 4.00 (m, 4H); 5.68 (s, 1H); 7.07-7.34 (m, 7H); 7.58 (s, 1H); 7.70 (s, 1H); 9.95 (s, 1H).

5-Phenyl-pentanoic acid (3-formyl-phenyl)-amide (6, Scheme XIV)._{—}10% Hydrochloric acid (3 mL) was added dropwise to a stirring solution of 5-Phenyl-pentanoic acid (3-[1,3]dioxolan-2-yl-phenyl)-amide (0.3 g, 0.92 mmol) dissolved in acetone (5 mL) and let stir for 30 minutes at an ambient temperature. The solution was cooled to 4° C. and let stir overnight. The solution was concentrated to yield 0.26 g (100%) of desired product as a light yellow oil. ¹H NMR (DMSO-d₆, 400 MHz) δ 1.58-1.63 (m, 4H); 2.33 (m, 2H); 2.61 (m, 2H); 7.19-7.29 (m, 6H); 7.51-7.60 (m, 2H); 7.86 (m, 1H); 8.24 (s, 1H); 9.96 (s, 1H); 10.39 (s, 1H).

### Example 41

N-{3-[(1E)-2-aza-2-({[(3,4-dichlorophenyl)amino]thioxomethyl}amino)vinyl]phenyl}-5-phenylpentanamide (Compound 39; 7 in Scheme XIV). 5-Phenyl-pentanoic acid (3-formyl-phenyl)-amide (0.26 g, 0.93 mmol) dissolved in ethanol (5 mL) was added to a stirring solution of (3-aminophenyl)-N-({[(3,4-dichlorophenyl)amino]thioxomethyl}amino)carboxamide (0.22 g, 0.93 mmol) dissolved in ethanol and was refluxed for 1 hour at 90° C. The solution was cooled to an ambient temperature to yield yellow crystals. The solution was filtered to provide 0.25 g (54.3%) of desired product as a yellow solid. ¹H NMR (DMSO-d₆, 400 MHz) δ 1.58-1.63 (m, 4H); 2.33 (m, 2H); 2.61 (m, 2H); 7.15-7.20 (m, 3H); 7.25-7.29 (m, 2H); 7.36 (t, 1H, J=8.0 Hz); 7.60-7.69 (m, 3H); 7.76 (d, 1H, J=8.0 Hz); 7.87 (brs, 1H); 7.99 (d, 1H, J=4.0 Hz); 8.13 (s, 1H); 10.00 (s, 1H); 10.19 (s, 1H); 12.03 (s, 1H). MS: (ES+): 499.

Compound 40B was prepared according to the following Scheme XV:

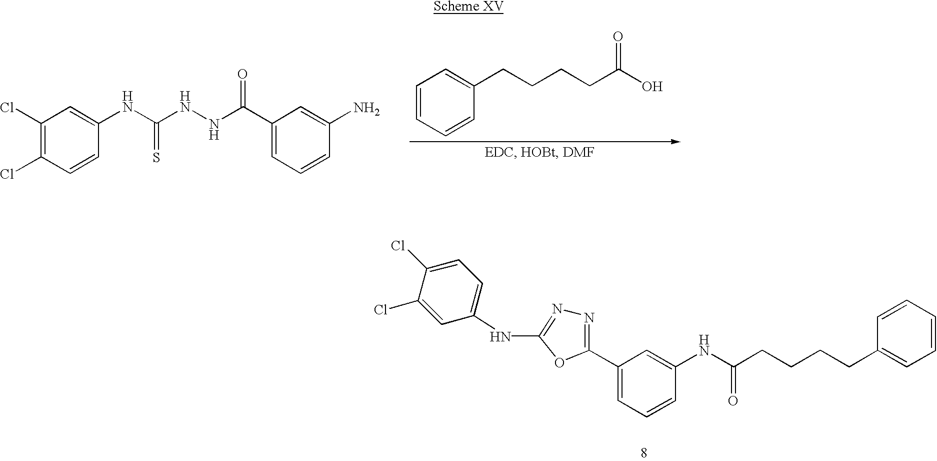

### Example 42

5-Phenyl-pentanoic acid {3-[5-(3,4-dichloro-phenylamino)-[1,3,4]oxadiazol-2-yl]-phenyl}-amide (Compound 40B; 8 in Scheme XV)._{—}5-Phenyl-pentanoic acid (0.1 g, 0.56 mmol), 1-[3-(dimethylamino)propyl]-3-ethylcarbodiimide hydrochloride (0.16 g, 0.84 mmol), (3-aminophenyl)-N-({[(3,4-dichlorophenyl)amino]thioxomethyl}amino)carboxamide (0.2 g, 0.56 mmol) and 1-hydroxybenzotriazole (0.11 lg, 0.84 mmol) was dissolved in anhydrous DMF (10 mL) and stirred overnight. The reaction mixture was concentrated to a yellow oil. The oil was purified on a silica gel column (50% EtOAc/hexane) to yield 0.08 g (31%) of desired product as a white solid. Acidic conditions results in the cyclized product. ¹H NMR (DMSO-d₆, 400 MHz) δ 1.63 (m, 4H); 2.36 (m, 2H); 2.62 (m, 2H); 7.15-7.21 (m, 3H); 7.26-7.30 (m, 2H); 7.47-7.64 (m, 5H); 7.95 (s, 1H); 8.40 (s, 1H); 10.18 (s, 1H); 11.14 (s, 1H). TLC: R_{f}=0.5 (50% EtOAc/hexane) MS: (ES+): 481.

Compound 43 was prepared according to the following Scheme XVI:

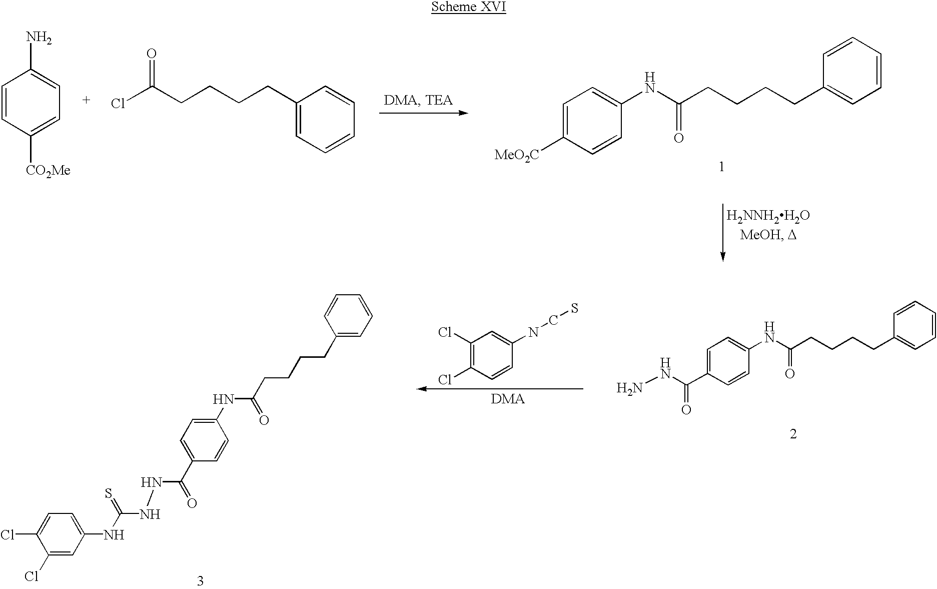

Methyl 4-(5-Phenylipentanoylamino)benzoate (1, Scheme XVI). Triethylamine (5 mL, 36 mmol) was slowly added to a solution of methyl 4-aminobenzoate (2.0 g, 13.2 mmol) and 5-phenylpentanoyl chloride (2.6 g, 13.2 mmol) in DMA (25 mL) and let stir overnight. The mixture was poured over ice and the resulting off-white solid filtered and let dry. Recrystallized from EtOAc/hexane to give 2.1 g (50%) of desired product as fine off-white needles (mp. 108-111° C.). ¹H NMR (DMSO-d₆, 400 MHz) δ 1.59-1.63 (m, 4H); 2.36-2.39 (m, 2H); 2.59-2.62 (m, 2H); 3.81 (s, 3H); 7.15-7.29 (m, 5H); 7.72 (d, 2H, J=8.8 Hz); 7.90 (d, 2H, J=8.8 Hz); 10.24 (s, 1H).

4-(5-Phenylpentanoylamino)benzhydrazide (2, Scheme XVI). A solution of methyl 4-(5-phenylpentanoylamino)benzoate (1, 1.0 g, 3.2 mmol) in methanol (25 mL) containing hydrazine hydrate (0.8 g, 16 mmol) was refluxed for 48 hrs. The mixture was evaporated and the residue partitioned between EtOAc/H₂O. The organic phase was washed with brine, dried with anhydrous MgSO₄, filtered and evaporated to give 0.95 g (95%) white solid. ¹H NMR (DMSO-d₆, 400 MHz) δ 1.59-1.61 (m, 4H); 2.34-2.37 (m, 2H); 2.59-2.62 (m, 2H); 4.46 (bs, 2H); 7.15-7.29 (m, 5H); 7.63 (d, 2H, J=8.6); 7.76 (d, 2H, J=8.8); 9.63 (s, 1H); 10.09 (s, 1H).

### Example 43

1-{4-[(5-Phenyl)pentanoylamino]benzoyl}-4-(3,4-dichlorophenyl)thiosemicarbazide (Compound 43; 3 in Scheme XVI). A solution of 4-(5-phenylpentanoylamino)benzhvdrazide (2, 0.1 g, 0.32 mmol) and 3,4-dichlorophenyl isothiocyanate (0.65 g, 0.32 mmol) in DMA (5 mL) was stirred overnight. The mixture was poured on ice and the resulting white solid collected, triturated with dichloromethane and dried in vacuo to give 0.05 g (31%) white solid. ¹H NMR (DMSO-d₆, 400 MHz) δ 1.59-1.63 (m, 4H); 2.37-2.39 (m, 2H); 2.59-2.62 (m, 2H); 7.15-7.29 (m, 5H); 7.53-7.60 (m, 2H); 7.69 (d, 2H, J=8.8); 7.82 (bs, 1H); 7.89 (d, 2H, J=8.8); 9.88 (bs, 1H); 9.95 (s, 1H); 10.17 (s, 1H); 10.46 (s, 1H). Anal. Calcd. for C₂₅H₂₄Cl₂N₄O₂S: C, 58.25; H, 4.69; N, 10.87; S, 6.22; Cl, 13.76. Found: C, 58.10; H, 4.74; N, 10.78; S, 6.26; Cl, 13.80.

Compounds 34A and 34B were prepared according to the following Scheme XVII:

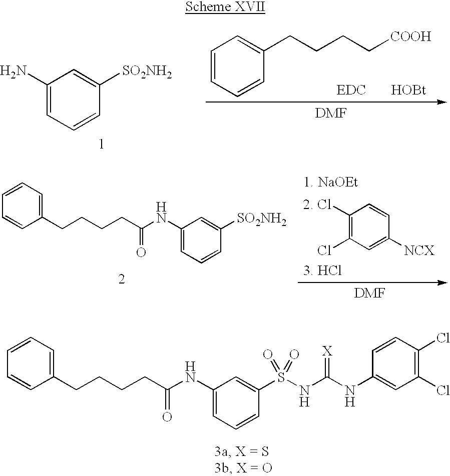

5-Phenyl-pentanoic acid (3-sulfamoyl-phenyl)-amide (2, Scheme XVII). To a solution of 5-phenylvaleric acid (3.1 g, 17 mmol) in 80 mL DMF was added 1-[3-(dimethylamino)propyl]-3-ethylcarbodiimide hydrochloride (4.3 g, 23 mmol) and 1-hydroxybenzotriazole (3.0 g, 23 mmol) and the mixture stirred 1 h. at r.t. To this was then added 3-aminobenzenesulfonamide and the mixture stirred 3d. at r.t. The mixture was then concentrated and the product purified on silica with 2:3 hexane:ethyl acetate to give 2 as a white solid, 5.1 g (88%): ¹H NMR (CDCl3, 400 MHz): δ 1.54-1.71(m, 4H); 2.31-2.43(m, 2H); 2.56-2.69(m, 2H); 7.12-7.40(m, 7H); 7.45-7.54(m, 2H); 7.69-7.79(m, 1H); 8.17(s, 1H); 10.17(s, 1H). TLC: R_{f}=0.6 (2:1 EtOAc:Hexane).

### Example 44

1-[3-(6-Phenylpentanoylamino)-benzenesulfonyl]-3-(3,4-dichlorophenyl)thiourea (Compound 34A, 3a in Scheme XVII). To a solution of 2 (0.43 g, 1.3 mmol) in 10 mL DMF was added 21 wt. % sodium ethoxide solution in ethanol (0.5 mL, 1.3 mmol) and the mixture stirred 3 h. at 85° C. The mixture was then allowed to cool to r.t., treated with 3,4-dichlorophenylisothiocyanate (0.27 g, 1.3 mmol) and stirred overnight. At this time, the mixture was neutralized with 4M hydrogen chloride solution in dioxane (0.33 mL, 1.3 mmol). The reaction mixture was then concentrated and the product purified on silica with 100% ethyl acetate to a yellow oil which was recrystallized in hexanes:ethyl acetate to give Compound 34A as a white solid, 0.15 g (22%): ¹H NMR (CDCl3, 400 MHz): δ 1.52-1.71(m, 4H); 2.27-2.42(m, 2H); 2.56-2.67(m, 2H); 7.14-7.47(m, 9H); 7.63(dd, J=2.5,8.8 Hz, 1H) 7.77(d, J=7.8 Hz, 1H); 7.87(s, 1H); 8.22(d, J=2.5 Hz, 1H); 9.34(s, 1H); 10.03 (s, 1H). Anal. Calcd for C₂₄H₂₃N₃S₂Cl₂O₃.1.6H₂O: C, 50.99; H, 4.67; N, 7.43. Found: C, 50.96; H, 4.67; N, 7.43. TLC: R_{f}=0.2 (100% EtOAc).

### Example 45

1-[3-(6-Phenylpentanoylamino)-benzenesulfonyl]-3-(3,4-dichlorophenyl)urea (Compound 34B, 3b in Scheme XVII). The procedure was carried out as noted for 3a, using 3,4-dichlorophenylisocyanate in place of 3,4-dichlorophenylisothiocyanate, which yielded Compound 34B as a white solid, 0.52 g (77%): ¹H NMR (CDCl3, 400 MHz): δ 1.52-1.71(m, 4H); 2.27-2.41(m, 2H); 2.55-2.67(m, 2H); 7.10-7.41(m, 9H); 7.49(d, J=7.6 Hz, 1H); 7.75(d, J=7.3 Hz, 1H); 7.84(s, 1H); 8.05(s, 1H); 8.94(s, 1H); 10.10(s, 1H). Anal. Calcd for C₂₄H₂₃N₃S₁Cl₂O₄.1.0H₂O: C, 53.36; H, 4.70; N, 7.78; S, 5.94; Cl, 13.12. Found: C, 53.11; H, 4.37; N, 7.88; S, 5.88: Cl, 12.92. TLC: R_{f}=0.2 (100% EtOAc).

Compound 35 was prepared according to the following Scheme XVIII:

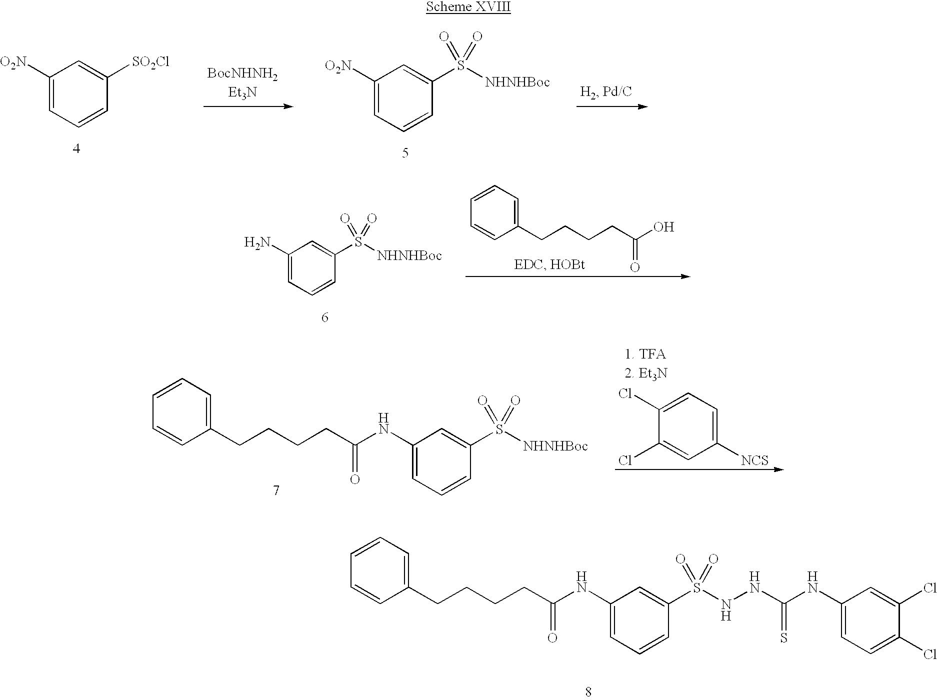

1-(3-Nitrobenzenesulfonyl)-2-(t-butyloxycarbonyl)hydrazine (5, Scheme XVIII). To a solution of tert-butylcarbazate (23.2 g, 175 mmol) and triethylamine (3.32 g, 33 mmol) in 250 mL dichloromethane under argon and cooled to 0° C. in an ice bath was added dropwise a solution of 3-nitrobenzenesulfonyl chloride (4, 5.00 g, 21.9 mmol) and the mixture stirred overnight allowing it to warm to r.t. The mixture was then concentrated and the product purified on silica with 99:1 chloroform:methanol to give 5 as a white solid, 3.7 g (52%): ¹H NMR (DMSO, 400 MHz): δ 1.20(s, 9H); 7.90(t, J=7.8 Hz, 1H); 8.20(d, J=7.8 Hz, 1H); 8.46-8.55(m, 2H); 9.41(bs, 1H); 10.04(s, 1H). TLC: R_{f}=0.3 (98:2 chloroform:methanol).

1-(3-Aminobenzenesulfonyl)-2-(t-butyloxycarbonyl)hydrazine (6, Scheme XVIII). A solution of 5 (1.5 g, 4.7 mmol) in 15 mL of methanol containing 0.3 g 10% palladium on carbon was hydrogenated at 50 psi hydrogen for 1 h. The mixture was filtered through celite and the filtrate concentrated to give pure 6 as a white solid, 1.4 g (99%): ¹H NMR (DMSO, 400 MHz): δ 1.27(s, 9H); 5.52(s, 2H); 6.74(d, J=7.6 Hz, 1H); 6.86(d, J=7.6 Hz, 1H); 6.94-7.03(m, 1H); 7.14(t, J=7.6 Hz, 8.61-9.18(m, 1H); 9.28(s, 1H). TLC: R_{f}=0.15 (98:2 chloroform:methanol).

1-[3-(5-Phenylpentanoylamino)benzenesulfonyl]-2-(t-butyloxvcarbonyl)hydrazine (7, Scheme XVIII). To a solution of 5-phenylvaleric acid (0.75 g, 4.2 mmol) in 20 mL DMF was added 1-[3-(dimethylamino)propyl]-3-ethylcarbodiimide hydrochloride (1.0 g, 5.4 mmol) and 1-hydroxybenzotriazole (0.73 g, 5.4 mmol) and the mixture stirred 1 hr. At this time, 6 (1.2 g, 4.2 mmol) was added and the mixture stirred 2d. The mixture was then concentrated and the product purified on silica with 99:1 chloroform:methanol to give 7 as a white solid, 1.2 g (64%): ¹H NMR (DMSO, 400 MHz): δ 1.21(s, 9H); 1.53-1.75(m, 4H); 2.29-2.44(m, 2H); 2.56-2.68(m, 2H); 7.12-7.35(m, 5H); 7.37-7.55(m, 2H); 7.83(d, J=7.1 Hz, 1H); 8.11(s, 1H); 8.70-9.30(m, 1H); 9.53(s, 1H); 10.20(s, 1H). TLC: R_{f}=0.2 (98:2 chloroform:methanol).

### Example 46

1-[3-(6-Phenylpentanoylamino)-benzenesulfonyl]-4-(3,4-dichlorophenyl)thiosemicarbazide (Compound 35, 8 in Scheme XVIII). To a solution of 7 (0.5 g, 1.1 mmol) in 4 mL dichloromethane was 6 mL trifluoroacetic acid and the mixture stirred 1d. The mixture was then concentrated and residual trifluoroacetic acid removed under high vacuum. The residue was dissolved in 10 mL dichloromethane, neutralized with triethylamine (0.34 g, 3.4 mmol) and then treated with 3,4-dichlorophenylisothiocyanate and stirred 1d. The mixture was concentrated and the product purified on silica with 96:4 chloroform:methanol to a yellow oil which was recrystallized in ethyl acetateihexane to give Compound 35 as a white solid, 0.10 g (17%): ¹H NMR (DMSO, 400 MHz): δ 1.54-1.68(m, 4H); 2.31-2.42(m, 2H); 2.57-2.66(m, 2H); 7.12-7.31(m, 5H); 7.43-7.58(m, 4H); 7.44(d, J=2.3 Hz, 1H); 7.86(d, J=7.3 Hz, 1H); 8.18(s, 1H); 9.95(s, 1H); 10.06(s, 1H); 10.13(s,1H); 10.25(s, 1H). Anal. Calcd for C₂₄H₂₄N₄S₂Cl₂O₃: C, 52.27; H, 4.39; N, 10.16; S, 11.63; Cl, 12.86. Found: C, 52.32; H, 4.49; N, 10.15; S. 11.36: Cl, 12.58. TLC: R_{f}=0.3 (96:4 chloroform: methanol).

Compound 36 was prepared according to the following Scheme XIX.

Ethyl 3-(6-phenylhexanoylamino)benzoate (9, Scheme XIX). A solution of 3-aminobenzoic acid ethyl ester (0.34 g, 2.1 mmol), 6-phenylhexanoic acid (0.40 g, 2.1 mmol), 1-[3-(dimethylamino)propyl]-3-ethylcarbodiimide hydrochloride (0.52 g, 2.7 mmol) and 1-hydroxybenzotriazole (0.37 g, 2.7 mmol) in 10 mL DMF was stirred 2d. The mixture was concentrated and the product purified on silica with 3:1 hexane:ethyl acetate to give 9 as a white solid. Yield 0.61 g (87%): TLC: R_{f}=0.6 (2:1 hexane:ethyl acetate).

3-(6-Phenylhexanoylamino)benzoic acid (10, Scheme XIX). To a solution of 9 (0.7 g, 2.1 mmol) in 20 mL of ethanol was added a solution of lithium hydroxide monohydrate (95 mg, 2.3 mmol) in 2 mL of water, and the mixture was stirred overnight. The mixture was then diluted with 50 mL of water, acidified to pH 2 with 1N HCI and extracted with three 50 mL portions of chloroform. The combined organic portions were dried over magnesium sulfate, filtered and concentrated to give 10 as a white solid, 0.62 g (97%).

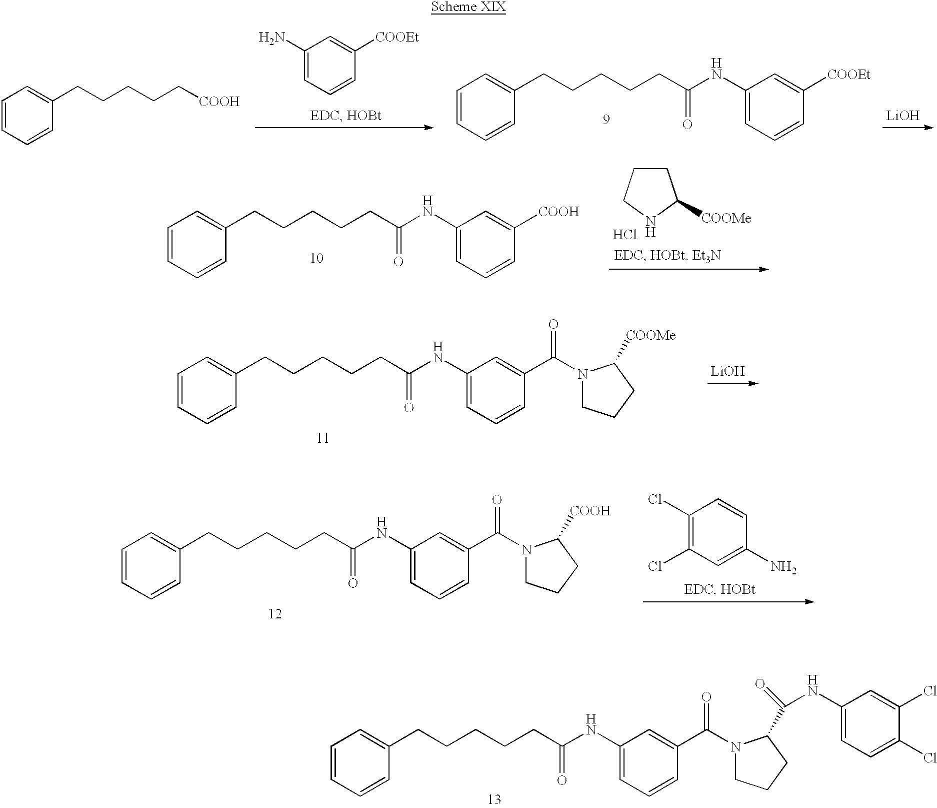

L-N-[3-(6-Phenylhexanoylamino)benzoyl]proline methyl ester (11, Scheme XIX). To a solution of L-proline methyl ester hydrochloride (0.32 g, 1.9 mmol) in 10 mL DMF was added triethylamine (0.34 g, 3.4 mmol), followed by 10 (0.53 g, 1.7 mmol), 1-[3-(dimethylamino)propyl]-3-ethylcarbodiimide hydrochloride (0.42 g, 2.2 mmol), and 1-hydroxybenzotriazole (0.30 g, 2.2 mmol). The mixture was stirred for 3 days. It was then concentrated in vacuum, and the product purified on silica gel with 1:2 hexane:ethyl acetate to give 11 as a white solid, 0.60 g (83%): TLC: R_{f}=0.3 (1:2 hexane:ethyl acetate).

L-N-[3-(6-Phenylhexanoylamino)benzoyl]proline (12, Scheme XIX). To a solution of 11 (0.6 g, 1.4 mmol) in 20 mL methanol was added a solution of lithium hydroxide monohydrate (90 mg, 2.1 mmol) in 2 mL water, and the mixture stirred overnight. The mixture was then diluted with 50 mL water, acidified to pH 2 with 1 N HCl and extracted with three 50 mL portions of chloroform. The combined organic portions were dried with magnesium sulfate, filtered and concentrated to give 12 as a white solid, 0.56 g (96%).

### Example 47

L-N-[3-(6-Phenylhexanoylamino)benzoyl]proline 3,4-dichlorobenzamide (Compound 36; 13 in Scheme XIX). A solution of 12 (0.56 g, 1.4 mmol), 3,4-dichloroaniline (0.24 g, 1.5 mmol), 1-[3-(dimethylamino)propyl]-3-ethylcarbodiimide hydrochloride (0.34 g, 1.8 mmol) and 1-hydroxybenzotriazole (0.24 g, 1.8 mmol) in 10 mL DMF was stirred for 3 days. The mixture was concentrated and the product purified on silica with 1:2 hexane:ethyl acetate to give Compound 36 as a white solid, 0.61 g (87%): ¹H NMR (DMSO, 400 MHz): δ 1.21-1.40(m, 2H); 1.47-1.71(m, 4H); 1.79-2.03(m, 3H); 2.20-2.38(m, 3H); 2.53-2.66(m, 2H); 3.45-3.68(m, 2H); 4.56(dd, J=8.1, 5.3 Hz, 1H); 7.10-7.31(m, 6H); 7.33-7.42(m, 1H); 7.44-7.69(m, 3H); 7.87(s, 1H); 8.06(s, 1H); 10.02(s, 1H); 10.45(s, 1H). Anal. Calcd for C₃₀H₃₁N₃Cl₂O₃: C, 65.22; H, 5.66; N, 7.61; Cl, 12.83. Found: C, 64.92; H, 5.64; N, 7.55; Cl, 12.66. TLC: R_{f}=0.6 (2:1 hexane:ethyl acetate).

### Exemplary Ways to Detect Binding to a CyP

### Example 48

Measuring the Inhibition of Rotamase (Prolyl Peptidyl cis-trans Isomerase) Activity

A number of substrates for rotamase are known in the art or can be derived from those known. Typically, the substrate contacts a sample containing a protein with rotamase activity and the conversion of the substrate is detected after a period of time. The method for detecting conversion of the substrate will vary with the particular substrate chosen. One method has been termed the Kᵢ test (See Harding, et al., Nature, 341:758-760 (1989). The cis-trans isomerization of an alanine-proline bond in a model substrate, N-succinyl-Ala-Ala-Pro-Phe-p-nitroanilide, is monitored spectrophotometrically in a chymotrypsin-coupled assay. The action of chymotrypsin releases p-nitroaniline from only the trans form of the substrate. The amount of p-nitroaniline can be monitored in a spectrophotometer, for example. Other methods of detecting the presence of p-nitroaniline can also be used. The inhibition of this reaction caused by different concentrations of inhibitor is determined and the data are analyzed as a change in first-order rate constant as a function of inhibitor concentration, which yields the Kᵢ value.

The following were added to a plastic cuvette: 950 mL of ice cold assay buffer (25 mM HEPES, pH 7.8, 100 mM NaCl), 10 μL of CyP A (2.5 μM in 10 mM Tris-Cl pH 7.5, 100 mM NaCl, 1 mM dithiothreitol), 25 μL of chymotrypsin (50 mg/ml in 1 mM HCl), and 10 μL of test compound, at various concentrations, in dimethyl sulfoxide. The reaction was initiated by the addition of 5 μL of substrate (succinyl-Ala-Phe-Pro-Phe-para-nitroanilide, 5 mg/mL in 470 mM LiCl in trifluoroethanol). The absorbance at 390 nm versus time was monitored for 90 seconds using a spectrophotometer and the rate constants were determined from the absorbance versus time data files.

The IC₅₀ values that were obtained for representative compounds in the following Table I refer to the concentration that inhibits 50% of the rotamase activity in a sample. The lower the value, the more active the compound is at binding or interacting with CyP. The Cyclophilin utilized was a recombinant rat CyPA-GST fusion protein: CypA was amplified from rat brain cDNA using standard PCR methods, primed with the following sequences: 5′ CCC CCC GGG AGT CAA CCC CAC CGT GTT CTT CGA 3′ and 5′ GGA GAT CTA GAG TTG TCC ACA GTC GGA GAT GGT 3′. The resulting fragment (573 base pairs) was cloned into pCRII and amplified. The CyP sequence was cut out with Smal and EcoRl and cloned into the Smal/EcoRl sites in pGEX2TK (Pharmacia). This plasmid was transformed into BL21 E. coli cells for expression of the GST-CyPA fusion protein. An asterisk indicates that the compound was evaluated using a human recombinant CyPA, [Yoo et al., J. Mol. Biol., 269 (1997) 780-95].

While CyP A was used in these examples, other CyP proteins can be substituted. Similar methods can be used with other immunophilins, such as the FKBPs, to demonstrate the presence or absence of FKBP binding activity. Preferred compounds have an IC₅₀≦1 μM for inhibition of cyclophilin rotamase activity. Especially preferred compounds may also have an IC₅₀≧500 nM for inhibition of FKBP rotamase activity.

TABLE I

| | IC₅₀ | | IC₅₀ |
|---|---|---|---|
| Compound # | (nM) | Compound # | (nM) |
| 1 | 162 | 16 | 1530 |
| 2 | 149 | 17 | 854 |
| 3 | 955 | 18 | 901 |
| 4 | 995 | 19 | 754 |
| 5 | 783 | 20 | 188 |
| 6 | 990 | 21 | 512* |
| 7 | 1220 | 22 | 733* |
| 8 | 701/804* | 23 | 895 |
| 8a | 364 | 24 | 909 |
| 9 | n.d. | 25 | 964 |
| 10 | 732 | 26 | 1560 |
| 11 | 2060* | 27 | 667 |
| 12 | 767 | 28 | 1100 |
| 13 | 680 | 29 | 940 |
| 14 | 786 | 30 | 464* |
| 15 | 610 | 31 | 974* |
| 15a | 610/521* | 33 | 85* |

### Example 49

Semiautomated Assay of Rotamase Activity Inhibition: Rotamase inhibition was determined using a semiautomated microtiter plate assay modification of the above described assay [see Küillertz et al., Clin. Chem. 44, 502-508 (1998)]. All dilutions of CyPA, peptide substrate, and chymotrypsin were made in 35 mM ice-cold HEPES buffer. Fifty μL of CyPA solution were added to 50 μL of peptide substrate solution (Phe-Pro-Phe p-nitroanilide, 0.16 mg/ml) in a glass microtiter well plate (the concentration of human recombinant CyP being adjusted so that the reaction rate is increased by a factor of 3 as compared to an uncatalyzed control reaction wherein the peptide substrate is degraded by chymotrypsin alone, in the absence of CyPA and test compound). Using a Beckman Multimek™ 96 automated 96-channel pipettor, 5 μL of test compound solution, or a DMSO blank, were added to each well for a 30 minute preincubation at 4° C. One hundred μL of chymotrypsin solution (1 mg/mL) were added to each well and the absorbance at 390 nm versus time was monitored for 9-10 minutes using a BioRad Ultramark plate reader maintained at 4° C. and the rate constants were determined from the absorbance versus time data files. The cyclophilin utilized was a human recombinant CyPA [Yoo et al., J. Mol. Biol., 269 (1997) 780-95].

IC₅₀ values which were obtained for representative compounds using this modified plate reader assay of rotamase inhibition are given in the following Table II:

TABLE II

| | IC₅₀ | | IC₅₀ |
|---|---|---|---|
| Compound # | (nM) | Compound # | (nM) |
| 9 | 309 | 36 | 9050 |
| 13 | 2060 | 37 | 532 |
| 13a | 4180 | 38 | 672 |
| 30 | 3760 | 39 | 1410 |
| 31 | 2710 | 40A | 2800 |
| 32 | 154 | 40B | 883 |
| 33 | 713 | 41 | 5190 |
| 34A | 4640 | 42 | 6090 |
| 34B | 19100 | 43 | 1570 |
| 35 | 372 | 44 | 80 |
| | | 45 | 130 |

### Measuring the Neuroactivity of the Compounds of the Invention

As noted above, a number of methods can be used to assay for the bioactivity of the compounds of the invention. These assays can be in vivo or in vitro methods. The examples below illustrate assays for the ability of the compounds to protect neuronal cells from toxic treatments and the ability of the compounds to elicit neuronal cell growth, regeneration, or neurite extension.

### Example 50

Immunostaining and Neurite Outgrowth Quantitation in Dorsal Root Gangila Preparations: Dorsal root ganglia (DRG) from adult mice can be isolated by micro-dissection. The spinal cord with attached DRGs from an adult mouse (15-10 g) is removed. Spinal nerves are cut away using micro-dissection scissors and any excess material is trimmed until the DRG is free. Using sharp micro-dissecting scissors, a transverse cut is made in the peripheral nerve, leaving 1-2 mm attached, and the explant placed into Petri dish and covered with plating media. When finished collecting all DRGs, the spinal nerve is trimmed to about 1 mm in length. The explant is then embedded in 30 μl of reduced growth factor Matrigel on a circular coverslip, and placed in a 35 mm culture dish. The sensory ganglion explant is then covered with 2 ml of media. Compounds, drugs or control solutions are added from 10× stocks, and incubated at 37° C., 5% CO₂, 95% humidity for 48 hrs. Cultures are washed twice with PBS, and fixed with 10% formalin for 30 minutes. Fixed cultures are rinsed twice with PBS and stored in PBS under refrigeration pending staining and analysis.

For staining, cultures are incubated in Block Buffer (5% Horse Serum, 5% Goat Serum, 1% Triton X, PBS pH=7.4) overnight, while rotating, at a temperature of 4° C. A primary antibody against beta tubulin (Sigma Chemical Co.) is diluted in Block Buffer and cultures are incubated overnight at 4° C. Preparations are washed 5 times with PBS and a secondary antibody (Alexa 488 Goat Anti-Mouse), diluted in block buffer, is applied overnight at 4° C. Preparations are washed 5 times with PBS and left overnight at 4° C. Cultures are coverslipped and total neurite length from the end of the attached spinal nerve is measured using commercially available microscopic image analysis software. Lengths of all neurites are quantitated and compared to those present in vehicle-treated control DRGs. Compounds of this invention elicit a significant increase in the number and/or average length of neurites as compared to vehicle-treated control preparations.

### Example 51

Neuroprotection Assay in Spinal Cord Slice Preparations: All cultures were derived from postnatal day 8 (P8) Sprague-Dawley rat lumbar spinal cord slices of 325 micron thickness, prepared using a commercially available Mcllwain tissue chopper. Each experiment consisted of two 6-well plates with 5 slices from 4 different animals per well. Media changes were performed every 3 to 4 days. Cultures were treated with THA [L(−)-threo-3-hydroxyaspartic acid; Tocris Cookson Inc., Ballwin, Mo.] at 200 μM+compound (10 μM) after one week in culture. The control was an untreated sample with 0.1% DMSO as vehicle. The THA control was a THA treated sample with 0.1% DMSO as vehicle. Two wells were used per condition. One media change with new THA and compounds was performed. The experiment was stopped 6 to 8 days following drug treatment (13-15 total days in vitro, DIV) as dictated by visual assessment of lesion, by fixation with 4% paraformaldehyde/0.1 M phosphate buffer for 30 minutes. Slices were permeabilized with 100% cold methanol for 10 minutes and transferred to staining wells. The slices were blocked with 10% HS/TBS. Primary antibody incubation was overnight at 4° C. with SMI-32 antibody 1:5000 in 2% HS/TBS. SMI-32 was specific towards unphosphorylated H neurofilament subunit. Vectastain ABC Elite Kit with rat absorbed anti-mouse secondary antibody was used with 3,3-diarninobenzidine as a chromogen to stain the slices. The slices were mounted onto a slide and a coverslip was sealed with DPX mounting solution. Quantification of surviving neurons was performed on a Zeiss Axiovert microscope. Neuronal survival was determined by observing an intact neuronal cell body with processes located ventrally of the central canal in each hemisphere. This correlated to laminae VII, VIII and IX. Each hemisphere was counted individually. The statistics were performed with StatView™ software on a minimum of three different experiments per condition and significance was determined as compared to THA control. The percent of protection was determined from the average number of living neurons by the following equation: (drug treatment condition−THA control)/(Untreated control−THA control). Untreated control cultures displayed an average of 26.4±4.2 (mean±standard error) SMI-32 immunoreactive neurons per ventral hemisphere of the spinal cord slices at the end of the culturing interval, while THA-treated control cultures displayed a significantly reduced number of 15.97±2.04 cells. Addition of Compound 15a to THA-treated cultures caused a complete protection from THA-induced cell death (26.5±2.7 cells/ventral hemisphere). Other compounds of this invention are expected to elicit a significant increase in the numbers of surviving neurons as compared to control cultures.

### Example 52

Inhibition of Mitochondrial Permeability Transition in a Spectrophotometric Large Amplitude Mitochondrial Swelling Assay: Fresh rat liver mitochondria are prepared from male Sprague-Dawley rats as described by Broekemeier, et al., J. Biol. Chem. 260:105-113 (1985). Incubations are conducted at room temperature in an assay buffer containing 10 mM sodium succinate, 3 mM Hepes (pH 7.4), 5 μM rotenone, 0.5 μg/ml oligomycin, 10 μM CaCl₂, and mannitol/sucrose at a ratio of 3:1 to yield an osmotic strength of 300 mosmoles. Five μl of the isolated mitochondria preparation and 5 μl of compound or vehicle solution are added at various concentrations and optical density (OD) is read at 540 nm for one minute to obtain a baseline reading. Ten μl of ruthenium red solution is added to yield a final concentration of 1 μM, and OD₅₄₀ is monitored for an additional minute. Twenty-five μl of fluoro-carbonyl cyanide solution is added to yield a final concentration of 4 μM, and OD₅₄₀ is monitored for an additional 4-5 minutes. Mitochondrial permeability transition is manifested as a progressive drop in net absorbance as the mitochondria swell. The ability of the compounds of the invention to inhibit mitochondrial permeability transition and swelling can be expressed as IC₅₀ values. Compounds of this invention significantly inhibit the progressive drop of net absorbance at OD₅₄₀, and inhibit the mitochondrial permeability transition in a dose-dependent manner.

### Example 53

In Vivo Reinnervation of the Denervated Striatum by Nigrostriatal Dopaminergic Fibers: The MPTP-lesioned mouse model of Parkinson's disease was utilized to demonstrate in vivo efficacy of the compounds of this invention. MPTP (N-methyl-4-phenyl-1,2,3,6-tetrahydropyridine) is a systemically available neurotoxin specific to nigrostriatal dopaminergic neurons, i.e. to the cells that degenerate in human Parkinson's disease. Administration of MPTP to mice leads to a selective partial destruction of the mesotelencephalic dopaminergic projection, and to a loss of dopamine and dopaminergic fibres in the corpus striatum, which is the main forebrain target of midbrain dopaminergic neurons.

Young adult male CD1 albino mice (Harlan—Sprague Dawley; 22-25 g) were dosed i.p. with the dopamine cell-specific neurotoxin N-methyl-4-phenyl-1,2,3,6-tetrahydropyridine (MPTP hydrochloride, calculated as 34 mg/kg free base), dissolved in saline at a concentration of 3.4 mg/ml free base once daily on days one to five.

Experimental compounds were administered once daily on days 1-5 (10 mg/kg in Intralipid vehicle, s.c.), one hour prior to MPTP-administration.

On day seven, animals were perfused transcardially with 10% neutral buffered formalin. Sagittal sections of striatal tissue were cut at 20 μm thickness on a freezing microtome and processed for free-floating tyrosine hydroxylase immunocytochemistry using a polyclonal TH antibody (Pel Freeze, 1:2500 under refrigeration for 4 nights), further processed using the avidin:biotin peroxidase method (Vector Elite kit), and visualized with Diamino benzidine (DAB-HCl, Polysciences).

Blinded analysis of TH fiber density in the central striatum was performed at 630× magnification. For each mouse striatum, five representative 100 μm×100 μm fields in the central striatum were photographed using a digital video camera. The percentage of sample field covered by TH positive processes and terminals was calculated using an image analysis program (“Simple,” Compix Inc., Pittsburgh, Pa.). The mean striatal innervation density was calculated for each group. The magnitude of striatal deafferentation due to the MPTP lesion was assessed by dividing the observed striatal innervation values obtained in MPTP/vehicle treated cases by the mean striatal innervation density in the Vehicle/Vehicle group and expressed as % loss. The relative efficacy of the compounds of this invention was expressed as % protection of striatal innervation density, i.e., the degree to which the density of TH positive fibres in the striatum of lesioned/compound-treated animals exceeded the loss observed in lesioned-alone animals.

Experimental animals treated with compound 8a of this invention according to the above protocol displayed a 37,2% protection of striatal tyrosine hydroxylase-immunorecative fibres. Treatment with compound 15a resulted in a 72.4% protection of striatal tyrosine hydroxylase-immunoreactive fibres relative to control animals. Administration of other compounds of this invention is expected to lead to a significant protection of striatal dopaminergic innervation density from neurotoxin-induced lesion.

### Example 54

In Vivo Protective Effects in an Animal Model of Cerebral Stroke: Male Sprague Dawley rats, weighing 260-290 g, are used in determining the protective effects of the compounds of the invention against ischemia-induced brain damage. The compounds are dissolved in 50 mM Hepes buffered saline or another physiologically acceptable vehicle, and the pH is adjusted to 7.4 before administration. The compound is administered intravenously 60 min. following experimental medial cerebral artery occlusion (MCAO) at a bolus dose of, e.g., 100 mg/kg immediately followed by an infusion dose of 20 mg/kg/hr for 4 hours. MCAO surgery: The intraluminal filament model of transient MCAO is well established in the art [see, e.g., Lu, et al., Eur. J. Pharmacol. 408: 233-239 (2000)]. Briefly, under 1.5% halothane anesthesia, the rat common carotid artery is exposed at the level of external and internal carotid artery bifurcation. The external carotid artery (ECA) and its branches are cauterized and cut. A piece of 3-0 monofilament nylon suture with a blunted tip is introduced into the internal carotid artery (ICA) via the proximal end of the ECA stump. The suture is advanced through the carotid canal to the origin of the MCA where it blocks the blood flow to its entire territory. At the end of the 2 hour occlusion period, the rat is re-anesthetized and the suture is carefully pulled back to the ECA stump to allow reperfusion. During the surgery, the animal's body temperature is maintained at 37.0° C. via a heating blanket. The experimental animals are sacrificed following 22 hr of reperfusion. The brains are removed and cut into seven 2-mm thick coronal slices, stained with 1% 2,3,5-triphenytetrazolium chloride (TTC), and subsequently imaged using a computer-assisted digital imaging analysis system. The ischemic injury is quantified based on the volume of the infarct tissue completely lacking TTC staining. The total infarct volume and the infarct volumes of the cortical and subcortical regions of each rat are used for statistical analysis. A one-factor analysis of variance can be used for comparison of treatment effects. The difference between groups is considered statistically significant at p<0.05. Administration of compounds of this invention causes a significant reduction in infarct volume as compared to vehicle-treated animals.

### Example 55

In Vivo Protective Effects in an Animal Model of Myocardial Infarction: The surgical procedures and protocol for inducing experimental myocardial infarction is itself well-established in the art [see, e.g., Kukreja, et al., Mol. Cell. Biochem., 195: 123-131 (1999)]. Briefly, male Sprague-Dawley rats (225-300 g) are anaesthetized with 65 mg/kg sodium pentobarbital i.p;. Following tracheotomy, animals are mechanically ventilated using 35% O₂/65% N₂ at 50 strokes/min. and a stroke volume of 2 ml, and maintained at 37.0° C. using a heating blanket. Electrocardiographic leads are attached to subcutaneous electrodes to monitor either limb leads I, II or III. The right carotid artery is cannulated and connected to a pressure transducer to monitor arterial pressure throughout the experiment, and the right jugular vein is cannulated to allow intravenous administration of compounds of the invention. The compounds are dissolved in 50 mM Hepes buffered saline or another physiologically acceptable vehicle, and the pH is adjusted to 7.4 before administration. The compound is administered intravenously 20 min prior to experimental coronary artery occlusion at a bolus dose of, e.g., 100 mg/kg, immediately followed by an infusion dose of 20 mg/kg/hr for 140 minutes. A left thoracotomy is performed at the fourth intercostal space and the heart exposed. A 5-0 silk suture with a traumatic needle is then passed around the left coronary artery midway between the atrioventricular groove and the apex, and the ends of the suture thread are passed through a piece of vinyl tubing to form a snare. The coronary artery is transiently occluded by tightening and fixing the snare. Myocardial ischemia can be confirmed visually by regional cyanosis of the exposed heart, hypokinetic movement of the heart muscle, or by ST segment elevation/depression or T wave inversion on the electrocardiogram. The snare is released after 30 minutes and reperfusion is visually confirmed by hyperemia over the previously cyanotic area of the heart muscle, and by hemodynamic improvement in blood pressure. Following 90 minutes of reperfusion, the snare is again tightened and approximately 1 ml of Evan's blue dye is injected as a bolus vial the jugular vein catheter. The animals are sacrificed immediately, the hearts are removed, frozen, and cut from apex to base into 6-8 transverse 2 mm-thick slabs. The area at risk is determined by the absence of Evan's blue staining. The slices are then incubated in 1% TTC solution for visualization of viable tissue. The infarct volume and area at risk are quantitated using a commercially available image analysis system. Administration of compounds of this invention causes a significant dose-dependent reduction in infarct volume as compared to animals treated with vehicle alone.

### Example 56

In Vivo Hair Generation: Experimental methods useful in assessing the ability of the present compounds to protect from cancer chemotherapy-induced alopecia are themselves established in the art. [See, e.g., Maurer, et al. Am. J. Pathol. 150(4):1433-41 (1997)]. In addition, a useful experimental model for assessing the ability of compounds to induce hair growth in bald human scalp from subjects with male pattern baldness has been reported. [Sintov, et al., Int. J. Pharm. 194:125-134 (2000)]. Simple procedures for the assessment of hair revitalizing properties of experimental compounds have been disclosed previously by the inventors. See, e.g., U.S. Pat. No. 6,194,440 B1. These and other publications referenced herein can be relied upon to assess the hair growth-promoting and hair loss-retarding properties of compounds of Formulae I or II. The following procedure illustrates:

Mice of the C57B1/6 strain, aged 7-8 weeks, are housed individually. Under light ether anaesthesia, an area of about 2 cm by 2 cm of the lower back/hindquarter region is shaved to remove all existing hair. Care is taken to avoid scrapes, cuts or abrasions of the skin. A pinkish color of the skin confirms that all animals are in the telogen phase of the hair growth cycle. Groups of 10 animals are treated topically with 20% propylene glycol vehicle, or with compounds of the invention at concentrations ranging from 0.1 μM to 100 μM per milliliter vehicle. Compounds are topically administered three times per week, and hair growth is assessed weekly by a blinded observer on a scale of 0 (no growth) to 5 (complete hair growth over shaved area). The compounds of the invention induce the growth of hair in a dose-dependent manner, and significantly shorten the time elapsed until the shaved area is covered by hair as compared to the shaved area of vehicle-treated animals.

As noted above, the specific examples should not be interpreted as a limitation to the scope of the invention. Instead, they are merely exemplary embodiments one skilled in the art would understand from the entire disclosure of this invention. The invention being thus described, it will be obvious that the same may be varied in many ways. Such variations are not to be regarded as a departure from the spirit and scope of the invention and all such modifications are included to be within the scope of the following claims.

### REFERENCES CITED

Each of the references cited below or in the text above can be relied on to make and use any aspect of this invention. While particular uses and references are discussed above, this should not be taken as a limitation on the use of any particular reference. All the references are specifically included into this text by reference, in their entirety.

Apfel, et al., Brain Res. 634: 7-12 (1994);

Ausubel, et al.,eds., Current Protocols in Molecular Biology, John Wiley & Sons, N.Y., (and supplements through December 2000);

Bell, et al., Biochem. Pharmacol. 48:495-503 (1994);

Berriman and Fairlamb, Biochem. J. 334:437-445 (1998);

Broekemeier, et al., J. Biol. Chem. 264: 7826-7830 (1989);

Coligan, et al., eds., Current Protocols in Immunology, John Wiley and Sons, N.Y., (and supplements through December 2000);

Enna, et al., eds., Current Protocols in Pharmacology, John Wiley & Sons, N.Y., (and supplements through December 2000);

Fingl, et al., in The Pharmacological Basis of Therapeutics, Ch. 1, (1975);

Fischer, et al., Biomed. Biochem. Acta 43: 1101-1112 (1984);

Friedman et al., Proc. Natl. Acad. Sci., 90:6815-6819 (1993);

Gash, et al., Nature 380: 252-255 (1996);

Gerlach, et al., Eur. J. Pharmacol.-Mol. Pharmacol. 208: 273-286 (1991);

Gold, et al., Exp. Neurol. 147: 269-278 (1997);

Gold, Mol. Neurobiol. 15: 285-306 (1997);

Griffiths and Halestrap, J. Mol. Cell Cardiol. 25: 1461-1469 (1993);

Hamilton and Steiner, J. Med. Chem. 41: 5119-5143 (1998);

Hamilton, et al., Bioorgan. Med. Chem. Lett. 7: 1785-1790 (1997);

Handschumacher, et al., Science 226:544 (1984);

Harding, et al., Nature, 341:758-760 (1989);

Harrison, et al., Biochem. 29: 3813-3816 (1990);

Hoffer, et al., J. Neural Transm. [Suppl.] 49: 1-10 (1997);

Holt, et al., Bioorg. Med. Chem. Letters, 4: 315-320 (1994);

Hsu, et al., J. Am. Chem. Soc. 112: 6745-6747 (1990);

Iwabuchi, et al., J. Dennatol. Sci. 9: 64-69 (1995);

Jiang, et al., J. Invest. Dermatol., 104 523-525 (1995);

Justice, et al., Biochem. Biophys. Res. Commun. 171: 445-450 (1990);

Khattab, et al., Exp. Parasitol. 90:103-109 (1998);

Kofron, et al., Biochem. 30: 6127-6134 (1991);

Kofron, et al., J. Am. Chem. Soc. 114: 2670-2675 (1992);

Kukreja, et al., Mol. Cell. Biochem., 195: 123-131 (1999);

Kuillertz, et al., Clin. Chem. 44: 502-508 (1998);

Lang, et al., Nature 329: 268-270 (1987);

Leducq, et al., Biochem. J. 336: 501-506 (1998);

Lemasters, et al., Mol. Cell. Biochem. 174: 159-165 (1997);

Li, et al., J. Med. Chem. 43: 1770-9 (2000);

Lu, et al., Eur. J. Pharmacol. 408: 233-239 (2000);

Lyons, et al., Proc. Natl. Acad. Sci. U.S.A. 91:3191-3195 (1994);

Marchetti, et al., J. Exp. Med. 184: 1155-1160 (1996);

Matsumoto, et al., J. Cereb. Blood Flow Metab. 19: 736-41 (1999);

Maurer, et al. Am. J. Pathol. 150(4):1433-41 (1997);

McLauchlan, et al., Parasitology 121:661-70 (2000);

McMahon, et al., Curr. Opin. Neurobiol. 5: 616-624 (1995);

Mucke, et al., Biochem. 31: 7848-7854 (1992);

Palacios, J. Immunol. 128:337 (1982);

Paus, et al., Am. J. Pathol. 144: 719-34 (1994);

Perkins, et al., Antimicrob. Agents Chemother. 42: 843-848 (1998)

Remington's Pharmaceutical Sciences, Mack Publishing Co., Easton, Pa., 18^{th} edition (1990);

Schonbrunner, et al., J. Biol. Chem. 266: 3630-3635 (1991);

Sintov, et al., Int. J. Pharm. 194:125-134 (2000);

Snyder, Nat. Med. 1:32-37 (1995);

Steiner, et al., Proc. Natl. Acad. Sci. U.S.A. 94: 2019-2024 (1997);

Streblow, et al., Virology 245: 197-202 (1998);

Wang, et al., J. Pharnacol. Exp. Therap. 282: 1084-1093 (1997);

Yamamoto, et al., J. Invest. Dermatol. 102 (1994) 160-164;

Yoo, et al., J. Mol. Biol., 269: 780-795 (1997);

Zahner and Schultheiss, J. Helminthol. 61:282-90 (1987);

Zamzami, et al., FEBS Lett. 384: 53-57 (1996).

We claim:

1. A compound of the following formula: 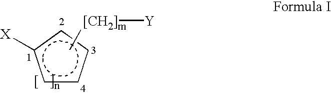
and pharmaceutically acceptable derivatives thereof;
wherein n is 1 or 2, forming a central 5-6 membered carbocyclic ring which is optionally saturated, partially saturated, or aromatic;
m is 0-3;
the substituent —[CH₂]ₘ—Y is attached to said central carbocyclic ring at position 2, 3, or 4;
X and Y are the same or different, and may independently be: 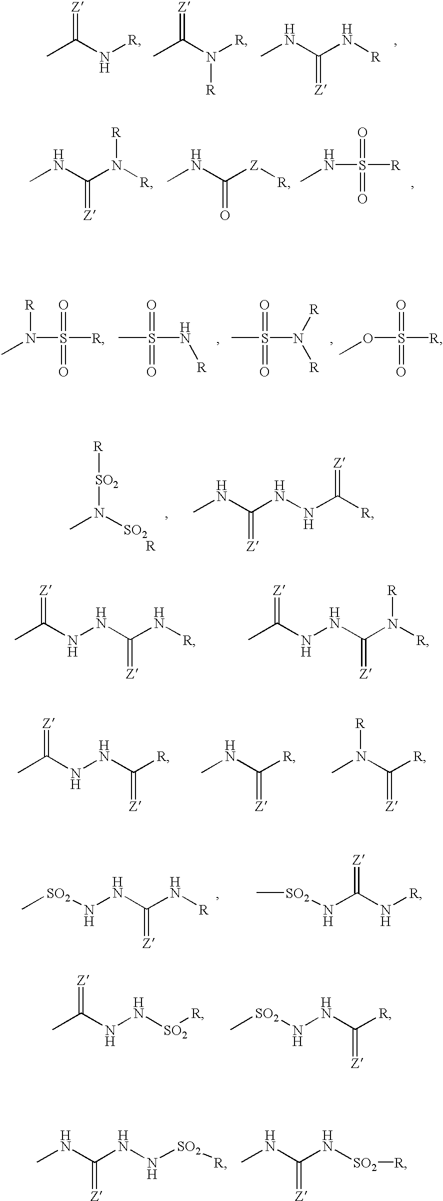
or a combination thereof,
or C₁-C₆ straight or branched chain alkyl, alkenyl, or alkynyl; said alkyl, alkenyl or alkynyl being substituted at one or several positions with Q, and optionally substituted at one or several positions by hydroxyl, mercaptyl, or carbonyl oxygen;
and where Y may further be: Q, 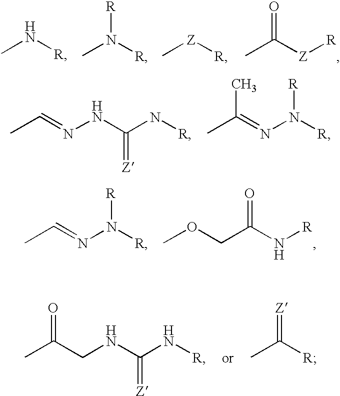
wherein Z′ is O, S, N(CN), CH(NO₂), or N(NO₂);
Z is O or S; and
R may independently be:
Q,
or C₁-C₆ straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;
and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; hydroxyl; mercaptyl; nitro; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state;
provided that:
when R is Q, or Q-substituted C₁-C₆ alkyl or alkenyl, or Q-substituted C₁-C₆ alkyl or alkenyl which is additionally substituted with one or more hydroxyl- or oxo-groups, and
n is 2, and
m is 0, and
Y is attached to said central carbocyclic ring at position 3;
then X and Y are not both 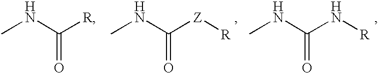
or a combination thereof;
and further provided that:
when R is Q, or Q-substituted C₁-C₆ alkyl or alkenyl, or Q-substituted C₁-C₆ alkyl or alkenyl which is additionally substituted with one or more hydroxyl- or oxo-groups, and
n is 2, and
m is 0, and
Y is attached to said central carbocyclic ring at position 3, and said carbocyclic ring is aromatic;
then X and Y are not both: 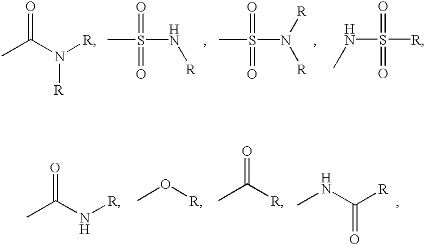
or a combination thereof.

2. A compound of the following formula: 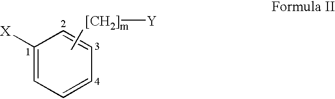
and pharmaceutically acceptable derivatives thereof;
wherein m is 0-3;
the substituent —[CH₂]ₘ—Y is attached at position 2, or 3;
X and Y are the same or different, and may independently be: 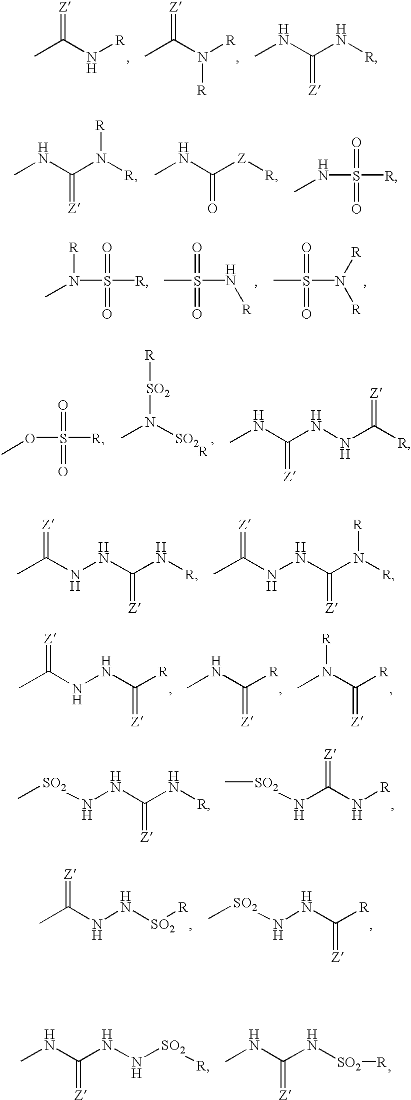
or a combination thereof,
or C₁-C₆ straight or branched chain alkyl, alkenyl, or alkynyl; said alkyl, alkenyl or alkynyl being substituted at one or several positions with Q, and optionally substituted at one or several positions by hydroxyl, mercaptyl, or carbonyl oxygen;
and where Y may further be: Q, 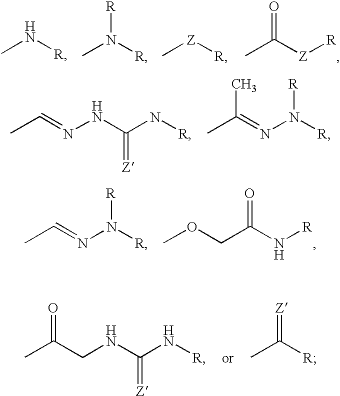
wherein Z′ is O, S, N(CN), CH(NO₂), or N(NO₂);
Z is O or S; and
R may independently be:
Q,
or C₁-C₆ straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;
and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; mercaptyl; nitro; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state;
provided that:
when R is Q, or Q-substituted C₁-C₆ alkyl or alkenyl, or Q-substituted C₁-C₆ alkyl or alkenyl which is additionally substituted with one or more hydroxyl- or oxo-groups, and
m is 0, and
Y is attached at position 3;
then X and Y are not both 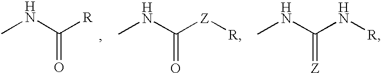
or a combination thereof;
and further provided that:
when R is Q, or Q-substituted C₁-C₆ alkyl or alkenyl, or Q-substituted C₁-C₆ alkyl or alkenyl which is additionally substituted with one or more hydroxyl- or oxo-groups, and
m is 0, and
Y is attached at position 3,
then X and Y are not both: 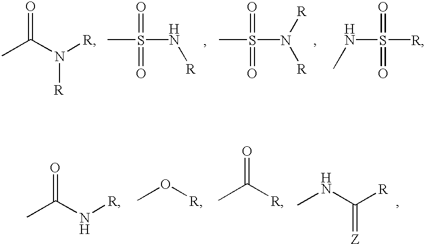
or a combination thereof.

3. A compound of the following formula: 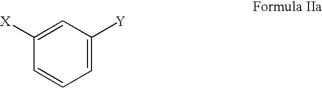
and pharmaceutically acceptable derivatives thereof;
where X and Y are the same or different, and may independently be: 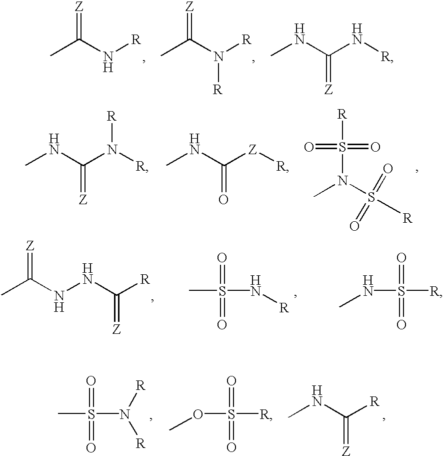
or a combination thereof,
or C1-C6 straight or branched chain lower alkyl, alkenyl, or alkynyl which is substituted at one or several positions with Q, and which further may optionally be substituted at one or several positions by hydroxyl, mercaptyl, or carbonyl oxygen;
and where Y may further be: Q, 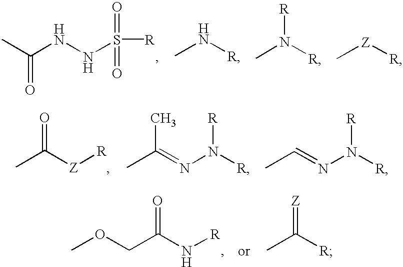
wherein Z is O or S, and
R may independently be:
Q,
or C1-C6 straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;
and wherein Q is a mono-, bi- or tricyclic carbo- or heterocyclic ring which is optionally saturated, partially saturated, or aromatic, and which may optionally be substituted in one or several positions with halo, hydroxyl, mercaptyl, nitro, cyano, trifluoromethyl, C1-C6 straight or branched chain alkyl or -alkenyl, C1-C4 alkoxy or -alkenyloxy, phenoxy, benzyloxy, amino, or acetyl, and wherein the individual ring sizes are 5-6 members, and wherein each heterocyclic ring contains 1-6 heteroatoms selected from the group consisting of O, N, S, or a combination thereof;
provided that:
when R is Q, or Q-substituted C1-C6 straight or branched chain alkyl or alkenyl, or Q-substituted C1-C6 straight or branched chain alkyl or alkenyl which is additionally substituted with one or more hydroxyl- or oxo-groups,
then X and Y are not both 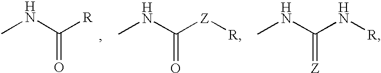
or a combination thereof;
and further provided that:
when R is Q, or Q-substituted C1-C6 straight or branched chain alkyl or alkenyl, or Q-substituted C1-C6 straight or branched chain alkyl or alkenyl which is additionally substituted with one or more hydroxyl- or oxo-groups,
then X and Y are not both: 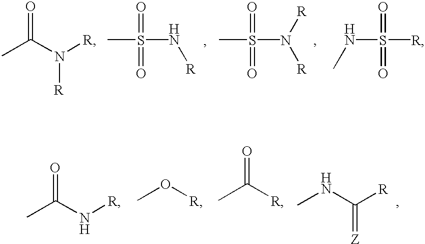
or a combination thereof.

4. A compound of the following formula: 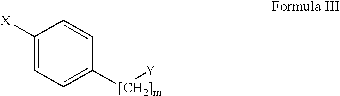
and pharmaceutically acceptable derivatives thereof;
wherein m is 0-3;
X and Y are the same or different, and may independently be: 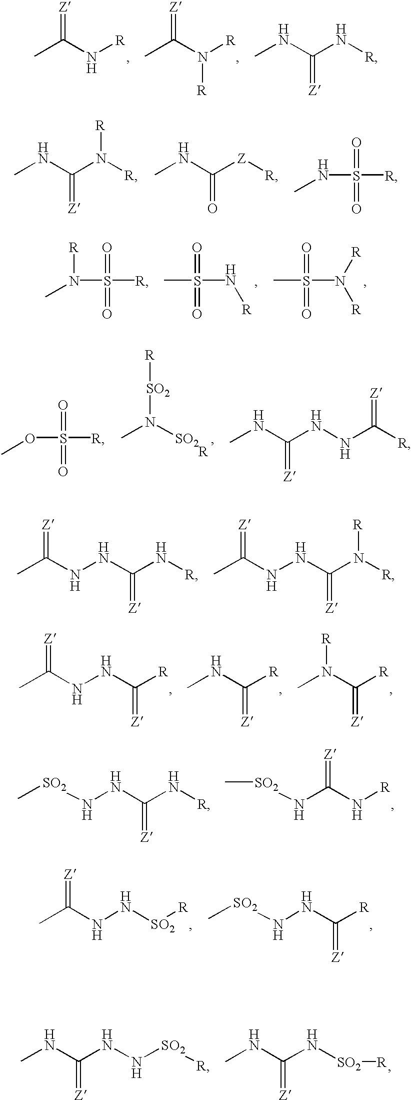
or a combination thereof,
or C₁-C₆ straight or branched chain alkyl, alkenyl, or alkynyl; said alkyl, akenyl or alkynyl being substituted at one or several positions with Q, and optionally substituted at one or several positions by hydroxyl, mercaptyl, or carbonyl oxygen;
and where Y may further be: 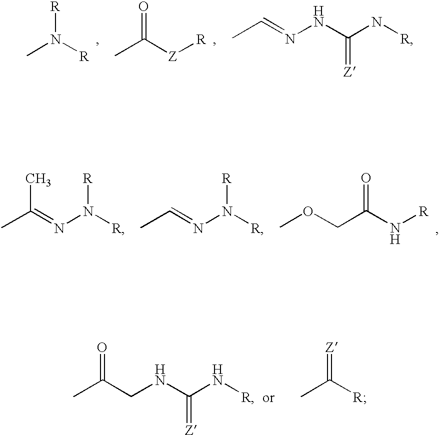
wherein Z′ is O, S, N(CN), CH(NO₂), or N(NO₂);
Z is O or S; and
R may independently be:
Q,
or C₁-C₆ straight or branched chain lower alkyl alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with N, NH, S, SO, or SO₂;
and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; hydroxyl; mercaptyl; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state.

5. A compound of the following formula: 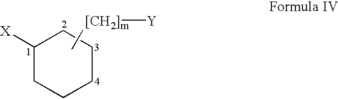
and pharmaceutically acceptable derivatives thereof;
wherein Y is attached at position 2, 3, or 4;
m is 0-3;
the substituent —[CH₂]ₘ—Y is attached at position 2, 3, or 4;
X and Y are the same or different, and may independently be: 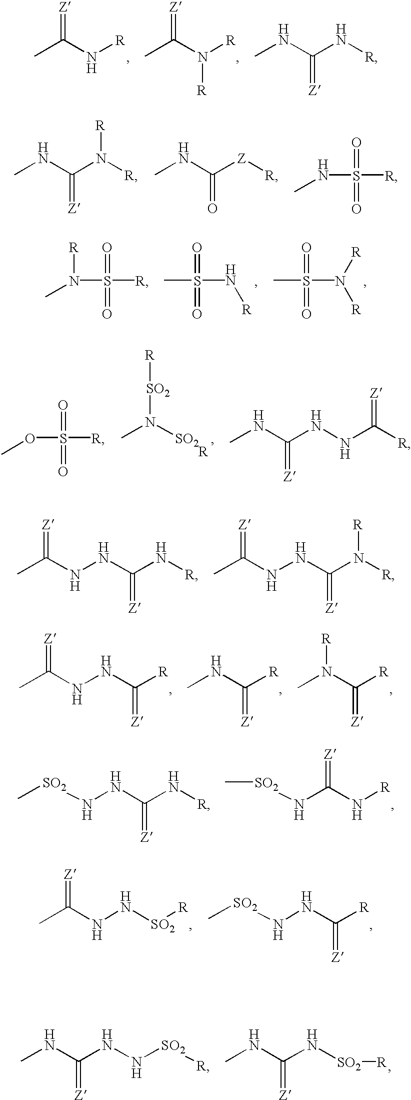
or a combination thereof,
or C₁-C₆ straight or branched chain alkyl, alkenyl, or alkynyl; said alkyl, alkenyl or alkynyl being substituted at one or several positions with Q, and optionally substituted at one or several positions by hydroxyl, mercaptyl, or carbonyl oxygen;
and where Y may further be: Q, 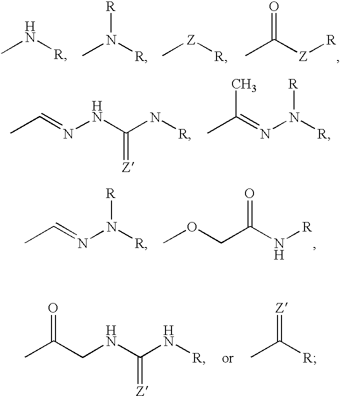
wherein Z′ is O, S, N(CN), CH(NO₂), or N(NO₂);
Z is O or S; and
R may independently be:
Q,
or C₁-C₆ straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;
and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; hydroxyl; mercaptyl; nitro; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state;
provided that:
when R is Q, or Q-substituted C₁-C₆ alkyl or alkenyl, or Q-substituted C₁-C₆ alkyl or alkenyl which is additionally substituted with one or more hydroxyl- or oxo-groups, and
m is 0, and
Y is attached at position 3;
then X and Y are not both 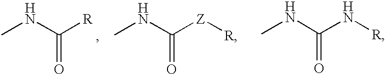
or a combination thereof.

6. A compound of the following formula: 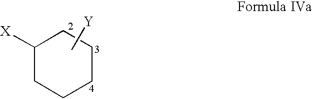
and pharmaceutically acceptable derivatives thereof;
wherein Y is attached at position 2, 3, or 4;
where X and Y are the same or different, and may independently be: 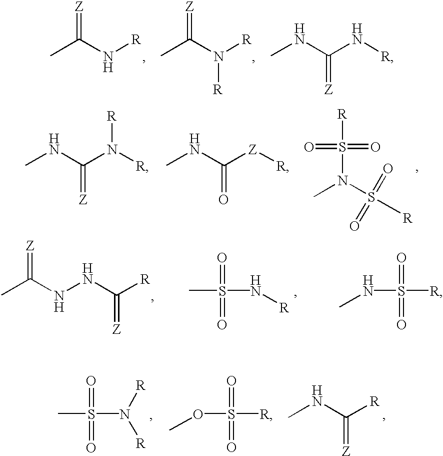
or a combination thereof,
or C1-C6 straight or branched chain lower alkyl, alkenyl, or alkynyl which is substituted at one or several positions with Q, and which further may optionally be substituted at one or several positions by hydroxyl, mercaptyl, or carbonyl oxygen;
and where Y may further be: Q, 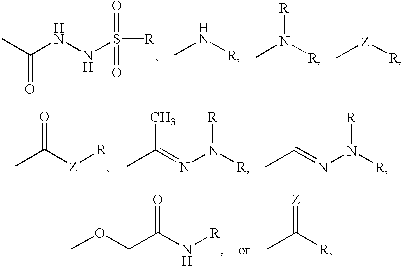
wherein Z is O or S, and
R may independently be:
Q,
or C1-C6 straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;
and wherein Q is a mono-, bi- or tricyclic carbo- or heterocyclic ring which is optionally saturated, partially saturated, or aromatic, and which may optionally be substituted in one or several positions with halo, hydroxyl, mercaptyl, nitro, cyano, trifluoromethyl, C1-C6 straight or branched chain alkyl or -alkenyl, C1-C4 alkoxy or -alkenyloxy, phenoxy, benzyloxy, amino, or acetyl, and wherein the individual ring sizes are 5-6 members, and wherein each heterocyclic ring contains 1-6 heteroatoms selected from the group consisting of O, N, S, or a combination thereof;
provided that:
when R is Q, or Q-substituted C1-C6 straight or branched chain alkyl or alkenyl, or Q-substituted C1-C6 straight or branched chain alkyl or alkenyl which is additionally substituted with one or more hydroxyl- or oxo-groups,
and Y is attached at position 3,
then X and Y are not both 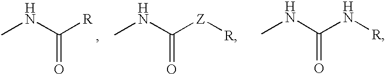
or a combination thereof.

7. A compound of the following formula: 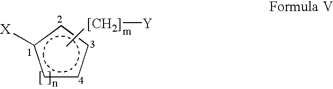
and pharmaceutically acceptable derivatives thereof;
wherein n is 1, forming a central 5-membered carbocyclic ring which is saturated or partially saturated;
m is 0-3;
the substituent —[CH₂]ₘ—Y is attached to said central carbocyclic ring at position 2, 3, or 4;
X and Y are the same or different, and may independently be: 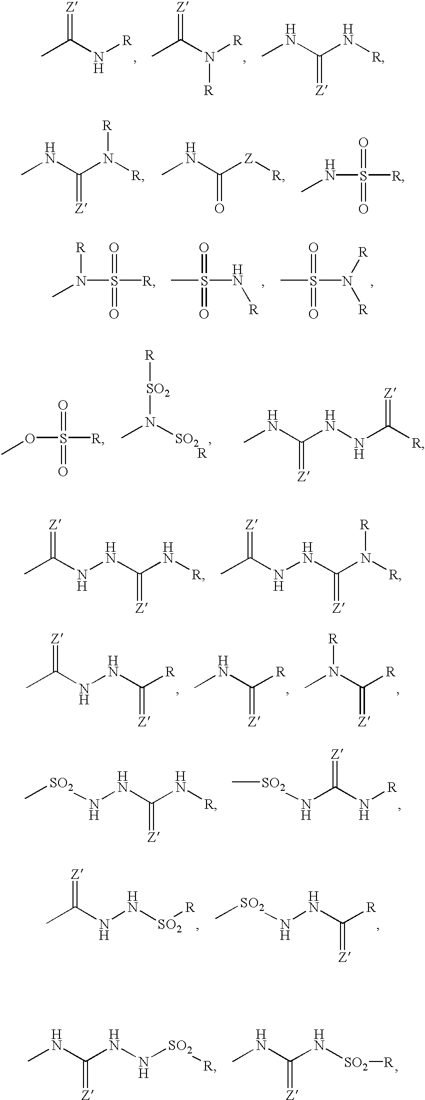
or a combination thereof,
or C₁-C₆ straight or branched chain alkyl, alkenyl, or alkynyl; said alkyl, alkenyl or alkynyl being substituted at one or several positions with Q, and optionally substituted at one or several positions by hydroxyl, mercaptyl, or carbonyl oxygen;
and where Y may further be: Q, 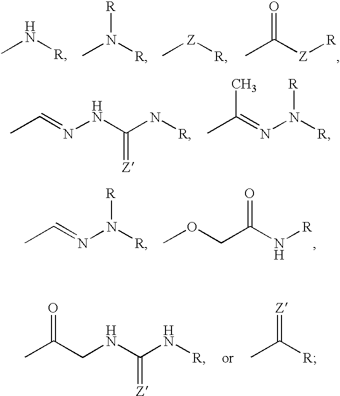
wherein Z′ is O, S. N(CN), CH(NO₂), or N(NO₂);
Z is O or S; and
R may independently be:
Q,
or C₁-C₆ straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;
and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; hydroxyl; mercaptyl; nitro; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state;

8. A compound of the following formula: 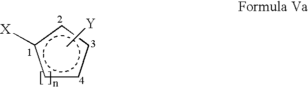
and pharmaceutically acceptable derivatives thereof;
where n is 1, forming a central 5-membered carbocyclic ring which is saturated or partially saturated;
Y is attached to said central carbocyclic ring at position 2, 3, or 4;
X and Y are the same or different, and may independently be: 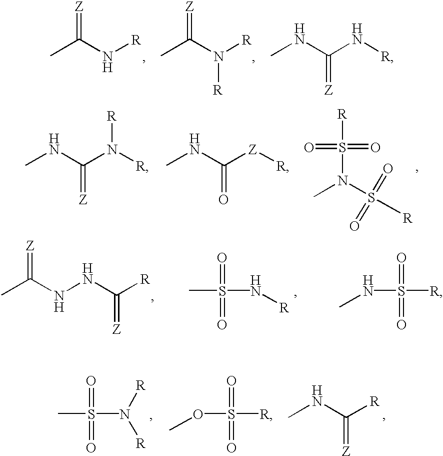
or a combination thereof,
or C1-C6 straight or branched chain lower alkyl, alkenyl, or alkynyl which is substituted at one or several positions with Q, and which further may optionally be substituted at one or several positions by hydroxyl, mercaptyl, or carbonyl oxygen;
and where Y may further be: Q, 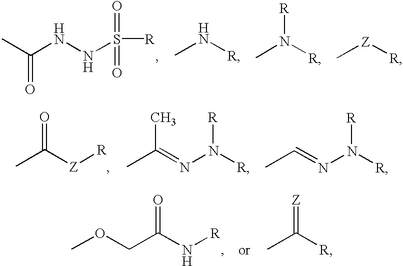
wherein Z is O or S, and
R may independently be:
Q,
or C1-C6 straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;
and wherein Q is a mono-, bi- or tricyclic carbo- or heterocyclic ring which is optionally saturated, partially saturated, or aromatic, and which may optionally be substituted in one or several positions with halo, hydroxyl, mercaptyl, nitro, cyano, trifluoromethyl, C1-C6 straight or branched chain alkyl or -alkenyl, C1-C4 alkoxy or -alkenyloxy, phenoxy, benzyloxy, amino, or acetyl, and wherein the individual ring sizes are 5-6 members, and wherein each heterocyclic ring contains 1-6 heteroatoms selected from the group consisting of O, N, S, or a combination thereof.

9. A compound of the following formula: 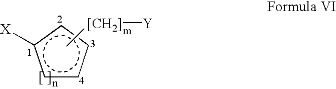
and pharmaceutically acceptable derivatives thereof;
wherein n is 2, forming a central 6 membered carbocyclic ring which is saturated or partially saturated;
m is 0-3;
the substituent —[CH₂]ₘ—Y is attached to said central carbocyclic ring at position 2, 3, or 4;
X and Y are the same or different, and may independently be: 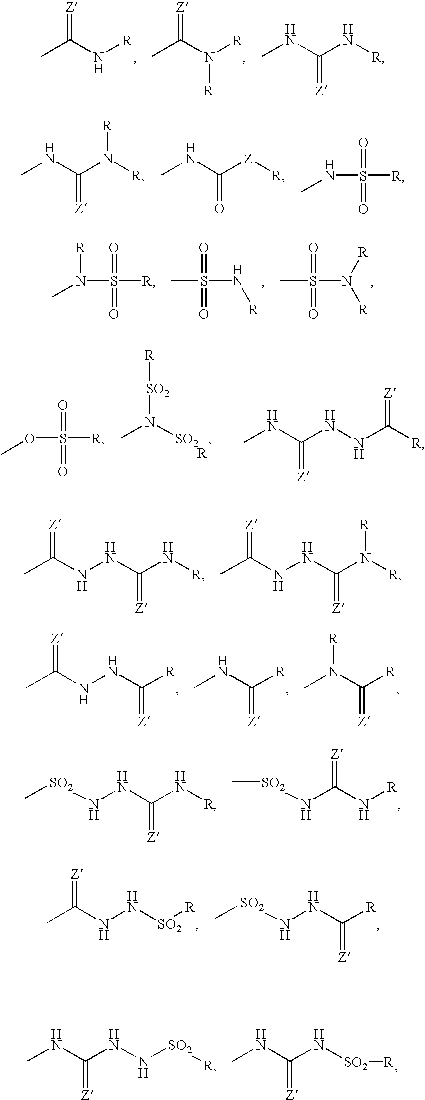
or a combination thereof,
or C₁-C₆ straight or branched chain alkyl, alkenyl, or alkynyl; said alkyl, alkenyl or alkynyl being substituted at one or several positions with Q, and optionally substituted at one or several positions by hydroxyl, mercaptyl, or carbonyl oxygen;
and where Y may further be: Q, 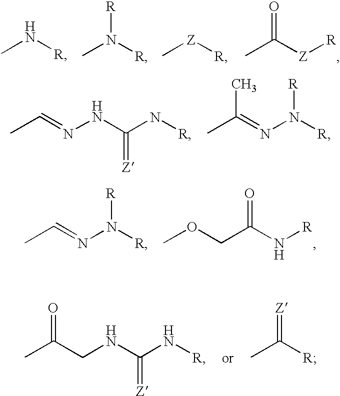
wherein Z′ is O, S, N(CN), CH(NO₂), or N(NO₂);
Z is O or S; and
R may independently be:
Q,
or C₁-C₆ straight or branched chain lower alkyl, alkenyl or alkynyl which is substituted at one or several positions with Q, and which further may optionally be substituted in one or several positions by hydroxyl, mercaptyl, or carbonyl oxygen, and wherein one or more of the carbon atoms are optionally replaced with O, N, NH, S, SO, or SO₂;
and wherein Q, which is optionally saturated, partially saturated, or aromatic, is a mono-, bi-, or tricyclic, carbo- or heterocyclic ring, which is optionally and independently substituted in one or several positions with a substituent selected from the goup consisting of halo; hydroxyl; mercaptyl; nitro; trifluoromethyl; aminocarbonyl; arylaminocarbonyl which is optionally halogenated and optionally substituted with trifluoromethyl or cyano; arylamino which is optionally halogenated; C₁-C₄ alkylsulfonyl; C₁-C₄ alklylthio; C₁-C₄ alkanoyl; oxo; cyano; carboxy; C₁-C₆ alkyl or alkenyl; C₁-C₄ alkoxy; C₁-C₅ alkoxycarbonyl; C₁-C₄ alkenyloxy; phenoxy; phenyl; cyanophenyl; benzyloxy; benzyl; amino; C₁-C₄ alkylamino; di-(C₁-C₄) alkylamino; C₁-C₄ alkylcarbamoyl; and di(C₁-C₄)alkylcarbamoyl, and wherein the individual ring sizes are 5-6 members, and wherein each heterocyclic ring contains 1-6 heteroatoms independently selected from the group consisting of O, N, and S in any chemically stable order and oxidation state;
provided that:
when R is Q, or Q-substituted C₁-C₆ alkyl or alkenyl, or Q-substituted C₁-C₆ alkyl or alkenyl which is additionally substituted with one or more hydroxyl- or oxo-groups, and
m is 0, and
Y is attached to said central carbocyclic ring at position 3;
then X and Y are not both 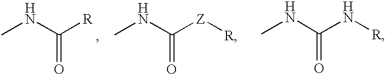
or a combination thereof;
and further provided that:
when R is Q, or methyl monosubstituted with Q, and
m is 0, and
Y is attached to said 6-membered carbocyclic ring at position 2, and said carbocyclic ring is partially saturated,
then X and Y are not both —(CO)—NH—R.

10. The compound according to any one of claims 1, 2, 4, 7, or 9, wherein X and Y are independently selected from the group consisting of

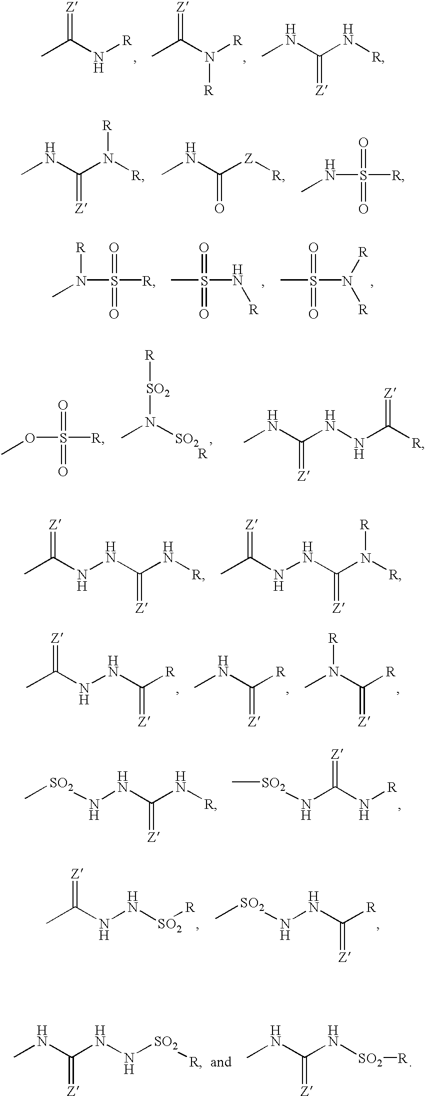

11. The compound according to claim 10, wherein R in one of X or Y is Q.

12. The compound according to claim 10, wherein R in one of X or Y is Q-substituted C₁-C₆ straight or branched chain lower alkyl, alkenyl or alkynyl, which is optionally substituted in one or several positions by hydroxyl, mercaptyl, or carbonyl oxygen.

13. The compound according to any one of claims 1, 2, 5, 7, or 9, wherein Y is selected from the group consisting of

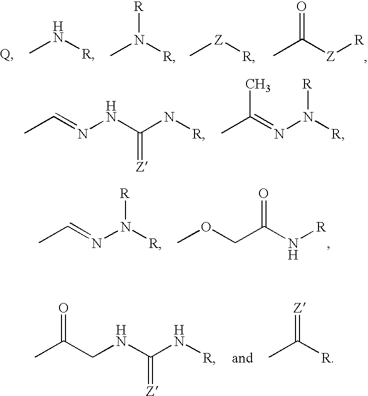

14. The compound according to any one of claims 3, 6, or 8, wherein X and Y are independently selected from the group consisting of:

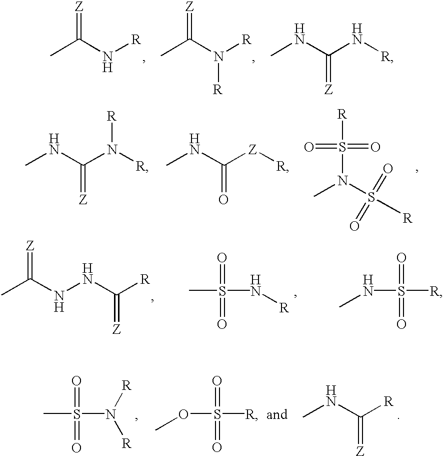

15. The compound of claim 14, wherein R in one of X or Y is Q.

16. The compound of claim 14, wherein R in one of X or Y is Q-substituted C₁-C₆ straight or branched chain lower alkyl, alkenyl or alkynyl, which is optionally substituted in one or several positions by hydroxyl, mercaptyl, or carbonyl oxygen.

17. The compound according to any one of claims 3, 6, or 8, wherein Y is selected from the group consisting of Q,

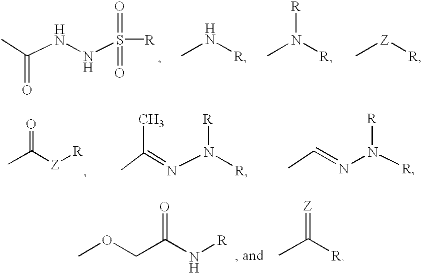

18. The compound according to claim 5, wherein X and the substituent —[CH₂]ₘ—Y are attached in a cis-configuration.

19. The compound according to claim 5 wherein X and the substituent —[CH₂]ₘ—Y are attached in a trans-configuration.

20. The compound according to claim 6 wherein X and Y are attached in a cis-configuration.

21. The compound according to claim 6, wherein X and Y are attached in a trans-configuration.

22. The compound according to claim 7, wherein X and the substituent —[CH₂]ₘ—Y are attached to said central carbocyclic ring in a cis-configuration.

23. The compound according to claim 7, wherein X and the substituent —[CH₂]ₘ—Y are attached to said central carbocyclic ring in a trans-configuration.

24. The compound according to claim 8, wherein X and Y are attached to said central carbocyclic ring in a cis-configuration.

25. The compound according to claim 8, wherein X and Y are attached to said central carbocyclic ring in a trans-configuration.

26. The compound according to claim 9, wherein X and the substituent —[CH₂]ₘ—Y are attached to said central carbocyclic ring in a cis-configuration.

27. The compound according to claim 9, wherein X and the substituent —[CH₂]ₘ—Y are attached to said central carbocyclic ring in a cis-configuration.

28. A compound of the following formula: 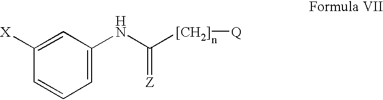
and pharmaceutically acceptable derivatives thereof;
wherein Z is O or S;
n is 2-6;
X is selected from the group consisting of 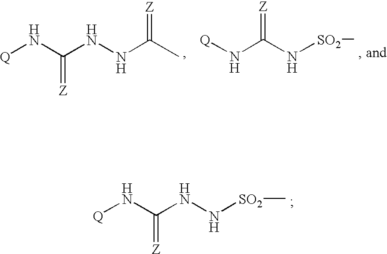
and Q is a 5-6-membered carbo- or heterocyclic ring, which is optionally saturated, partially saturated, or aromatic, and wherein each of one or several heteroatoms, if present, is independently selected from the group consisting of O, N, and S, and wherein Q is optionally substituted at one or several positions with halo or trifluoromethyl.

29. A compound selected from the group consisting of:
Compound 1: [(3,5-dichlorophenyl)amino]-N-(3-{[(4-methoxyphenyl)sulfonyl][(4-methylphenyl)sulfonyl]amino}phenyl)formamide;
Compound 2: [(3,5-dichlorophenyl)amino]-N-(3-{bis[(4-methylphenyl)sulfonyl]amino}phenyl)formamide;
Compound 3: (3,5-dichlorophenyl)-N-(3-{[(4methoxyphenyl)sulfonyl][(4-methylphenyl)sulfonyl]amino}phenyl)formamide;
Compound 4: (3-{bis[(3,5-dichlorophenyl)sulfonyl]amino}phenyl)[(4-methoxyphenyl)sulfonyl][(4-methylphenyl)sulfonyl]amine;
Compound 5: bis[(3,5-dichlorophenyl)sulfonyl](3-{[(naphthylamino)thioxomethyl]amino}phenyl)amine;
Compound 6: N-(3-{bis[(3,5-dichlorophenyl)sulfonyl]amino}phenyl)[(2,6-dichlorophenyl)amino]formamide;
Compound 7: N-(3-{bis[(3,5-dichlorophenyl)sulfonyl]amino}phenyl)[(3,5-dichlorophenyl)amino]formamide;
Compound 8: (3,5-dichlorophenyl)-N-{3-[bis(2-naphthylsulfonyl)amino]phenyl}formamide;
Compound 8a: N-(3-{bis[(3,5-dichlorophenyl)sulfonyl]amino}phenyl)(3,5-dichlorophenyl)formamide;
Compound 9: (3-{bis[(3,5-dichlorophenyl)sulfonyl]amino}phenyl)bis(2-naphthylsulfonyl)amine;
Compound 10: (3-{bis[(3,5-dichlorophenyl)sulfonyl]amino}phenyl)bis[(4-methoxyphenyl)sulfonyl]amine;
Compound 11: (naphthylamino)[(2-{[(naphthylamino)thioxomethyl]amino}cyclohexyl)amino]methane-1-thione;
Compound 12: {[3,5-bis(trifluoromethyl)phenyl]amino}({2-[({[3,5-bis(trifluoromethyl)phenyl]amino}thioxomethyl)amino]cyclohexyl}amino)methane-1-thione;
Compound 13: [(4-iodophenyl)amino]{[2-({[(4-iodophenyl)amino]thioxomethyl}amino)cyclohexyl]amino}methane-1-thione;
Compound 14: [(3,4-dichlorophenyl)amino]{[2-({[(3,4-dichlorophenyl)amino]thioxomethylamino)cyclohexyl]amino}methane-1-thione;
Compound 15: [(3,5-dichlorophenyl)amino]{[2-({[(3,5-dichlorophenyl)amino]thioxomethyl}amino)cyclohexyl]amino}methane-1-thione;
Compound 15a: cis-[(3,5-dichlorophenyl)amino]-N-(2-{[(3,5-dichlorophenyl)amino]carbonylamino}cyclohexyl)formamide;
Compound 16: cis-[(3,5-dichlorophenyl)amino]-N-(4-{[(3,5-dichlorophenyl)amino]carbonylamino}cyclohexyl)formamide;
Compounds 17 and 19: N-(3,5-dichlorophenyl)[3-({[(3,5-dichlorophenyl)amino]thioxomethyl}amino)cyclopentyl]formamide;
Compound 18: (1S,3R)-N-(3,5-dichlorophenyl)[4-({[(3,5-dichlorophenyl)amino]thioxomethyl}amino)cyclopent-2-enyl]formamide;
Compound 20: [(3,5-dichlorophenyl)amino]({3-[2,2-bis(4-chlorophenyl)vinyl]phenyl}amino)methane-1-thione;
Compound 21: ({3-[2-aza-2-(diphenylamino)vinyl]phenyl}amino)[(3,5-dichlorophenyl)amino]methane-1-thione;
Compound 22: 3-({[(3,5dichlorophenyl)amino]thioxomethyl}amino)phenyl 2,3,4,5,6-pentafluorobenzenesulfonate;
Compound 23: 1-{3-[3,5-Bis(trifluoromethyl)benzyloxy]phenyl}-5-(3,5-dichlorophenyl)-1,4-dioxo-2,3,5-triazapentane;
Compound 24: N-(3,5-dichlorophenyl)-2-{3-[(3,5-dichlorophenyl)carbonylamino]phenoxy}ethanamide;
Compound 25: 3-[(3,5-dichlorophenyl)carbonylamino]phenyl 2,3,4,5,6-pentafluorobenzenesulfonate;
Compound 26: {[3,5-bis(trifluoromethyl)phenyl]amino}-N-(3-phenoxyphenyl)formamide;
Compound 27: [(3,5-dichlorophenyl)amino]-N-(2-{[(3,5-dichlorophenyl)amino]carbonylamino}phenyl)formamide;
Compound 28: [(3,5-dichlorophenyl)amino]{[2-({[(3,5-dichlorophenyl)amino]thioxomethyl}amino)phenyl]amino}methane-1-thione;
Compound 29: (4-iodophenyl)-N-{2-[(4-iodophenyl)carbonylamino]phenyl}formamide;
Compound 30: 1-{3-[(3-Benzyloxy)phenylcarboxamido]benzoyl}-2-(3,5-dichlorobenzoyl)hydrazine;
Compound 31: 1-{3-[(3-Benzyloxy)phenylcarboxamido]benzoyl}-2-(3,4-dichlorobenzenesulfonyl)hydrazine;
Compound 32: 1-{[1-Aza-2-oxo-7-(3-trifluoromethylphenyl)]heptyl}-3-{[5-(3,4-dichlorophenyl)-1-oxo-2,3,5-triaza-4-thio]pentyl}benzene;
Compound 33: 1-{3-[(5-Phenyl)valeroylamino]benzoyl}-4-(3,4-dichlorophenyl)thiosemicarbazide;
Compound 34A: 1-[3-(6-Phenylpentanoylamino)-benzenesulfonyl]-3-(3,4-dichlorophenyl)thiourea;
Compound 34B: 1-[3-(6-Phenylpentanoylamino)-benzenesulfonyl]-3-(3,4-dichlorophenyl)urea;
Compound 35: 1-[3-(6-Phenylpentanoylamino)-benzenesulfonyl]-4-(3,4-dichlorophenyl)thiosemicarbazide;
Compound 36: L-N-[3-(6-Phenylhexanoylamino)benzoyl]proline 3,4-dichlorobenzamide;
Compound 37: 1-{3-[(7-Phenyl)heptanoylamino]benzoyl}-4-(3,4-dichlorophenyl)thiosemicarbazide:
Compound 38: 1-{[1-Aza-2-oxo-6-(thien-2-yl)]hexyl}-3-{[5-(3,4-dichlorophenyl)-1-oxo-2,3,5-triaza-4-thio]pentyl}benzene;
Compound 39: N-{3-[(1E)-2-aza-2-({[(3,4-dichlorophenyl)amino]thioxomethyl}amino)vinyl]phenyl}-5-phenylpentanamide;
Compound 40A: 5-Phenyl-pentanoic acid {3-[5-(3,4-dichloro-phenylamino)-[1,3,4]thiadiazol-2-yl]-phenyl}-amide;
Compound 40B: 5-Phenyl-pentanoic acid {3-[5-(3,4-dichloro-phenylamino)-[1,3,4]oxadiazol-2-yl]-phenyl}-amide;
Compound 41: 1-[(6-Phenyl-1-aza-2-oxo)hexyl]-3-{[(adamant-1-yl)-1-oxo-2,3,5-triaza-4-thio]pentyl}-benzene;
Compound 42: 1-[(6-Phenyl-1-aza-2-oxo)hexyl]-3-{[5-(3,4-dichlorophenyl)-1,5-diaza-2,4-oxo]pentyl}-benzene;
Compound 43: 1-{4-[(5-Phenyl)pentanoylamino]benzoyl}-4-(3,4-dichlorophenyl) thiosemicarbazide;
Compound 44: N-{3-[3-(3,5-Dichloro-phenyl)-sulfonyl-ureido]-phenyl}-Di(3,5-dichloro-benzenesulfonamide); and
Compound 45: 1-{3-[6-(3-trifluoromethylphenyl)hexanoylamino]-benzenesulfonyl}-3-(3,4-dichlorophenyl)thiourea.

30. A pharmaceutical composition, comprising:
(i.) a compound of Formula I of claim 2; and
(ii.) a pharmaceutically acceptable carrier, diluent, or excipient.

31. A pharmaceutical composition, comprising:
(i.) a compound of Formula IIa of claim 3; and
(ii.) a pharmaceutically acceptable carrier, diluent, or excipient.

32. A pharmaceutical composition, comprising:
(i.) a compound of Formula III of claim 4; and
(ii.) a pharmaceutically acceptable carrier, diluent, or excipient.

33. A pharmaceutical composition, comprising:
(i.) a compound of Formula IV of claim 5; and
(ii.) a pharmaceutically acceptable carrier, diluent, or excipient.

34. A pharmaceutical composition, comprising:
(i.) a compound of Formula IV of claim 6; and
(ii.) a pharmaceutically acceptable carrier, diluent, or excipient.

35. A pharmaceutical composition, comprising:
(i.) a compound of Formula V of claim 7; and
(ii.) a pharmaceutically acceptable carrier, diluent, or excipient.

36. A pharmaceutical composition, comprising:
(i.) a compound of Formula Va of claim 8; and
(ii.) a pharmaceutically acceptable carrier, diluent, or excipient.

37. A pharmaceutical composition, comprising:
(i.) a compound of Formula VI of claim 9; and
(ii.) a pharmaceutically acceptable carrier, diluent, or excipient.

38. A pharmaceutical composition, comprising:
(i.) a compound of Formula VII of claim 28; and
(ii.) a pharmaceutically acceptable carrier, diluent, or excipient.

39. A pharmaceutical composition, comprising:
(i.) a compound of Formula I of claim 1,
(ii.) a pharmaceutically acceptable carrier, diluent, or excipient; and
(iii.) an additional agent selected grom the group consisting of hair growth-promoting agents, hair loss-retarding agents, antibiotic agents, antidandruff agents, anti-inflammatory agents, pediculicides, antipruriginous agents, anaesthetic agents, keratolytic agents, antiseborrhoeic agents, antiacne agents, and hair dyes.

40. The pharmaceutical composition according to any one of claims 30-38, further comprising an additional agent selected from the group consisting of hair growth-promoting agents, hair loss-retarding agents, antibiotic agents, antidandruff agents, anti-inflammatory agents, pediculicides, antipruriginous agents, anaesthetic agents, keratolytic agents, antiseborrhoeic agents, antiacne agents, and hair dyes.

41. A method of using a compound to bind a cyclophilin-type immunophilin protein, comprising contacting the compound with a cyclophilin-type immunophilin, wherein the compound is of Formula I as defined in claim 1.

42. The method of claim 41, wherein contacting the compound with a cyclophilin-type immunophilin occurs in vivo.

43. The method of claim 41, wherein contacting the compound with a cyclophilin-type immunophilin occurs in vitro.

44. The method of claim 42, wherein contacting the compound with a cyclophilin-type immunophilin occurs after administration to an animal.

45. The method of claim 50, wherein the animal is human.

46. The method of claim 43, wherein contacting the compound with a cyclophilin-type immunophilin occurs within a cell.

47. The method of claim 43, wherein contacting the compound with a cyclophilin-type immunophilin occurs in a cell-free preparation.

48. A complex of a compound of Formula I of claim 1, and a cyclophilin-type immunophilin.

49. The complex of claim 48, wherein the cyclophilin-type immunophilin is human.

50. A method of using a compound of Formula II of claim 2, comprising administering a pharmaceutically effective amount of the compound to an animal.

51. The method of claim 50, wherein the animal is diagnosed with, is predisposed to, or is suspected of having a neurological disorder.

52. A method of treating a neurological disorder in a patient, comprising administering to said patient a therapeutically effective amount of a compound of Formula I of claim 1, or of a pharmaceutically acceptable derivative thereof, wherein the neurological disorder is a neurodegenerative disorder; neuropathic disorder; neurovascular disorder; traumatic injury of the brain, spinal cord, or peripheral nervous system; demyelinating disease of the central or peripheral nervous system; metabolic or hereditary metabolic disorder of the central or peripheral nervous system; or toxin-induced- or nutritionally related disorder of the central or peripheral nervous system.

53. The method of claim 52, wherein the neurodegenerative disorder is Parkinson's disease, Alzheimer's disease, amyotrophic lateral sclerosis (ALS), Huntington's disease, cerebellar ataxia, or multisystem atrophy.

54. The method of claim 52, wherein the demyelinating disease is multiple sclerosis, Guillain-Barre syndrome, or chronic inflammatory demyelinating polyradiculoneuropathy.

55. The method of claim 52, wherein the neurovascular disorder is global cerebral ischemia, spinal cord ischemia, ischemic stroke, cardiogenic cerebral embolism, hemorrhagic stroke, lacunar infarction, or a multiple infarct syndrome.

56. The method of claim 52, wherein the traumatic injury of the central or peripheral nervous system is concussion injury; contusion injury; diffuse axonal injury; edema; hematoma associated with craniocerebral or spinal trauma; axonal or nerve sheath damage associated with laceration, compression, stretch, or avulsion of peripheral nerves or plexi; or neural tissue damage caused during surgery.

57. The method of claim 56 wherein the surgery is prostate surgery, and the neural tissue damage is to the major pelvic ganglion or to the cavernous nerve.

58. The method of claim 52, wherein the neuropathic disorder is diabetic neuropathy, uremic neuropathy, neuropathy related to drug therapy, or neuropathy associated with viral infection.

59. The method of claim 52, wherein the metabolic disorder is status epilepticus, hypoglycemic coma, or Wilson's disease.

60. A method of preventing a neurological disorder, comprising administering to an animal a pharmaceutically effective amount of a compound of Formula I of claim 1, or of a pharmaceutically acceptable derivative thereof.

61. A method of stimulating hair growth, preventing hair loss, or retarding hair loss in a mammal, comprising administering to said mammal an effective amount of a compound of Formula I of claim 1, or of a pharmaceutically acceptable derivative thereof.

62. The method of claim 61, wherein said mammal is undergoing therapy with a cancer chemotherapeutic agent.

63. The method of claim 62, wherein said cancer chemotherapeutic agent is cisplatin, carboplatin, cyclophosphamide, dactinomycin, etoposide, hexamethamelamine, ifosfamide, taxol, vincristine, bleomycin, or 5-fluorouracil.

64. The method of claim 61, wherein said mammal is undergoing radiation therapy.

65. The method of claim 61, wherein said mammal is suffering from alopecia areata, androgenetic alopecia/male pattern baldness, anagen effluvium, trichotillomania, traction alopecia, or telogen effluvium.

66. The method of claim 61, wherein said mammal is undergoing therapy with methotrexate, nonsteroidal anti-inflammatory drugs, or beta blockers.

67. A method of blocking the permeability transition pore in mitochondria, comprising contacting said mitochondria with a compound of Formula I of claim 1, or with a pharmaceutically acceptable derivative thereof.

68. A method of inhibiting breakdown of mitochondrial metabolism in cells which undergo oxidative stress, comprising contacting said cells with a compound of Formula I of claim 1, or with a pharmaceutically acceptable derivative thereof.

69. A method of preventing or delaying cell death in a cell subjected to calcium overload, comprising contacting said cell with a compound of Formula I of claim 1, or with a pharmaceutically acceptable derivative thereof

70. A method of preventing, mitigating, or delaying excitotoxic or hypoglycemic injury to cells, tissues, or organs, comprising contacting said cells, tissues, or organs with a compound of Formula I of claim 1, or with a pharmaceutically acceptable derivative thereof.

71. A method of inhibiting breakdown of energy metabolism and cell death of mammalian cells following physiological induction of programmed cell death, comprising contacting said cells with a compound of Formula I of claim 1, or with a pharmaceutically acceptable derivative thereof.

72. A method of preventing or delaying death of cultured cells in large scale or commercial scale cell culture, comprising contacting said cells with a compound of Formula I of claim 1, or with a pharmaceutically acceptable derivative thereof.

73. A method of treating or preventing ischemic injury or ischemia/reperfusion injury in a mammal, comprising administering to said mammal an effective amount of a compound of Formula I of claim 1, or of a pharmaceutically acceptable derivative thereof.

74. The method of claim 73, wherein said ischemic injury or ischemia/reperfusion injury is mesenteric infarction, bowel ischemia, hepatic infarction, renal infarction, splenic infarction, or ischemic heart disease.

75. The method of claim 74, wherein said ischemic heart disease is congestive heart failure, myocardial ischemia, or coronary heart disease.

76. A method of treating an ophthalmic disorder in a mammal, comprising administering to said mammal a therapeutically effective amount of a compound of Formula I of claim 1, or of a pharmaceutically acceptable derivative thereof.

77. The method of claim 76, wherein said ophthalmic disorder is glaucoma, ischemic retinopathy, vascular retinopathy, or degeneration of the photoreceptor cell layer.

78. A method of treating Reye's syndrome in a patient, comprising administering to said patient a therapeutically effective amount of a compound of Formula I of claim 1, or of a pharmaceutically acceptable derivative thereof.

79. A method of preventing or reducing tissue damage of organs used in organ transplantation surgery, comprising contacting said organs with a compound of Formula I of claim 1, or with a pharmaceutically acceptable derivative thereof.

80. A method of treating an infection or infestation with pathogenic protozoan or helmintic parasites, comprising contacting said parasites with a compound of Formula I of claim 1.

81. A method of treating an infection with pathogenic protozoan or helmintic parasites in an animal, comprising administering to said animal a therapeutically effective amount of a compound of Formula I of claim 1, or with a pharmaceutically acceptable derivative thereof.

82. The method of claim 81, wherein said infection is malaria, river blindness, lymphatic filariasis, intestinal roundworm infection, tapeworm infection, pinworm infection, toxoplasmosis, leishmaniasis, trypanosomiasis, or bilharzia.

83. A method for treating a virus infection in a mammal, comprising administering to said mammal a therapeutically effective amount of a compound of Formula I of claim 1, or of a pharmaceutically acceptable derivative thereof.

84. The method of claim 83, wherein said virus is a human immunodeficiency virus.
